(19) United States
(12) Patent Application Publication
(10) Pub. No.: US 2010/0004234 A1
(43) Pub. Date: Jan. 7, 2010

(54) SPECIFIC KINASE INHIBITORS

(75) Inventors: Daniel V. Santi, San Francisco, CA (US); Ralph C. Reid, San Rafael, CA (US); C. Richard Hutchinson, Cross Plains, WI (US); Kurt F. Sundermann, Burlingame, CA (US); Janice Lau, San Mateo, CA (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Kosan Biosciences Incorporated

(21) Appl. No.: 12/536,884

(22) Filed: Aug. 6, 2009

Related U.S. Application Data
(63) Continuation of application No. 11/236,244, filed on Sep. 26, 2005.
(60) Provisional application No. 60/613,680, filed on Sep. 27, 2004.
(60) Provisional application No. 60/629,575, filed on Nov. 18, 2004.
(60) Provisional application No. 60/698,520, filed on Jul. 11, 2005.

(51) Int. Cl. A61K 31/365; C12N 5/00; A61K 31/5377; A61K 31/496; A61K 31/4523; C07D 493/04; A61P 35/00

(52) U.S. Cl. 514/232.8; 435/375; 514/254.11; 514/321; 514/450; 544/147; 544/378; 546/197; 549/268

(57) ABSTRACT

Resorcylic acid lactones having a C5-C6 cis double bond and a ketone at C7 and other compounds capable of Michael adduct formation are potent and stable inhibitors of a subset of protein kinases having a specific cysteine residue in the ATP binding site.

### CROSS-REFERENCE TO RELATED APPLICATIONS

This application claims the benefit under 35 U.S.C. §119(e) of U.S. Provisional Applications Nos. 60/613,680, filed Sep. 27, 2004; 60/629,575, filed Nov. 18, 2004; and 60/698,520, filed Jul. 11, 2005; the disclosures of which are incorporated herein by reference. This application is a continuation of U.S. application Ser. No. 11/236,244, filed Sep. 26, 2005.

### BACKGROUND OF THE INVENTION

1. Field of the Invention

The present invention provides compounds that inhibit specific protein kinases and are useful in the treatment of human disease. The invention relates to the fields of chemistry, biochemistry, molecular biology, medicine, and pharmacology.

2. Description of Related Art

Molecularly targeted cancer drugs offer significant promise in the current and future treatment of cancer. Numerous proteins have been identified as playing critical roles in specific steps in cell signaling. These signaling pathway proteins are attractive targets for cancer drugs as they permit a degree of selectivity over normal healthy cells (Sausville et al., Annu Rev Pharmacol Toxicol (2003) 43:199-231). Because cell signaling typically involves multiple pathways, however, specific inhibition of a particular signaling pathway protein may be insufficient to obtain a desired therapeutic result. Conversely, non-specific inhibition of multiple signaling pathways may have a detrimental result on normal cells, thus defeating the purpose of targeting the signal pathway protein in the first instance.

Successful drug development in this area is accordingly difficult and unpredictable. A compound developed based on its ability to inhibit a particular cell signaling pathway may work for a particular indication only if it inhibits another cell signaling pathway protein as well, a property that current technology does not allow one to predict. For example, Gleevec (imatinib mesylate, STI-571, Novartis) was designed as a specific inhibitor of the Bcr-Abl tyrosine kinase, but its efficacy depends on its ability to inhibit c-Kit and other tyrosine kinases as well. Thus, Gleevec does indeed inhibit the Bcr-Abl tyrosine kinase important in maintaining chronic myelogenous leukemia (CML) cell function (Hernandez-Boluda et al., Drugs Today (Barc) (2002) 38:601-13) and so is effective against CML, but its efficacy also depends in part on its ability to inhibit the c-Kit tyrosine kinase, which also makes it effective against gastrointestinal stromal tumors in which the c-Kit tyrosine kinase is elevated by mutation (Blanke et al., Curr Treat Options Oncol (2001) 2:485-91).

Gleevec also illustrates the value of targeting protein kinases in cancer drug development. Members of the large family of over 500 protein kinases are involved in most, if not all, important cell signaling pathways. Four major signaling pathways or cascades, one responsive to extra-cellular mitogens and others to stress signals, each controlled by a protein kinase and each containing multiple other protein kinases, play vital roles in cancer cell division and cellular stress responses and so are of intense interest for the development of anti-cancer and anti-inflammatory drugs. However, the unpredictable nature of how a compound will affect the many different protein kinases in the multiple different signaling pathways continues to slow drug development.

The interception of cell signaling pathways involving aberrant mitogen activated protein kinases, the so-called MAP (mitogen activated protein) kinases or MAPK enzymes (Chen et al., Chem Rev (2001) 101:2449-76; Pearson et al., Endocr Rev (2001) 22:153-83), has emerged as an important direction for the discovery and development of new types of cancer drugs (English et al., Trends Pharmacol Sci (2002) 23:40-45; Kohno et al., Prog Cell Cycle Res (2003) 5:219-24; Sebolt-Leopold, Oncogene (2000) 19:6594-99). One of the MAPK-dependent pathways enables the transmission of signals from extracellular signals, such as epidermal growth factor (EGF) and vascular endothelial derived growth factor (VEGF), which bind to a corresponding receptor in the cell membrane, EGFR [HER] and VEGFR, respectively, which sends the signal on to the cell nucleus via intermediary kinases and kinase targets (e.g., the ERK pathway: Ras, Raf-1, A-Raf, B-Raf (BRAF), MEK1 and MEK2, which are collectively referred to herein as MEK1/2, and ERK1 and ERK2, which are collectively referred to herein as ERK1/2). The latter proteins ultimately govern expression of genes that control vital cell functions such as proliferation, growth, motility and survival. Two to three other protein kinase pathways respond to “stress signals”.

Small-molecule, non-protein drugs targeted at specific protein kinases are in development (English et al., Trends Pharmacol Sci (2002) 23:40-45; Kohno et al., Prog Cell Cycle Res (2003) 5:219-24; Sebolt-Leopold, Oncogene (2000) 19:6594-99; Noble et al., Science (2004) 303:1800-05), and three have been approved for use: Gleevec; gefitinib (Iressa; Barker et al., Bioorg Med Chem Lett (2001) 11:1911-14); and erlotinib (Tarceva). The dearth of approved small molecule kinase inhibitors as drugs illustrates the unpredictability of current methods. While compounds that inhibit a particular protein kinase can be designed and evaluated with the aid of 3D structures of their targets (Noble et al., Science (2004) 303:1800-05), clinical experience has shown that many compounds fail to meet the optimistic expectations based on preclinical activity (Sausville et al., Annu Rev Pharmacol Toxicol (2003) 43:199-231; Dancey et al., Nat Rev Drug Discov (2003) 2:296-313). This failure results in part from the difficulty of predicting an inhibitor's effects on the myriad other protein kinases in important cell signaling pathways based simply on its ability to inhibit a particular kinase. Hence, there is considerable need for new and improved drugs that target specific protein kinases and specific subsets of protein kinases, and methods for identifying and using known kinase inhibitors in the treatment of cancer and other diseases.

Such drugs could have significant impact on the treatment of human disease. For example, in cancer therapy, pharmacological inhibitors of the MAPK pathways could target any of several different proteins in the signaling process (English et al., Trends Pharmacol Sci (2002) 23:40-45; Kohno et al., Prog Cell Cycle Res (2003) 5:219-24). Proteins of particular interest for cancer therapy include the MAPK/extracellular signal-related kinase (ERIC) kinases, called MEKs or MKKs, especially those that act on the ERK branch of MAPK signaling, which involves Ras/Raf-1, A-Raf and/or B-Raf, MEK1/2, and ERK1/2 (see FIG. 1). The G-protein Ras relays signals from the mitogen-activated growth factor receptors to Raf-1, A-Raf and/or B-Raf that phosphorylate and thus activate the dual-specific serine/threonine and tyrosine kinases MEK1/2, which then activate ERK1/2. The Ras/Raf/MEK/ERK pathway is reportedly one of the best-characterized signaling pathways involved in the development and propagation of human cancers and has been proposed as a target for anti-cancer drug development (Kohno et al., Prog Cell Cycle Res (2003) 5:219-24; Dancey et al., Nat Rev Drug Discov (2003) 2:296-313).

However, the complex set of pathways that control cell division and movement in cancer, inflammation, and normal cell vital functions suggests that compounds that inhibit only a single pathway or branch of a complex of pathways may not be efficacious. Compounds that correctly inhibit multiple pathways, without deleterious non-specific activity harmful to normal cells, are difficult to design and test. Compounds targeting the MEK1/2 kinases illustrate the problem.

MEK1/2 kinases have two attractive features as targets for the development of antitumor (anticancer) drugs: (1) they are at a crucial point of pathway convergence that integrates input from a variety of mitogen-activated protein kinases through Ras; and (2) they have restricted substrate specificity, with the MAPKs ERK1/2 the only known substrates of importance. Constitutive activation or enhanced activity of MEK1/2 has been detected in a number of primary human tumor cells (Hoshino et al., Oncogene (1999) 18:813-22); indeed, a single mutation in B-Raf can constitutively activate the ERK pathway, and the mutant gene is oncogenic. The major B-Raf mutation is V599E (the correct name of this mutation is V600E although most literature, particularly older literature, refers to it as V599E) (Davies et al. Nature (2002) 417:949-54). However, only a few small-molecule or antisense inhibitors of MEK1/2 [PD184352/CI-1040 (Pfizer), U-0126 (Promega) and a compound from Wyeth-Ayerst (Zhang et al., Bioorg Med Chem Lett (2000) 10:2825-28)] or Raf-1/B-Raf [BAY-439006] (Lyons et al., Endocr Relat Cancer (2001) 8:219-25) have been reported to be in preclinical development or clinical trials (Kohno et al., Prog Cell Cycle Res (2003) 5:219-24; Dancey et al., Nat Rev Drug Discov (2003) 2:296-313). So far, no specific and potent ERK1/2 inhibitors have been reported.

Examination of the properties of some of the known MEK1 inhibitor compounds reveals that their efficacy may depend in part on their ability to inhibit multiple pathways. PD184352 and U-0126 inhibit MEK1 and are non-competitive with ATP, most likely functioning as allosteric inhibitors that bind outside the ATP binding sites. These compounds also inhibit activation of the MEK5-ERK5 pathway at similar concentrations. Both compounds have anti-tumor activity in animals, especially against tumors in which the ERK pathway is constitutively activated, and are reportedly in clinical trials (Dancey et al., Nat Rev Drug Discov (2003) 2:296-313).

However, even if these MEK1 inhibitor compounds in development can target multiple signaling pathways, their success as drugs is by no means certain. If inhibition of multiple signaling pathways is required, the drugs must inhibit at least one protein kinase in each pathway with sufficient potency to bring about the desired therapeutic result. Moreover, such drugs are often primarily cytostatic agents and may not kill the tumor cell efficiently, making resistance and recurrence more likely. For drugs that are rapidly reversible inhibitors, their removal, or a decline in their cellular level, permits the re-initiation of tumor cell proliferation. Inhibitors that bind covalently can be more effective than the reversible protein kinase inhibitors (Noble et al., Science (2004) 303:1800-05), as has been shown for drugs that inhibit EGFR and Her-2, in which the compounds form a covalent bond by Michael addition to a cysteine residue in the ATP pocket (Wissner et al., Bioorg Med Chem Lett (2002) 12:2893-97; Baslega et al., Oncology (2002) 63 Suppl 1:6-16; Wissner et al., J Med Chem (2003) 46:49-63). There remains a need for protein kinase inhibitors that can be developed as drugs, and inhibitors that covalently modify their targets to inhibit them could be particularly useful in the treatment of human disease.

In the search for protein kinase inhibitors, natural products have been studied, because such compounds have proven invaluable as leads for drugs that affect signaling pathways (Newman et al., Curr Cancer Drug Targets (2002) 2:279-308). The class of fungal natural products known as the “resorcylic acid lactones,” also referred to herein as “RALs” (see FIG. 2), includes the zearalenones, which are estrogenic and have been used as anabolic agents in animals (e.g., zearalanol), as well as (5Z)-7-oxozeaneol, hypothemycin, Ro-09-2210, and L-783,277, which have been reported to inhibit cell proliferation (Zhao et al., J Antibiot (Tokyo) (1999) 52:1086-94; Camacho et al., Immunopharmacology (1999) 44:255-65) and to have antitumor properties (Zhao et al., J Antibiot (Tokyo) (1999) 52:1086-94; Tanaka et al., Jpn J Cancer Res (1999) 90:1139-45). Also of interest is their ability to inhibit JNK/p38 signaling in cells (Takehana et al., Biochem Biophys Res Commun (1999) 257:19-23), the autophosphorylation of the platelet-derived growth factor (PDGF) receptor (Giese et al., U.S. Pat. No. 5,728,726 (1998), MEK1/2 (Zhao et al., J Antibiot (Tokyo) (1999) 52:1086-94; Dombrowski et al., J Antibiot (Tokyo) (1999) 52:1077-85; Williams et al., Biochemistry (1998) 37:9579-85) or TAK1 (a MEKK) (Ninomiya-Tsuji et al., J Biol Chem (2003) 278:18485-90) in vitro with low nanomolar IC₅₀ values. Despite their interesting activities, however, no resorcylic acid lactone has been tested in humans, or approved as a drug.

The resorcylic acid lactone L-783,277 inhibits the phosphorylation of purified MEK1 (IC₅₀ 4 nM) but not PKA, PKC or Raf. The inhibition is competitive with ATP and a 60 min. pre-incubation reduced the IC₅₀ value for MEK1 10-fold (Zhao et al. J. Antibiot (Tokyo) (1999) 52:1086-94). Pre-incubation of MEK1 with L-783,277 for 30 minutes, followed by gel filtration, led to the recovery of inactive MEK1 protein indicating that L-783,277 tightly binds to MEK1. However, the 5E C═C isomer was ˜100-fold less potent, and the 7-dihydro hydroxyl isomers were 400 to 5000-fold less potent than L-783,277, but no clear SAR emerged (Zhao et al., supra). Hypothemycin (see FIG. 2), which is structurally similar to L-783,277 but has an 11,12-epoxide moiety, is 4-fold less potent as a MEK1 inhibitor (Zhao et al., supra). Ro-09-2210 is a potent inhibitor of MEK1 (IC₅₀ 59 nM) and is claimed in unpublished work (see Williams et al., Biochemistry (1998) 37:9579-85) to inhibit MEK4, 6, and 7 with 4 to 10-fold higher IC₅₀ values. The (5Z)-7-oxozeaneol has similar potency against the TAK1 MEKK enzyme (IC₅₀ 8 nM) and exhibited a lesser inhibition of rat MEK1 (IC₅₀ 411 nM) (Ninomiya-Tsuji et al., J Biol. Chem. (2003) 278:18485-90).

The reason for potent inhibition of these target kinases by such analogs was, prior to the present invention, unknown, and, no comprehensive evaluation against the more than about 500 protein kinases encoded, in the human genome (the “kinome”) has been performed for these or any other compounds. Such evaluation is currently not possible, because protein kinase assays have been developed for only about ˜150 of these kinases. There remains a need for methods for assessing whether a compound can inhibit a kinase and for determining which kinases a compound will inhibit. Without such methods and in the absence of an assessment of multiple kinases in vitro, which has not been reported for any of the RAL compounds, one cannot determine a compound's relative selectivity among protein kinase family members and so cannot readily evaluate a compound's utility in the treatment of human disease.

Thus, there remains a need for methods of identifying protein kinase inhibitors and for assessing their relative selectivity in the kinome and especially for the various protein kinases involved in disease. With such methods, one could identify and select compounds that productively inhibit protein kinases from multiple cell signaling pathways that are directly related to the biology of a given disease. One could select inhibitors that inhibit only specific targets and signal transduction pathways, formulate them as drug products and administer them to treat diseases in which inhibition of those targets provides a therapeutic effect, including against diseases such as cancer, inflammation, and other conditions. The present invention meets these needs and provides methods, compounds, and pharmaceutical products, as described below.

### BRIEF SUMMARY OF THE INVENTION

In a first aspect, the present invention provides methods for inhibiting a protein kinase using a distinct subclass of protein kinases with a compound capable of Michael adduct formation with the protein kinase. The subclass of kinases is composed of kinases that have a cysteine residue (Cys) located between two, and immediately adjacent to one, of the highly conserved aspartate residues (Asp) in the protein kinase that interact with the phosphate target and the Mg²⁺ complexed with the phosphates of the ATP. These amino acids in the protein kinase are located in the region known as the ATP-binding site. In the methods of the invention, a protein kinase having such a Cys residue is inhibited by contact with a compound that can form a Michael adduct at the Cys residue. The Michael adduct formation results in the formation of a covalent bond between the inhibitor and the kinase, thus making the inhibition essentially irreversible.

In one embodiment, a mixture of protein kinases, including one or more from the subclass containing the Cys and one or more from kinases that lack the critical Cys residue, is contacted with a compound comprising a moiety capable of forming a reversible complex with enzymes containing the Cys residue, and then forming a Michael adduct with this Cys residue. In one embodiment, this moiety is Z-enone (Z-alpha, beta-unsaturated carbonyl moiety). In one embodiment, this moiety is contained in a resorcylic acid lactone or derivative that contains a cis carbon-carbon double bond at positions 5-6 conjugated to a carbonyl at position 7 (an alpha, beta-unsaturated ketone; see FIG. 2) or a bioisostere of such a moiety, such as an ester, amide, bis-lactone, sulfonamide, or sulfone. In the method, only one or more protein kinases from the subclass of kinases containing the critical Cys residues is inhibited by Michael adduct formation; protein kinases lacking the Cys residue are either not inhibited (or not to the same degree) or are inhibited by a different mechanism not involving Michael adduct formation.

The methods of the invention can be practiced with a variety of mixtures. In one embodiment, the mixture is a reaction mixture employed in an in vitro assay. In another embodiment, the mixture is a cell or cell fraction. In another embodiment, the mixture contains cells and media, as obtained from a cell culture assay. In another embodiment, the mixture is a bodily fluid or tissue. In one important embodiment, the mixture includes diseased tissues in a human or other mammal undergoing medical treatment.

The protein kinase inhibitors useful in the methods typically inhibit at least two or more different protein kinases in achieving their therapeutic effect. The compounds useful in the methods of the invention can, for example, inhibit two or more different protein kinases, one from each of at least two different signaling pathways, or inhibit two or more different protein kinases in the same pathway, or both, in achieving their desired effect. In some embodiments, the compounds used in the methods of the invention inhibit at least three different protein kinases in achieving their intended effect.

In one embodiment, a compound of the invention is administered to inhibit multiple enzymes in the ERK pathway to achieve a desired therapeutic effect. In one embodiment, these enzymes are MEK1/2 and ERK1/2. In one embodiment, a compound of the invention inhibits multiple enzymes in the ERK pathway as well as a mitogen receptor kinase. In one embodiment, a compound inhibits the VEGF receptor and, through inhibition of the ERK pathway, VEGF production. Such compounds of the present invention are particularly useful in the treatment of diseases involving angiogenesis, including but not limited to cancer and macular degeneration, because they not only inhibit the production of VEGF via inhibition of the pathway that leads to its production but also inhibit its receptor VEGFR directly.

In one embodiment, the protein inhibited by a compound of the invention is a MAP kinase. In one embodiment, the different signaling pathways inhibited include at least one mitogen-induced pathway and one stress-induced pathway. In one embodiment, at least one of the protein kinases is a MEK. In one embodiment, at least one of the protein kinases is a member of the MAPKK family. In one embodiment, at least one of the protein kinases is a tyrosine receptor kinase, including but not limited to wild-type and mutant PDGFRA, PDGFRB, FLT-3, c-KIT, and the VEGF receptors. In one embodiment, at least one of the protein kinases is a VEGF receptor, including VEGFR1, VEGFR2 (also known as KDR), and VEGFR3. In one embodiment, at least one of the protein kinases is FLT3. In one embodiment, at least one of the protein kinases is c-KIT. In one embodiment, at least one of the protein kinases is PDGFRA or PDGFRB.

In one embodiment, the protein kinase inhibited by a compound useful in the methods of the invention is selected from the group consisting of AAK1, APEG1 splice variant with kinase domain (SPEG), BMP2K (BIKE), CDKL1, CDKL2, CDKL3, CDKL4, CDKL5 (STK9), ERK1 (MAPK3), ERK2 (MAPK1), FLT3, GAK, GSK3A, GSK3B, KIT (cKIT), MAP3K14 (NIK), MAP3K7 (TAK1), MAPK15 (ERK8), MAPKAPK5 (PRAK), MEK1 (MKK1, MAP2K1), MEK2 (MKK2, MAP2K2), MEK3 (MKK3, MAP2K3), MEK4 (MKK4, MAP2K4), MEK5 (MKK5, MAP2K5), MEK6 (MKK6, MAP2K6), MEK7 (MKK7, MAP2K7), MKNK1 (MNK1), MKNK2 (MNK2, GPRK7), NLK, PDGFR alpha, PDGFR beta, PRKD1 (PRKCM), PRKD2, PRKD3 (PRKCN), PRPF4B (PRP4K), RPS6KA1 (RSK1, MAPKAPK1A), RPS6KA2 (RSK3, MAPKAP1B), RPS6KA3 (RSK2, MAPKAP1C), RPS6KA6 (RSK4), STK36 (FUSED_STK), STYK1, TGFBR2, TOPK, VEGFR1 (FLT1), VEGFR2 (KDR), VEGFR3 (FLT4) and ZAK.

In one embodiment, the compound used in a method of the invention inhibits at least two of the foregoing proteins. In another embodiment, at least 3 of the protein kinases are inhibited.

In a second aspect, the present invention provides methods for treating disease that comprise administering a compound capable of forming a Michael adduct with a protein kinase containing the target Cys residue to a subject in need of treatment. In one embodiment, the subject is a mammal. In one embodiment, the subject is a human. In one embodiment, the compound is a resorcylic acid lactone or derivative compound. Prior to the present invention, it was impossible to a priori predict the specificity of any resorcylic acid lactone or any kinase inhibitor for each different kinase in the kinome. Knowledge of kinase targets required experimental testing, and in vitro assays have to date been developed for only ˜150 of the more than 500 kinases in the kinome. Because of the large number of protein kinases and their fundamental role in a variety of normal and disease processes, one could not determine whether such compounds or other compounds, even if demonstrated to inhibit a particular kinase, would have the specificity required to inhibit a kinase and treat disease or instead would be so non-specific that vital normal processes would be harmed. In contrast, because the kinase targets in the present invention are identified by their molecular structure as either capable or not of forming the Michael adduct, the entire repertoire of targets can be identified from available sequence data of the kinome.

The present invention also provides pharmaceutical compositions and methods for administering them for the treatment of disease. In one embodiment, the methods include co-administration of another drug with the protein kinase inhibitor. In one embodiment, the other drug is an anti-cancer drug. In another, the drug is an anti-inflammatory drug. In another embodiment, the drug is another protein kinase inhibitor. In one embodiment, the pharmaceutical composition comprises a compound, including but not limited to a resorcylic acid lactone or derivative, that has specificity for and can form a Michael adduct with one or more proteins of the subclass of protein kinases containing the critical Cys residue and targets a disease or condition. In one embodiment, the pharmaceutical composition is administered to achieve therapeutic effect without unwanted side effects that would otherwise arise from inhibition of a protein kinase that does not contain the target Cys residue (located between the two and adjacent to one of the conserved Asp residues in the ATP binding site of the protein kinase).

### BRIEF DESCRIPTION OF THE DRAWING(S)

FIG. 1 shows a schematic representation of the ERK/MAPK signaling pathway.

FIG. 2 shows the chemical structures of certain resorcylic acid lactones.

FIG. 3 and FIG. 4 show an X-ray structure of the kinase ERK2 having hypothemycin covalently bound thereto.

FIG. 5 shows, in bar graph form, log GI₅₀ values (the amount of drug required to achieve 50% growth reduction) for hypothemycin against the 60 cell line NCI panel. Cell lines most sensitive to the compound are depicted with bars pointing to the right from the vertical mean activity.

FIG. 6 shows comparative xenograft data for hypothemycin and a non-RAL drug.

FIG. 7 compares the mass spectra of tryptic digests of the kinase ERK2 in the presence and absence of hypothemycin.

FIG. 8 shows the effect of hypothemycin on the phosphorylation of the kinase ERK in Colo829 cells having a BRAFV599E mutation.

FIG. 9 shows the duration of the inhibition of the phosphorylation of the kinase ERK by hypothemycin in HT29 cells having a B-Raf V599E mutation.

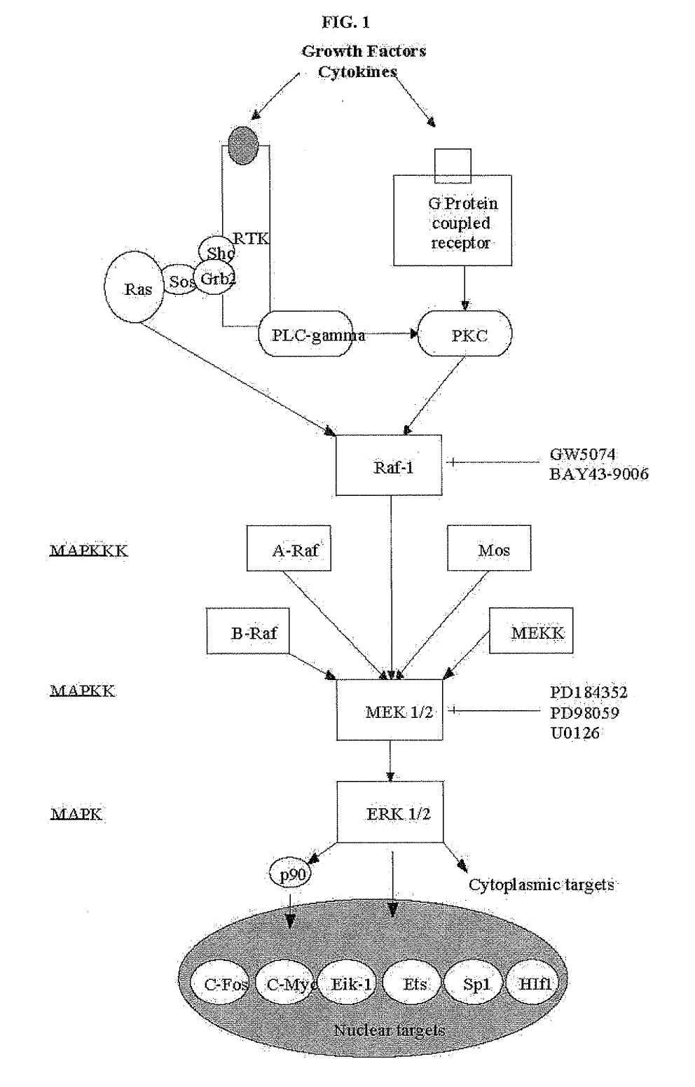

### DETAILED DESCRIPTION OF THE INVENTION

The human genome is currently reported to have 510 identifiable genes of standard eukaryotic protein kinase type—referred to as the human “kinome” (Kositch et al., Genome Biology 2002, 3 (9): RESEARCH 0043). The protein kinase family offers a rich source of targets for therapeutic intervention, because its members play key roles in many disease processes, including inflammation and cancer. However, the large number of proteins in this family and the many different cell signaling pathways containing them makes finding a drug both sufficiently active and specific to be of medical use difficult and unpredictable. The present invention provides compounds, compositions, and methods for inhibiting an identifiable specific subset of protein kinases from multiple different cell signaling pathways in multiple organisms and so represents a significant advance in the effort to target protein kinases in the treatment of disease.

In the protein kinase family, two highly conserved Asp residues [D167 and D185, using residue numbers from PKA-Calpha (NP_{—}002721)] have been assigned the following roles: the first accepts the H⁺ from the phosphate OH of ATP; the second interacts with the Mg²⁺ that is complexed with phosphates of ATP (thus contributing to the positioning of the gamma-phosphate for transfer). In this region, immediately preceding the second Asp (corresponding to position 184 of PKA-Calpha) is a variable position that is Cys in about 10% of human kinases (˜50/510). Its position is necessarily in the ATP binding site region, due to its proximity to the second Asp.

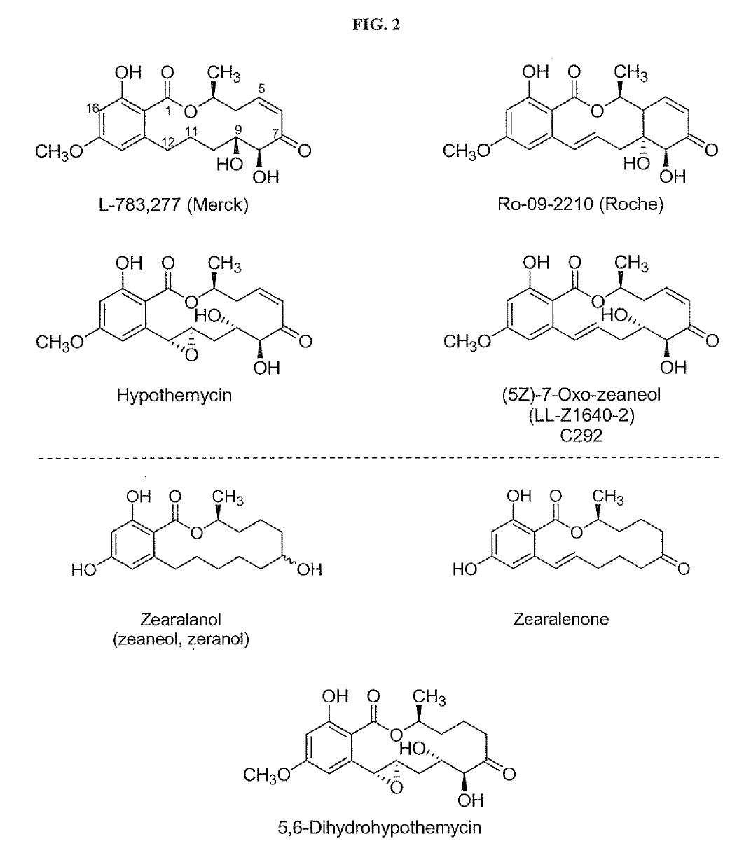

The present invention arose in part from the discovery that certain resorcylic acid lactones that inhibit these Cys-containing kinases share a common structural feature. These compounds have in common a cis double bond conjugated to a carbonyl at positions 5-7 (see, e.g., the first four structures in FIG. 2). Such compounds have the following molecular scaffold (with the numbering used in this specification also shown):

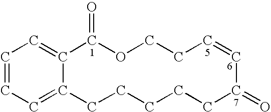

After formation of an initial reversible enzyme-inhibitor complex, proximity of this structure within the complex to a Cys side chain in the kinase domain/ATP-binding site can lead to the subsequent formation of a very slowly reversible or effectively irreversible Michael adduct and provide a mechanism for extremely potent inhibition.

A Michael adduct is formally the product of the 1,4-addition of a nucleophilic species to a conjugated electrophilic double bond, as illustrated by the equation below:

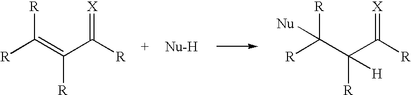

wherein X is typically O or NR and Nu is typically a carbon, nitrogen, oxygen or sulfur based nucleophilic group. The conjugated electrophilic double bond is typically in an α,β-unsaturated ketone, aldehyde, or ester moiety, but may also be in an unsaturated nitrile, sulfone, or nitro moiety. For the purposes of this application, the term “Michael adduct” refers to the formal product of such 1,4-addition without regard to the exact mechanism of formation of the product and further encompasses tautomeric forms of such formal products, including for example enolized forms.

Examination of the available published data in view of the present invention reveals that the Cys is present in the few MAP kinases reported to be sensitive to resorcylic acid lactones having such a structure, and absent in those reported to be insensitive. It has been reported, for example, that a cis-enone resorcylic acid lactone inhibits MEK1, 4, 6 and 7, as well as the MAPKKK TAK1 and mitogen receptor tyrosine kinase PDGFR. About 10 kinases that do not have the target Cys residue have been reported not to be inhibited by certain cis-enone resorcylic acid lactones.

Thus, the present invention provides resorcylic acid lactones and analogs containing this structure and methods for their use in selectively inhibiting the up to 50 kinases containing cysteine residues in or proximal to the kinase domain ATP-binding site. The present invention also provides pharmaceutical compositions containing such compounds and methods for treating disease with them. In particular, the specificity of the compounds of the invention can be predicted for the multiple kinase targets relevant to a particular disease state; the methods of the invention provide for the treatment of diseases in which the targets inhibited play a causative or contributive role.

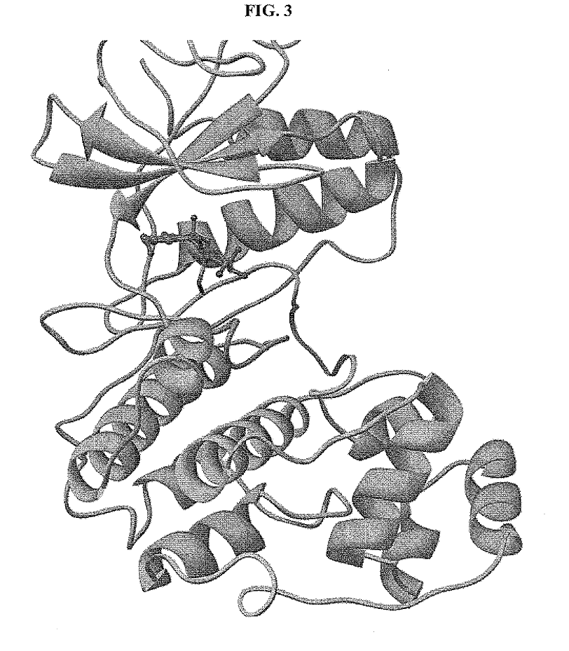

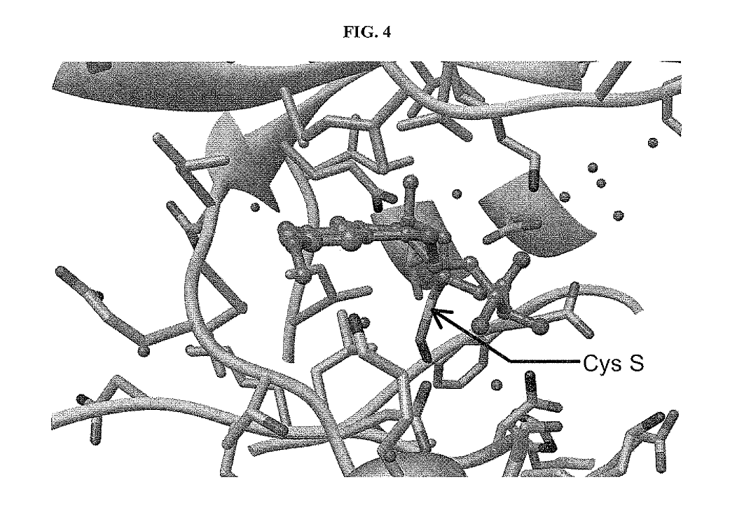

The correctness of this model is evidenced by the X-ray structure of a covalent complex of the kinase EKR2 and hypothemycin. In a 2.5 angstrom resolution structure, FIG. 3 shows the complex with the ERK2 N-terminal lobe on top, the C-terminal lobe at the bottom, and hypothemycin covalently bound to the hinge region. FIG. 4 shows a close-up view of the hinge region, pointing out the cysteine sulfur that has added, in a Michael reaction, across the enone double bond of hypothemycin.

Potent inhibition of protein kinases by the resorcylic acid lactone inhibitors described herein requires that the inhibitors pass two “selectivity filters” imposed by the target enzymes. First, they must reversibly bind to the enzyme with a reasonably tight association constant. This reversible-binding filter depends on the complementary topology of the inhibitor and enzyme, as well as formation of reversible energy-forming bonds (e.g. hydrogen bonds, ionic interactions, hydrophobic interactions). The second filter involves the formation of a covalent bond between the target thiol of the enzyme and the beta-carbon of the enone moiety of the inhibitor. This filter requires the presence of an appropriate Cys residue within the enzyme-inhibitor complex, and its efficacy depends on the appropriate juxtapositioning of the reactive thiol with the Michael-acceptor carbon atom. Some resorcylic acid lactones may not pass the first filter of a kinase (reversible binding), and hence never encounter the second (covalent binding); some resorcylic acid lactones will pass the first filter of a kinase, but the kinase will not have a Cys residue to form a covalent bond. Indeed, examples of both are cited herein. The targets of interest to the resorcylic acid lactones in the present invention are those that pass both filters.

Most kinase inhibitors have been discovered by routine screening followed by optimization against one or several kinases. As a result, they are developed to pass the first filter (described above), and their specificity depends upon how many different kinases share similar topology and reversible interactions at their binding sites. Because the ATP site of protein kinases are highly conserved, reversible inhibitors that bind to this site are likely to inhibit many kinases, but in an unpredictable and apparently indiscriminate fashion. For example, in a panel of some 120 kinases, the compound identified as Sugen 11248 inhibits some 79 kinases with a range of Kᵢ values of 0.002 to 6.6 μM (Fabian et al., Nat. Biotechnol. 2005; 23 (3):329-36); of these, some 56 kinases show Kᵢ values of <0.1 μM and therefore may be relevant in vivo targets. With covalent binding to resorcylic acid lactones as a second filter, the discrimination among kinases is uniquely and greatly enhanced, because only the subset containing the target Cys residue is inhibited irreversibly.

The covalent nature and, in-effect, irreversibility of the kinase-resorcylic acid lactone interaction provides additional benefits relevant to drug action and the methods of administration provided by the present invention. For example, because resorcylic acid lactones have different reversible affinities (Kᵢ) and rates of covalent inactivation (k_{inact}) with different kinases, by controlling the exposure (concentration×time) of a mixture of kinases to resorcylic acid lactones, selective inhibition of certain kinases may be achieved. This is reflected by the “specificity constant” of a given resorcylic acid lactone for a given kinase, which is, in effect, the second order rate constant for covalent attachment at very dilute inhibitor and kinase concentrations. For example, hypothemycin has a Kᵢ for ERK2 of 2 μM with a t_{1/2} of 3 min for inactivation (k_{inact}/Kᵢ=1.9E+03); for KDR, the Kᵢ is 0.01 μM with a t_{1/2} of about one min for inactivation (k_{inact}/Kᵢ=5E+05). It can be calculated, that by treating the two kinases with 0.1 μM (Kᵢ ERK>0.1 um>Ki KDR) for ˜10 minutes, > 98% of KDR activity can be inhibited under conditions where < 5% of ERK activity is inhibited. Further, if the exposure is sufficient to allow covalent inhibition to go to completion, administration of the drug can be withdrawn to relieve any reversible inhibition of non-Cys kinases, but maintain inhibition of the specific set of kinases that have been covalently modified.

The invention can be appreciated in part by comparing hypothemycin, which contains the Michael adduct-forming structure, and zearalenone and 5,6-dihydrohypothemycin, which do not (see FIG. 2), and their respective abilities to inhibit the activation of ERK1/2. Hypothemycin has been reported to inhibit the activation of ERK1/2 in human T cells, but zearalenone not, when the compounds are tested at 0.3 to 3 μM (see Camacho et al., 1999, Immunepharmacology 44 (3):255-265). An examination of the corresponding human protein kinase amino acid sequences shows appropriately positioned Cys residues in ERK1/2.

Examination of homology models for any of a variety of protein kinases, such as MEK1/2 or ERK1/2, illustrates that the positioning of a resorcylic acid lactone in the ATP-binding site region of the protein kinase would allow for Michael adduct formation. For example, a homology model of the MEK1 ATP-binding site supports a mechanism in which the alpha, beta-unsaturated carbonyl-containing resorcylic acid lactone or derivative can inhibit protein kinases containing the critical Cys residue by Michael adduct formation.

Such models allow, in view of the present invention, one not only to predict the structures of novel kinase inhibitors that can inhibit a protein kinase susceptible to inhibition by Michael adduct formation but also to identify known compounds having such structures that are useful in the methods of the invention. In one embodiment, the compounds useful in the methods of the invention are known, previously tested compounds, which are employed in a method of the invention in which the mixture employed includes kinases against which the specificity of inhibition of the known compound has not been tested or determined. In another embodiment, the compounds of the invention are novel compounds that have not previously been made or tested.

To appreciate the advances provided by the present invention, one must appreciate that it is well established that essentially all protein kinase inhibitors inhibit multiple kinases, and that the response of a cell to a particular inhibitor involves simultaneous inhibition of two or usually more kinases. It follows that the specificity and efficacy of any given inhibitor will depend on its kinase inhibition profile, and that different profiles have different effects on a cell. The kinase profile of most known kinase inhibitors can only be determined experimentally and is therefore limited by the number of enzymes available for assay. For example, profiles of the inhibitory activity of a number of kinase inhibitors against a large panel of 120 kinases have been reported (Fabian et al., Nat. Biotechnol. 2005; 23 (3):329-36). Imatanib (Gleevec) inhibited ten out of 120 kinases, and BAY 43-9006 inhibited 19 out of 120 kinases with Kᵢ<0.1 μM, but it is not known how many or which of the remaining 300 kinases currently unavailable for screening are inhibited by these compounds. In contrast, the present invention provides the definitive list of targets in the entire human kinome inhibited by the resorcylic acid lactones (RALs) of the invention, capable of forming Michael adducts with those targets at the critical Cys residue they contain.

Knowledge of the complete kinase profile of an inhibitor provides useful information regarding its potential efficacy and specificity towards certain cell types. For example, one can compare the profile to those of other inhibitors. If a subset of target kinases for a new inhibitor overlaps a subset believed, to be relevant for a known effective inhibitor, the new inhibitor should exhibit similar activities and effects. Although the resorcylic acid lactones useful in the methods of the present invention have a unique kinase inhibition profile, certain subsets of the target kinases overlap with subsets inhibited by other effective kinase inhibitors. For example, the kinase inhibitor SU11248 is effective at inhibiting AML containing the FLT3 internal tandem duplication mutation (ITD), because it targets the subset of kinases including FLT3 (wild type and ITD), PDGFR, VEGFR and cKIT. Hypothemycin inhibits the same subset of kinases and therefore, as provided by the present invention, is effective at inhibiting AML cells. In one test, described in the Examples below, the GI₅₀ for SU11248 against the AML (FLT3 ITD) cell line MV-4-11 was 12 nM, and hypothemycin had a GI₅₀ of 6 nM.

Certain kinases and kinase pathways are over- or constitutively-active, either due to overproduction of an enzyme early in the pathway or to an amino acid mutation, such that it may be anticipated that inhibition (directly or indirectly through another earlier enzyme in the pathway) can lead to selective inhibition or modulation of a phenotype resulting from the active pathway. For example, B-Raf V599E (V600E) mutants are found in ˜70% of melanomas and ˜20% of colon cancers, and lead to constitutive activation of the ERK pathway necessary for cell proliferation. BAY 43-9006 was originally developed as a Raf inhibitor to inhibit this pathway in melanoma cells. Hypothemycin and the other RALs useful in the methods of the invention irreversibly inhibit two points of the pathway—MEK1/2 and ERK1/2—and therefore should completely inhibit the pathway and shut down signaling downstream of ERK/RSK phosphorylation.

In vitro testing described in the Examples below shows that B-Raf V599E (V600E) mutants are very sensitive to RAL inhibitors. With the melanoma cell line COLO829, hypothemycin has a GI₅₀ of 50 nM, BAY 43-9006 has a GI₅₀ of 6,000 nM, and SU11248 has a GI₅₀ of 7,100 nM. An activated ERK pathway has also been implicated in a broad spectrum of tumors, including breast, colon, ovarian, prostate and pancreas, as evidenced by cell biology studies and effects of MEK1/2 inhibitors. MEK and Raf inhibitors are effective against cells dependent on the ERK/RSK pathway, and the RALs of the invention are effective against these cells as well.

With a reversible inhibitor of a single enzyme, 100% inhibition is very difficult to achieve, whereas an inhibitor that inhibits multiple steps in a pathway can cause almost complete blockage of a pathway. If a kinase profile shows inhibition of two or more consecutive steps in a linear pathway, it may be predicted that the effect of the drug on the overall pathway will be at least additive if not synergistic. RAL inhibitors useful in the methods of the present invention are unique in that they irreversibly inhibit at least two points in the ERK pathway. They also irreversibly inhibit many of the tyrosine kinase mitogen receptors that stimulate the ERK pathway providing a three-point inhibition of a linear pathway, and consequent powerful inhibition of the mitogen-stimulated proliferation pathway. For example, as shown in the Examples below, with the AML cell line MV-4-11 containing a mutant mitogen receptor Flt3 and constitutively active ERK pathway, hypothemycin has a GI₅₀ of 6 nM. Likewise, hypothemycin is a very potent irreversible inhibitor of VEGFR, and treatment of cells requiring VEGFR shuts down VEGFR, MEK and ERK. Moreover, because ERK phosphorylation is required for VEGF secretion, both production in VEGF producing cells and response to VEGF in VEGF responsive cells are inhibited. For these reasons, hypothemycin and the other RALs disclosed herein as capable of forming Michael adducts with protein kinases having the requisite Cys residue are extraordinarily effective inhibitors of angiogenesis. Another example is the treatment of basal cell carcinoma (BCC). In this indication, 90% of BCC tumors over-express PDGFR which drives the ERK pathway and cell proliferation. RALs useful in the methods of the invention inhibit PDGFR and 2 points in the ERK pathway, thus providing 3-point inhibition of the linear pathway.

Most kinase inhibitors are reversible inhibitors; thus, target inhibition is a function of concentration, and complete inhibition requires inhibitor concentrations far exceeding the inhibitory constant Kᵢ. Also, cells require continuous exposure, because once the inhibitor is removed, enzyme activity rapidly returns. The compounds used in the methods of the invention are irreversible inhibitors of protein kinases, but only irreversibly inhibit the targeted kinase subset. Because target inhibition by hypothemycin and the other RALs useful in the methods of the invention is a function of concentration and/or time, complete inhibition can be achieved at low concentrations of inhibitor if duration of exposure is increased. The present invention provides unit dose forms of and methods for administering the RALs of the invention that take advantage of these properties. Thus, in one embodiment, the methods of the invention for treating disease comprise the administration of sufficient compound to provide blood or tumor levels of the compound that are at or below the inhibitory constant, and/or the maintenance of those levels for a sufficient time so that irreversible inhibition of at least 50%, more preferably greater than 90%, such as 99% or 100%, of the target protein kinases is achieved. In one embodiment, the second administration of the drag (in many embodiments, the drug will be administered multiple times to the same patient), is within one to two days after the first administration of the drug, based on replacement of the irreversibly inhibited kinase by de novo synthesis.

For example, as shown in the Examples below, the ERK pathway in the B-Raf V599E (V600E) cell line COLO829 (and others cells with the BRAF mutation examined) is completely shut down after a 10 min. exposure to hypothemycin at concentrations several-fold lower than K_{d} for the enzyme. Moreover, removal of the inhibitor is not accompanied by immediate regeneration of activity; rather, phosphorylated active ERK is absent for many hours (˜24 hr), and its return apparently requires new enzyme synthesis. Thus, the present invention provides methods for administering these compounds to reduce toxicity to normal cells. In one embodiment, the compound is administered until the target kinase activities are completely inhibited, as determined by measurements taken from a tumor or other cancer cell or tissue. At this point, administration can be stopped without loss of treatment effect and re-initiated only after a significant level of target kinase activity has returned.

In one embodiment, the compounds useful in the methods and contained in the pharmaceutical compositions of the invention have the following general structure I

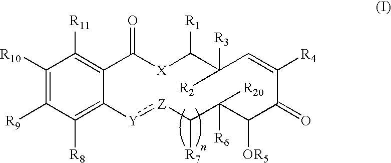

wherein
R₁ is hydrogen or an optionally substituted aliphatic, optionally substituted cycloaliphatic, optionally substituted heterocycloaliphatic, optionally substituted aryl, or optionally substituted heteroaryl moiety;
R₂ and R₃ are each independently hydrogen, halogen, hydroxyl, protected hydroxyl, or an optionally substituted aliphatic, optionally substituted cycloaliphatic, optionally substituted heterocycloaliphatic, optionally substituted aryl or optionally substituted heteroaryl moiety; or R₁ and R₂, when taken together, form an optionally substituted, saturated or unsaturated cyclic ring of 3 to 8 carbon atoms; or R₁ and R₃, when taken together, form an optionally substituted, saturated or unsaturated cyclic ring of 3 to 8 carbon atoms;
R₄ is hydrogen or halogen;
R₅ is hydrogen, C₂ to C₅ alkyl, an oxygen protecting group or a prodrug moiety;
R₆ is hydrogen, hydroxyl, or protected hydroxyl;
n is 0, 1, or 2;
R₇ is, for each occurrence, independently hydrogen, hydroxyl, or protected hydroxyl;
R₈ is hydrogen, halogen, hydroxyl, protected hydroxyl, alkoxy, or an aliphatic moiety optionally substituted with hydroxyl, protected hydroxyl, SR₁₂, or NR₁₂R₁₃;
R₉ is hydrogen, halogen, hydroxyl, protected hydroxyl, OR₁₂, SR₁₂, NR₁₂R₁₃,
   —X₁(CH₂)ₚX₂—R₁₄, or is alkyl optionally substituted with hydroxyl, protected hydroxyl, halogen, amino, protected amino, or —X₁(CH₂)ₚX₂—R₁₄;
   wherein
   R₁₂ and R₁₃ are, independently for each occurrence, hydrogen or an optionally substituted aliphatic, optionally substituted cycloaliphatic, optionally substituted heterocycloaliphatic, optionally substituted aryl, or optionally substituted heteroaryl moiety or an N or S protecting group, or R₁₂ and R₁₃, taken together form a saturated or unsaturated cyclic ring containing 1 to 4 carbon atoms and 1 to 3 nitrogen or oxygen atoms; each of R₁₂ and R₁₃ being optionally substituted with one or more occurrences of hydroxyl, protected hydroxyl, alkoxy, amino, protected amino, —NH(alkyl), aminoalkyl, or halogen;
   X₁ and X₂ are each independently absent, oxygen, NH, or —N(alkyl), or wherein X₂—R₁₄ together are N₃ or are a heterocycloaliphatic moiety;
   p is an integer from 2 to 10, inclusive; and
   R₁₄ is hydrogen or an aryl, heteroaryl, alkylaryl, or alkylheteroaryl moiety, or is —(C═O)NHR₁₅, —(C═O)OR₁₅, or —(C═O)R₁₅, wherein each occurrence of R₁₅ is independently hydrogen or an aliphatic, cycloaliphatic, heterocycloaliphatic, aryl, or heteroaryl moiety; or R₁₄ is —SO₂(R₁₆), wherein R₁₆ is an aliphatic moiety; wherein one or more of R₁₄, R₁₅, and R₁₆ is optionally substituted with one or more occurrences of hydroxyl, protected hydroxyl, alkoxy, amino, protected amino, —NH(alkyl), aminoalkyl, or halogen;
or R₈ and R₉, when taken together, form a saturated or unsaturated cyclic ring containing 1 to 4 carbon atoms and 1 to 3 nitrogen or oxygen atoms, said ring being optionally substituted with hydroxyl, protected hydroxyl, alkoxy, amino, protected amino, —NH(alkyl), aminoalkyl, or halogen;
R₁₀ is hydrogen, hydroxyl, alkoxy, hydroxyalkyl, halogen, or protected hydroxyl;
R₁₁ is hydrogen, hydroxyl, protected hydroxyl, amino, or protected amino;
R₂₀ is hydrogen, or R₂₀ and R₂ combine to form a bond;
X is absent or is O, NH, N-alkyl, CH₂, or S;
Y and Z are connected by a single or double bond, with Y being CHR₁₇, O, C═O, CR₁₇, or NR₁₇ and with Z being CHR₁₈, O, C═O, CR₁₈, or NR₁₈;
   wherein R₁₇ and R₁₈ is are, independently for each occurrence, hydrogen or an optionally substituted aliphatic moiety, or R₁₇ and R₁₈ taken together are —O—, —CH₂— or —NR₁₉—, wherein R₁₉ is hydrogen or alkyl;
      and the pharmaceutically acceptable salts and derivatives thereof.

Preferably, in compounds according to formula I, at least one of the following provisions apply: (i) R₆ is hydrogen or hydroxyl, (ii) n is 1, (iii) R₈ is other than halogen, (iv) R₁₀ is hydrogen, and (v) R₁₁ is other than protected hydroxyl.

In a preferred embodiment, the compound has a structure according to formula Ia,

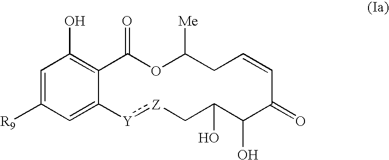

wherein
R₉ is hydrogen, halogen, hydroxyl, protected hydroxyl, OR₁₂, SR₁₂, NR₁₂R₁₃,
   —X₁(CH₂)ₚX₂—R₁₄, or is alkyl optionally substituted with hydroxyl, protected hydroxyl, halogen, amino, protected amino, or —X₁(CH₂)ₚX₂—R₁₄;
   wherein
   R₁₂ and R₁₃ are, independently for each occurrence, hydrogen or an optionally substituted aliphatic, optionally substituted cycloaliphatic, optionally substituted heterocycloaliphatic, optionally substituted aryl, or optionally substituted heteroaryl moiety or an N or S protecting group, or R₁₂ and R₁₃, taken together form a saturated or unsaturated cyclic ring containing 1 to 4 carbon atoms and 1 to 3 nitrogen or oxygen atoms; each of R₁₂ and R₁₃ being optionally substituted with one or more occurrences of hydroxyl, protected hydroxyl, alkoxy, amino, protected amino, —NH(alkyl), aminoalkyl, or halogen;
   X₁ and X₂ are each independently absent, oxygen, NH, or —N(alkyl), or wherein X₂—R₁₄ together are N₃ or are a heterocycloaliphatic moiety;
   p is an integer from 2 to 10, inclusive; and
   R₁₄ is hydrogen or an aryl, heteroaryl, alkylaryl, or alkylheteroaryl moiety, or is —(C═O)NHR₁₅, —(C═O)OR₁₅, or —(C═O)R₁₅, wherein each occurrence of R₁₅ is independently hydrogen or an aliphatic, cycloaliphatic, heterocycloaliphatic, aryl, or heteroaryl moiety; or R₁₄ is —SO₂(R₁₆), wherein R₁₆ is an aliphatic moiety; wherein one or more of R₁₄, R₁₅, and R₁₆ is optionally substituted with one or more occurrences of hydroxyl, protected hydroxyl, alkoxy, amino, protected amino, —NH(alkyl), aminoalkyl, or halogen; and
Y and Z are connected by a single or double bond, with Y being CHR₁₇, and with Z being CHR₁₈; wherein R₁₇ and R₁₈ are hydrogen, or R₁₇ and R₁₈ taken together are —O—.

In a preferred embodiment of compounds according to formula Ia, OR₁₂ in R₉ is other than OMe.

In another preferred embodiment, the compound has a structure according to formula Ib

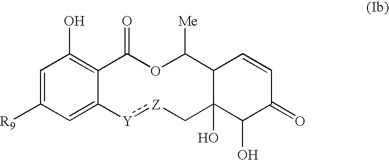

wherein
R₉ is hydrogen, halogen, hydroxyl, protected hydroxyl, OR₁₂, SR₁₂, NR₁₂R₁₃,
   —X₁(CH₂)ₚX₂—R₁₄, or is alkyl optionally substituted with hydroxyl, protected hydroxyl, halogen, amino, protected amino, or —X₁(CH₂)ₚX₂—R₁₄;
   wherein
   R₁₂ and R₁₃ are, independently for each occurrence, hydrogen or an optionally substituted aliphatic, optionally substituted cycloaliphatic, optionally substituted heterocycloaliphatic, optionally substituted aryl, or optionally substituted heteroaryl moiety or an N or S protecting group, or R₁₂ and R₁₃, taken together form a saturated or unsaturated cyclic ring containing 1 to 4 carbon atoms and 1 to 3 nitrogen or oxygen atoms; each of R₁₂ and R₁₃ being optionally substituted with one or more occurrences of hydroxyl, protected hydroxyl, alkoxy, amino, protected amino, —NH(alkyl), aminoalkyl, or halogen;
   X₁ and X₂ are each independently absent, oxygen, NH, or —N(alkyl), or wherein X₂—R₁₄ together are N₃ or are a heterocycloaliphatic moiety;
   p is an integer from 2 to 10, inclusive; and
   R₁₄ is hydrogen or an aryl, heteroaryl, alkylaryl, or alkylheteroaryl moiety, or is —(C═O)NHR₁₅, —(C═O)OR₁₅, or —(C═O)R₁₅, wherein each occurrence of R₁₅ is independently hydrogen or an aliphatic, cycloaliphatic, heterocycloaliphatic, aryl, or heteroaryl moiety; or R₁₄ is —SO₂(R₁₆), wherein R₁₆ is an aliphatic moiety; wherein one or more of R₁₄, R₁₅, and R₁₆ is optionally substituted with one or more occurrences of hydroxyl, protected hydroxyl, alkoxy, amino, protected amino, —NH(alkyl), aminoalkyl, or halogen; and
Y and Z are connected by a single or double bond, with Y being CHR₁₇, and with Z being CHR₁₈; wherein R₁₇ and R₁₈ are hydrogen, or R₁₇ and R₁₈ taken together are —O—.

In another preferred embodiment, the compound has a structure according to formula Ic

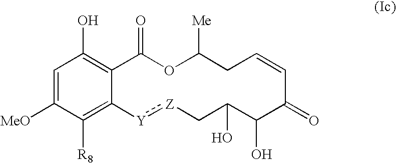

wherein
R₈ is hydrogen, halogen, hydroxyl, protected hydroxyl, alkoxy, or an aliphatic moiety optionally substituted with hydroxyl, protected hydroxyl, SR₁₂, or NR₁₂R₁₃; and
Y and Z are connected by a single or double bond, with Y being CHR₁₇, and with Z being CHR₁₈; wherein R₁₇ and R₁₈ are hydrogen, or R₁₇ and R₁₈ taken together are —O—.

In a preferred embodiment of compounds according to formula Ic, R₈ is other than hydrogen or halogen.

In another preferred embodiment, the compound has a structure according to formula Id

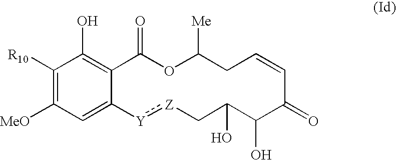

wherein
R₁₀ is hydrogen, hydroxyl, alkoxy, hydroxyalkyl, halogen, or protected hydroxyl; and
Y and Z are connected by a single or double bond, with Y being CHR₁₇, and with Z being CHR₁₈; wherein R₁₇ and R₁₈ is are hydrogen, or R₁₇ and R₁₈ taken together are —O—.

In another preferred embodiment, the compound has a structure according to formula Ie

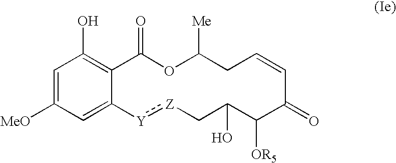

R₅ is hydrogen, C₂ to C₅ alkyl, an oxygen protecting group or a prodrug moiety; and
Y and Z are connected by a single or double bond, with Y being CHR₁₇, and with Z being CHR₁₈; wherein R₁₇ and R₁₈ are hydrogen, or R₁₇ and R₁₈ is taken together are —O—.

In a preferred embodiment of compounds according to formula Ie, R₅ is other than hydrogen.

In another preferred embodiment, the compound has a structure according to formula If:

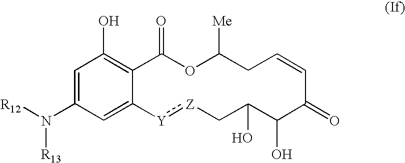

wherein
R₁₂ and R₁₃ are, independently for each occurrence, hydrogen or an optionally substituted aliphatic, optionally substituted cycloaliphatic, optionally substituted heterocycloaliphatic, optionally substituted aryl, or optionally substituted heteroaryl moiety or an N or S protecting group, or R₁₂ and R₁₃, taken together form a saturated or unsaturated cyclic ring containing 1 to 4 carbon atoms and 1 to 3 nitrogen or oxygen atoms; each of R₁₂ and R₁₃ being optionally substituted with one or more occurrences of hydroxyl, protected hydroxyl, alkoxy, amino, protected amino, —NH(alkyl), aminoalkyl, or halogen;
Y and Z are connected by a single or double bond, with Y being CHR₁₇, and with Z being CHR₁₈; wherein R₁₇ and R₁₈ are hydrogen, or R₁₇ and R₁₈ taken together are —O—.

In another preferred embodiment, the compound has a structure according to formula Ig:

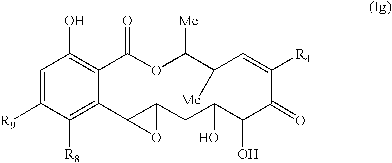

wherein

### R₄ is H or F;

### R₈ is H; and

R₉ is selected from the group consisting of

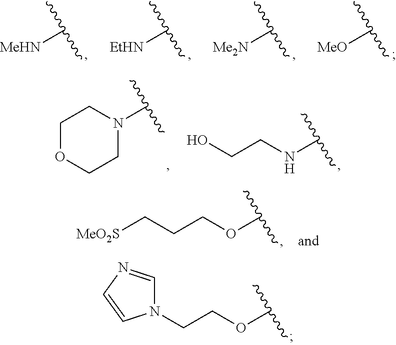

or R₈ and R₉ combine to form

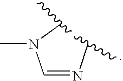

“Aliphatic” means a straight- or branched-chain, saturated or unsaturated, non-aromatic hydrocarbon moiety having the specified number of carbon atoms (e.g., as in “C₃ aliphatic,” “C₁-C₅ aliphatic,” or “C₁ to C₅ aliphatic,” the latter two phrases being synonymous for an aliphatic moiety having from 1 to 5 carbon atoms) or, where the number of carbon atoms is not specified, from 1 to 4 carbon atoms. Those skilled in the art will understand that an unsaturated aliphatic moiety necessarily comprises at least two carbon atoms.

“Alkyl” means a saturated aliphatic moiety, with the same convention for designating the number of carbon atoms being applicable. By way of illustration, C₁-C₄ alkyl moieties include, but are not limited to, methyl, ethyl, propyl, isopropyl, isobutyl, t-butyl, 1-butyl, 2-butyl, and the like.

“Alkenyl” means an aliphatic moiety having at least one carbon-carbon double bond, with the same convention for designating the number of carbon atoms being applicable. By way of illustration, C₂-C₄ alkenyl moieties include, but are not limited to, ethenyl (vinyl), 2-propenyl (allyl or prop-2-enyl), cis-1-propenyl, trans-1-propenyl, E- (or Z-)2-butenyl, 3-butenyl, 1,3-butadienyl (but-1,3-dienyl) and the like.

“Alkynyl” means an aliphatic moiety having at least one carbon-carbon triple bond, with the same convention for designating the number of carbon atoms being applicable. By way of illustration, C₂-C₄ alkynyl groups include ethynyl (acetylenyl), propargyl (prop-2-ynyl), 1-propynyl, but-2-ynyl, and the like.

“Cycloaliphatic” means a saturated or unsaturated, non-aromatic hydrocarbon moiety having from 1 to 3 rings and each ring having from 3 to 8 (preferably from 3 to 6) carbon atoms. “Cycloalkyl” means a cycloaliphatic moiety in which each ring is saturated. “Cycloalkenyl” means a cycloaliphatic moiety in which at least one ring has at least one carbon-carbon double bond. “Cycloalkynyl” means a cycloaliphatic moiety in which at least one ring has at least one carbon-carbon triple bond. By way of illustration, cycloaliphatic moieties include, but are not limited to, cyclopropyl, cyclobutyl, cyclopentyl, cyclopentenyl, cyclohexyl, cyclohexenyl, cycloheptyl, cyclooctyl, and adamantyl. Preferred cycloaliphatic moieties are cycloalkyl ones, especially cyclopropyl, cyclobutyl, cyclopentyl, and cyclohexyl.

“Heterocycloaliphatic” means a cycloaliphatic moiety wherein, in at least one ring thereof, up to three (preferably 1 to 2) carbons have been replaced with a heteroatom independently selected from N, O, or S, where the N and S optionally may be oxidized and the N optionally may be quaternized. Similarly, “heterocycloalkyl,” “heterocycloalkenyl,” and “heterocycloalkynyl” means a cycloalkyl, cycloalkenyl, or cycloalkynyl moiety, respectively, in which at least one ring thereof has been so modified. Exemplary heterocycloaliphatic moieties include aziridinyl, azetidinyl, 1,3-dioxanyl, oxetanyl, tetrahydrofuryl, pyrrolidinyl, piperidinyl, piperazinyl, tetrahydropyranyl, tetrahydro-thiopyranyl, tetrahydrothiopyranyl sulfone, morpholinyl, thiomorpholinyl, thiomorpholinyl sulfoxide, thiomorpholinyl sulfone, 1,3-dioxolanyl, tetrahydro-1,1-dioxothienyl, 1,4-dioxanyl, thietanyl, and the like.

“Alkoxy”, “aryloxy”, “alkylthio”, and “arylthio” mean —O(alkyl), —O(aryl), —S(alkyl), and —S(aryl), respectively. Examples are methoxy, phenoxy, methylthio, and phenylthio, respectively.

“Halogen” or “halo” means fluorine, chlorine, bromine or iodine.

“Aryl” means a hydrocarbon moiety having a mono-, bi-, or tricyclic ring system wherein each ring has from 3 to 7 carbon atoms and at least one ring is aromatic. The rings in the ring system may be fused to each other (as in naphthyl) or bonded to each other (as in biphenyl) and may be fused or bonded to non-aromatic rings (as in indanyl or cyclohexylphenyl). By way of further illustration, aryl moieties include, but are not limited to, phenyl, naphthyl, tetrahydronaphthyl, indanyl, biphenyl, phenanthryl, anthracenyl, and acenaphthyl.

“Heteroaryl” means a moiety having a mono-, bi-, or tricyclic ring system wherein each ring has from 3 to 7 carbon atoms and at least one ring is an aromatic ring containing from 1 to 4 heteroatoms independently selected from N, O, or S, where the N and S optionally may be oxidized and the N optionally may be quaternized. Such at least one heteroatom containing aromatic ring may be fused to other types of rings (as in benzofuranyl or tetrahydro isoquinolyl) or directly bonded to other types of rings (as in phenylpyridyl or 2-cyclopentylpyridyl). By way of further illustration, heteroaryl moieties include pyrrolyl, furanyl, thiophenyl (thienyl), imidazolyl, pyrazolyl, oxazolyl, isoxazolyl, thiazolyl, isothiazolyl, triazolyl, tetrazolyl, pyridyl, N-oxopyridyl, pyridazinyl, pyrimidinyl, pyrazinyl, quinolinyl, isoquinolynyl, quinazolinyl, cinnolinyl, quinozalinyl, naphthyridinyl, benzofuranyl, indolyl, benzothiophenyl, benzimidazolyl, benzotriazolyl, dibenzofuranyl, carbazolyl, dibenzothiophenyl, acridinyl, and the like.

Where it is indicated that a moiety may be substituted, such as by use of “substituted or unsubstituted” or “optionally substituted” phrasing as in “substituted or unsubstituted C₁-C₅ alkyl” or “optionally substituted heteroaryl,” such moiety may have one or more independently selected substituents, preferably one to five in number, more preferably one or two in number. Substituents and substitution patterns can be selected by one of ordinary skill in the art, having regard for the moiety to which the substituent is attached, to provide compounds that are chemically stable and that can be synthesized by techniques known in the art as well as the methods set forth herein.

“Arylalkyl”, (heterocycloaliphatic)alkyl”, “arylalkenyl”, “arylalkynyl”, “biarylalkyl”, and the like mean an alkyl, alkenyl, or alkynyl moiety, as the case may be, substituted with an aryl, heterocycloaliphatic, biaryl, etc., moiety, as the case may be, with the open (unsatisfied) valence at the alkyl, alkenyl, or alkynyl moiety, for example as in benzyl, phenethyl, N-imidazoylethyl, N-morpholinoethyl, and the like. Conversely, “alkylaryl”, “alkenylcycloalkyl”, haloheteroaryl, and the like mean an aryl, cycloalkyl, heteroaryl, etc., moiety, as the case may be, substituted with an alkyl, alkenyl, halo, etc., moiety, as the case may be, for example as in methylphenyl (tolyl) or allylcyclohexyl. “Hydroxyalkyl”, “haloalkyl”, “aminoalkyl”, “alkylaryl”, “cyanoaryl”, and the like mean an alkyl, aryl, etc., moiety, as the case may be, substituted with the identified substituent (hydroxyl, halo, amino, etc., as the case may be). By way of illustration, permissible substituents include, but are not limited to, alkyl (especially methyl or ethyl), alkenyl (especially allyl), alkynyl, aryl, heteroaryl, cycloaliphatic, heterocycloaliphatic, halo (especially fluoro), haloalkyl (especially trifluoromethyl), hydroxyl, hydroxyalkyl (especially hydroxyethyl), cyano, nitro, alkoxy, —O(hydroxyalkyl), —O(haloalkyl) (especially —OCF₃), —O(cycloalkyl), —O(heterocycloalkyl), —O(aryl), alkylthio, arylthio, ═O, ═NH, ═N(alkyl), ═NOH, ═NO(alkyl), —C(═O)(alkyl), —C(═O)H, —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, azido, —NH₂, —NH(alkyl), —N(alkyl)₂, —NH(aryl), —NH(hydroxyalkyl), —NHC(═O)(alkyl), —NHC(═O)H, —NHC(═O)NH₂, —NHC(═O)NH(alkyl), —NHC(═O)N(alkyl)₂, —NHC(═NH)NH₂, —OSO₂(alkyl), —SH, —S(alkyl), —S(aryl), —S(cycloalkyl), —S(═O)alkyl, —SO₂(alkyl), —SO₂NH₂, —SO₂NH(alkyl), —SO₂N(alkyl)₂, and the like.

Where the moiety being substituted is an aliphatic moiety, preferred substituents are aryl, heteroaryl, cycloaliphatic, heterocycloaliphatic, halo, hydroxyl, cyano, nitro, alkoxy, —O(hydroxyalkyl), —O(haloalkyl), —O(cycloalkyl), —O(heterocycloalkyl), —O(aryl), alkylthio, arylthio, ═O, ═NH, ═N(alkyl), ═NOH, ═NO(alkyl), —CO₂H, —C(═O)NHOH, —C(═O)O(alkyl), —C(═O)O(hydroxyalkyl), —C(═O)NH₂, C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, azido, —NH₂, —NH(alkyl), —N(alkyl)₂, —NH(aryl), —NH(hydroxyalkyl), —NHC(═O)(alkyl), —NHC(═O)H, —NHC(═O)NH₂, —NHC(═O)NH(alkyl), —NHC(═O)N(alkyl)₂, —NHC(═NH)NH₂, —OSO₂(alkyl), —SH, —S(alkyl), —S(aryl), —S(cycloalkyl), —S(═O)alkyl, —SO₂(alkyl), —SO₂NH₂, —SO₂NH(alkyl), and —SO₂N(alkyl)₂. More preferred substituents are halo, hydroxyl, cyano, nitro, alkoxy, —O(aryl), ═O, ═NOH, ═NO(alkyl), —OC(═O)(alkyl), —OC(═O)O(alkyl), —OC(═O)NH₂, —OC(═O)NH(alkyl), —OC(═O)N(alkyl)₂, azido, —NH₂, —NH(alkyl), —N(alkyl)₂, —NH(aryl), —NHC(═O)(alkyl), —NHC(═O)H, —NHC(═O)NH₂, —NHC(═O)NH(alkyl), —NHC(═O)N(alkyl)₂, and —NHC(═NH)NH₂.

Where the moiety being substituted is a cycloaliphatic, heterocycloaliphatic, aryl, or heteroaryl moiety, preferred substituents are alkyl, alkenyl, alkynyl, halo, haloalkyl, hydroxyl, hydroxyalkyl, cyano, nitro, alkoxy, —O(hydroxyalkyl), —O(haloalkyl), —O(cycloalkyl), —O(heterocycloalkyl), —O(aryl), alkylthio, arylthio, —C(═O)(alkyl), —C(═O)H, —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, azido, —NH₂, —NH(alkyl), —N(alkyl)₂, —NH(aryl), —NH(hydroxyalkyl), —NHC(═O)(alkyl), —NHC(═O)H, —NHC(═O)NH₂, —NHC(═O)NH(alkyl), —NHC(═O)N(alkyl)₂, NHC(═NH)NH₂, —OSO₂(alkyl), —SH, —S(alkyl), —S(aryl), —S(cycloalkyl), —S(═O)alkyl, —SO₂(alkyl), —SO₂NH₂, —SO₂NH(alkyl), and —SO₂N(alkyl)₂. More preferred substituents are alkyl, alkenyl, halo, haloalkyl, hydroxyl, hydroxyalkyl, cyano, nitro, alkoxy, —O(hydroxyalkyl), —C(═O)(alkyl), —C(═O)H, —CO₂H, —C(═O)NHOH, C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, —NH₂, —NH(alkyl), —N(alkyl)₂, —NH(aryl), —NHC(═O)(alkyl), —NHC(═O)H, NHC(═O)NH₂, —NHC(═O)NH(alkyl), —NHC(═O)N(alkyl)₂, and —NHC(═NH)NH₂.

Where a range is stated, as in “C₁ to C₅ alkyl” or “5 to 10%,” such range includes the end points of the range.

Unless particular stereoisomers are specifically indicated (e.g., by a bolded or dashed bond at a relevant stereocenter in a structural formula, by depiction of a double bond as having E or Z configuration in a structural formula, or by use of stereochemistry-designating nomenclature), all stereoisomers are included within the scope of the invention, as pure compounds as well as mixtures thereof. Unless otherwise indicated, individual enantiomers, diastereomers, geometrical isomers, and combinations and mixtures thereof are all encompassed by the present invention. Polymorphic crystalline forms and solvates are also encompassed within the scope of this invention.

“Pharmaceutically acceptable salt” means a salt of a compound suitable for pharmaceutical formulation as a salt. Where a compound has one or more basic functionalities, the salt can be an acid addition salt, such as a sulfate, hydrobromide, tartrate, mesylate, maleate, citrate, phosphate, acetate, pamoate (embonate), hydroiodide, nitrate, hydrochloride, lactate, methylsulfate, fumarate, benzoate, succinate, mesylate, lactobionate, suberate, tosylate, and the like. Where a compound has one or more acidic moieties, the salt can be a salt such as a calcium salt, potassium salt, magnesium salt, meglumine salt, ammonium salt, zinc salt, piperazine salt, tromethamine salt, lithium salt, choline salt, diethylamide salt, 4-phenylcyclohexylamine salt, benzathine salt, sodium salt, tetramethylammonium salt, and the like.

The present invention includes within its scope prodrugs of the compounds of this invention. Such prodrugs are in general functional derivatives of the compounds that are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to a subject in need thereof. Conventional procedures for the selection and preparation of suitable prodrug derivatives are described, for example, in Wermuth, “Designing Prodrugs and Bioprecursors,” in Wermuth, ed., The Practice of Medicinal Chemistry, 2nd Ed., pp. 561-586 (Academic Press 2003), the disclosure of which is incorporated herein by reference. Prodrugs include esters that hydrolyze in vivo (for example in the human body) to produce a compound of this invention or a salt thereof. Suitable ester groups include, without limitation, those derived from pharmaceutically acceptable aliphatic carboxylic acids, particularly alkanoic, alkenoic, cycloalkanoic and alkanedioic acids, in which each alkyl or alkenyl moiety preferably has no more than six carbon atoms. Illustrative esters include but are not limited to formates, acetates, propionates, butyrates, acrylates, citrates, succinates, and ethylsuccinates.

In one important embodiment, compounds of the invention include prodrug esters of the resorcylic acid lactones useful in the methods of the invention suitable for oral administration. In one embodiment, these prodrugs are amino acid esters (including but not limited to dimethylglycine esters and valine esters) of the resorcylic acid lactones useful in the methods of the invention.

“Protecting group” means a moiety that temporarily blocks a particular functional moiety, e.g., O, S, or N, so that a reaction can be carried out selectively at another reactive site in a multifunctional compound. In preferred embodiments, a protecting group (a) reacts selectively in good yield to give a protected substrate that is stable to the projected reactions; (b) can be selectively removed in good yield by readily available, preferably nontoxic reagents that do not attack the other functional groups; (c) forms an easily separable derivative (more preferably without the generation of new stereogenic centers); and (d) has a minimum of additional functionality to avoid further sites of reaction. “Oxygen protecting group” means a protective group attached to oxygen and includes, but is not limited to methyl ethers, substituted methyl ethers (e.g., MOM (methoxymethyl ether), MTM (methylthiomethyl ether), BOM (benzyloxymethyl ether), PMBM or MPM (p-methoxybenzyloxymethyl ether)), substituted ethyl ethers, substituted benzyl ethers, silyl ethers (e.g., TMS (trimethylsilyl ether), TES (triethylsilylether), TIPS (triisopropylsilyl ether), TBDMS (t-butyldimethylsilyl ether), tribenzyl silyl ether. TBDPS (t-butyldiphenyl silyl ether)), esters (e.g., formate, acetate, benzoate (Bz), trifluoroacetate, dichloroacetate), carbonates, cyclic acetals and ketals. “Nitrogen protecting group” means a protecting group attached to an amine nitrogen and includes, but is not limited to, carbamates (e.g., methyl, ethyl and substituted ethyl carbamates (e.g., Troc)) amides, cyclic imide derivatives, N-alkyl and N-aryl amines, imine derivatives, and enamine derivatives. Many examples of protecting groups can be found in Greene and Wuts, Protective Groups in Organic Synthesis; 3rd edition, pp. 17-245 (John Wiley & Sons, New York, 1999), along with teachings regarding their manner of use; the disclosure of which is incorporated herein by reference. Thus, “protected hydroxyl” means a hydroxyl group in which the hydrogen has been replaced by an oxygen protecting group and “protected amine” means a primary or secondary amine group in which a hydrogen has been replaced by a nitrogen protecting group.

Analogs and derivatives of the compounds encompassed by the above structure that retain the critical cis double bond conjugated to a carbonyl (or a bioisostere) at positions 5-7 are also useful in the methods of the invention. Generally, any compound, whether a resorcylic acid lactone or derivative or other compound, that is capable of forming a Michael adduct with the critical Cys residue can be used in one or more of the methods of the invention. For example, a compound of the invention can be designed using crystal structures, such that the compound consists essentially of a Michael acceptor appended to the appropriate position of a known inhibitor of one of these enzymes. The resulting compound can form a reversible complex with the enzyme, after which covalent bond formation would occur.

Thus, compounds useful in the methods of the invention specifically inhibit protein kinases having a Cys residue in the ATP-binding site located between the two and adjacent to one of the conserved Asp residues and, importantly, have negligible inhibitory activity against protein kinases lacking this Cys at this position in the ATP-binding site. Thus, such can be used to inhibit particular protein kinases specifically, which provides important new methods for treating human diseases. Also, because such protein kinases exist in multiple signaling pathways, the compounds useful in the methods of the invention can provide the multiple pathway blocking effect required for therapeutic activity.

Protein kinases containing this critical Cys include but are not limited to AAK1, APEG1 splice variant with kinase domain (SPEG), BMP2K (BIKE), CDKL1, CDKL2, CDKL3, CDKL4, CDKL5 (STK9), ERK1 (MAPK3), ERK2 (MAPK1), FLT3, GAK, GSK3A, GSK3B, KIT (cKIT), MAP3K14 (NIK), MAP3K7 (TAK1), MAPK15 (ERK8), MAPKAPK5 (PRAK), MEK1 (MKK1, MAP2K1), MEK2 (MKK2, MAP2K2), MEK3 (MKK3, MAP2K3), MEK4 (MKK4, MAP2K4), MEK5 (MKK5, MAP2K5), MEK6 (MKK6, MAP2K6), MEK7 (MKK7, MAP2K7), MKNK1 (MNK1), MKNK2 (MNK2, GPRK7), NLK, PDGFR alpha, PDGFR beta, PRKD1 (PRKCM), PRKD2, PRKD3 (PRKCN), PRPF4B (PRP4K), RPS6KA1 (RSK1, MAPKAPK1A), RPS6KA2 (RSK3, MAPKAP1B), RPS6KA3 (RSK2, MAPKAP1C), RPS6KA6 (RSK4), STK36 (FUSED_STK), STYK1, TGFBR2, TOPK, VEGFR1 (FLT1), VEGFR2 (KDR), VEGFR3 (FLT4) and ZAK.

The methods of the present invention include the administration of RALs or derivatives that can achieve multiple signaling pathway inhibition by inhibiting specific protein kinases in different cell signaling pathways. This type of inhibition can be desirable or even necessary to achieve a desired effect, as illustrated above with GLEEVEC. Another illustrative example is the inhibition of Hsp90 by inhibitors like geldanamycin, 17-AAG, and 17-DMAG. This inhibition affects multiple pathways, because inhibition of Hsp90 results in degradation/inhibition of multiple client protein kinases from multiple cell signaling pathways.

It is difficult, however, to design an inhibitor that inhibits multiple protein kinases specifically, without inhibiting many kinases generally. Likewise, it is difficult, even if one has identified a protein kinase inhibitor, to predict which of the over 500 other protein kinases the inhibitor will inhibit. In contrast, the core structure of the compounds useful in the methods of the present invention, the enone or alpha, beta-unsaturated ketone moiety capable of Michael adduct formation with the critical Cys in the protein kinase provides exquisite specificity and improved therapeutic results. In one embodiment, these compounds of the invention contain the enone moiety at positions 5-7 in a resorcylic acid lactone structure. With such compounds, one can inhibit a specific subset of all kinases predictably. Compounds of the invention also include the large number of compounds that are structural modifications of the core structure, such that one can select a particular inhibitor that exhibits the balance of kinase inhibition within the specific set of kinases that is desired for the therapeutic indication.

Multiple protein kinase inhibition can inhibit (a) different branches of a network, creating the potential to inhibit an entire network, or (b) different kinases along a single linear branch of a network, or (c) both. Multiple protein kinase inhibition of these types provides an additive inhibitory effect over compounds that inhibit only a single kinase and have the potential to create synergistic inhibition. Certain resorcylic acid lactone inhibitors are useful in illustrating how the methods of the invention can encompass either or both approaches. For example, these inhibitors inhibit the ERK signaling pathway and the INK signaling pathway, thus affecting different, balanced signaling pathways important in both cell proliferation and inflammation and illustrating the network inhibition approach.

Certain resorcylic acid lactone inhibitors useful in the methods of the invention also inhibit multiple enzymes in single pathways, the synergistic pathway inhibition approach. For example, certain resorcylic acid lactone compounds inhibit MEK1/2 and ERK1/2. Such inhibitors and other compounds of the invention can be administered to achieve clinically relevant inhibition of a disease process, even if their potency against any one particular protein kinase is not extremely high.

For example, if one assumes an inhibitor is equally potent for activated (i.e. phosphorylated) forms of both enzymes, then the concentration of that inhibitor necessary to inhibit 50% of MEK1/2 results in formation of only 50% of the phosphorylated form of ERK1/2 (relative to no inhibition). If, at the same concentration, the inhibitor simultaneously inhibits 50% of activated ERK1/2, then the pathway is inhibited by 75%, a synergistic inhibition of the pathway. Further, certain compounds useful in the methods of the invention inhibit not only multiple kinases in the ERK pathway but also inhibit VEGFR, which, when activated, causes ERK pathway activation. If an inhibitor has the same potency against all three enzymes, then the signaling pathway (the target of the inhibitor for anti-proliferative effects) from VEGFR through ERK1/2 is inhibited by 87.5% at a concentration that inhibits any single enzyme by only 50%.

This multiple protein kinase inhibition is illustrated in one embodiment of the present invention relating to therapeutic methods that involve the inhibition of PDGFRB, PDGFRA, and KIT to achieve the desired therapeutic effect. These targets are inhibited by GLEEVEC, which has therapeutic value in the treatment of chronic myelomonocytic leukemia and glioblastoma multiforme as well as GIST and metastatic GIST (GLEEVEC also inhibits Bcr-Abl, which is not susceptible to Michael adduct formation with the compounds useful in the methods of this invention). Thus, the compounds and pharmaceutical compositions useful in the methods of the invention have therapeutic application against these diseases. Importantly, however, the binding of the compounds useful in the invention to the protein kinase is such that mutations in the protein kinase that confer GLEEVEC resistance do not confer resistance to the inventive compounds. Thus, the methods of the invention include methods for treating GLEEVEC resistant disease conditions, including the GLEEVEC resistant forms of the cancers for which GLEEVEC is administered. The methods of the invention also include methods for treating other cancer indications and diseases, as discussed in the following sections, each focused on a particular cancer or other disease indication.

### Gastrointestinal Stromal Tumors

Gastrointestinal stromal tumors (GISTs) are found predominantly in the stomach (60%) and small intestine (25%) but also occur at lower frequency in the rectum, esophagus and other locations. GISTs were often misidentified in the past, so it is difficult to get an accurate historical picture of their incidence. There are estimated to be approximately 5000 new cases each year in the United States (www.orpha.net/data/patho/GB/uk-GIST.pdf). Approximately 95% of GISTs stain positive for c-Kit immunohistochemically and up to 85% of GISTs harbor activating mutations of the c-Kit tyrosine kinase (Hirota et al., Science 1998; 279 (5350):577-80). In addition, several kindred groups with heritable activating mutations of c-Kit have been identified (Nishida et al., Nat Genet. 1998; 19 (4):323-4). These families suffer from the development of multiple benign and malignant GISTs. Of the GISTs that were found to be wild-type for c-Kit, approximately 5% harbor mutations in PDGFRA (Heinrich et al., Science 2003; 299 (5607):708-10). Activating mutations of the c-Kit and PDGFRA tyrosine kinases are associated with activation of downstream signaling pathways, including the MEK1/2 and ERK1/2 enzyme pathways. Hypothemycin and its derivatives and analogs as described herein are potent inhibitors of the receptor kinases c-KIT and PDGFR, as well as the sequential MEK1/2 and ERK1/2 in the ERK pathway, and can be administered in accordance with the methods of the invention to patients for the treatment for GIST.

### Acute Myeloid Leukemia

The compounds useful in the methods of the invention also include those that inhibit FLT3, the most common molecular abnormality (mutation) in acute myeloid leukemia (AML). AML is the most common leukemia in adults as well as being the most common form of cancer in children. Approximately 10,000 new cases and 8,000 deaths were caused by AML in 2003 in the United States; about the same number of cases occurred in Europe and Australia. Several kinases have been implicated to have a role in AML. Therapeutic targets for current drugs in clinical trials to treat AML include FLT3, c-KIT and VEGFR. FLT3 plays an important role in normal hematopoiesis and leukemogenesis. It is abnormally activated or up-regulated in 70% to 100% of patients with AML (see Spiekermann et al., Clin. Cancer Res. 2003; 9 (6):2140-50; and Blood 2003; 101 (4): 1494-504). The c-Kit protein kinase has been found at high levels in 60% to 80% of AML patients and is believed to mediate proliferation and anti-apoptotic effects (Heinrich et al., J Clin Oncol 2002; 20 (6): 1692-703). VEGF and VEGFR have been implicated to play a role in bone marrow angiogenesis (Aguayo et al., Blood 2000; 96 (6):2240-5). Bone marrow biopsies of AML patients have shown that changes in VEGF and VEGFR levels parallel changes in micro-vessel density (Kuzu et al., Leuk Lymphoma 2004; 45 (6): 1185-90). VEGF levels appear to correlate inversely to survival in patients with AML (Brunner et al., J. Hematother. Stem Cell Res. 2002; 11 (1): 119-25). Hypothemycin is a potent inhibitor of FLT3, c-KIT, VEGFR and VEGF production (via inhibition of MEK1/2 and ERK1/2 in the ERK pathway), and in accordance with the methods of the present invention, hypothemycin and its derivatives and analogs as described herein can be administered to patients for the treatment for AML.

Thus, the methods of the invention include methods for treating AML. In one embodiment, those methods include the initial step of identifying whether diseased tissue contains cells having a FLT3 mutation indicative of AML or other cancer type. FLT3 mutations occur in AML (˜41% of patients). These mutations include but are not limited to Asp835 in the activation loop, and D835->Y or V or H or E or N, which can be detected in accordance with known procedures.

Cancers Associated with B-Raf Mutations

A specific B-Raf mutation V599E (V600E) is found in 70% of malignant melanomas and about 20% of colon cancers. In one embodiment of the invention, a cancer patient's tumor is biopsied to determine if the tumor cells exhibit the B-Raf mutation characteristic of these ERK pathway dependent cancers, and if the B-Raf mutation is present, then a compound useful in the method of the invention is administered to treat the cancer.

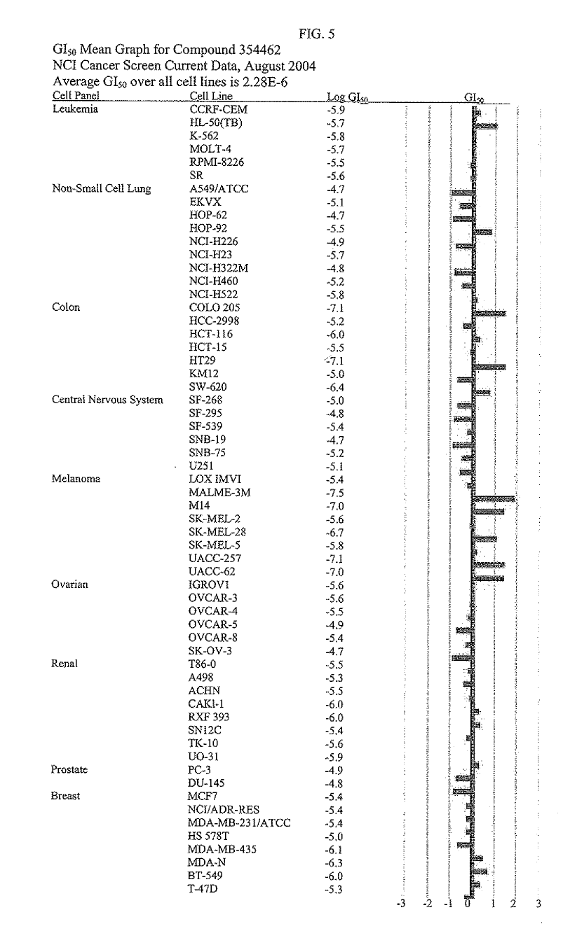

The efficacy of this combined diagnostic/therapeutic method, or “theranostic,” is illustrated in part by the data in FIG. 5. Hypothemycin, a resorcylic acid lactone useful in certain methods of the invention, has been tested against the 60 cell line NCI panel, the results of which, log GI₅₀ values (the amount of drug required to achieve 50% growth reduction) are shown in bar graph form in FIG. 5. Cell lines most sensitive to the compound are depicted with bars pointing to the right from the vertical mean activity. The results show that the sensitive cell lines were derived from B-Raf-dependent cancers having the B-Raf mutation V599E (V600E) with aberrant MAPK signaling pathways involving protein kinases (e.g. MEK1/2, ERK1/2), as can be predicted in view of the teachings herein to be sensitive to hypothemycin due to the presence of the critical Cys residue in these mutant kinases and the presence of the necessary structure for reversible binding and critical Michael adduct formation.

Table 1 presents in tabular form data supporting the utilities of the invention discussed above.

TABLE 1
Sensitivity of B-Raf Mutated Cancer Cells to Kinase Inhibitors

| Cell Line | Kinase Inhibitor (IC₅₀, μM) | | | | |
|---|---|---|---|---|---|
| (cancer type, kinase | | | | BAY | |
| mutation) | Hypothemycin | 5,6-Dihydrohypothemycin | SU11248 | 43-9006 | PD 98059 |
| A549 | 6 | 107 | — | 5.5 | 48 |
| (NSCLC, B-Raf wild- | | | | | |
| type) | | | | | |
| HT29 | 0.1 | 15 | 4.2 | 4.7 | 5.5 |
| (Human colon, B-Raf | | | | | |
| V599E) | | | | | |
| DU4475 | 0.018 | 46 | 4.0 | 3.6 | 56 |
| (Human breast, B-Raf | | | | | |
| V599E) | | | | | |
| WM266-4 | 0.04 | 15 | 8.2 | 5.4 | 21 |
| (Human melanoma, | | | | | |
| B-Raf V599D) | | | | | |
| COLO829 | 0.089 | 3.7 | 7.1 | 6.0 | — |
| (Human melanoma, | | | | | |
| B-Raf V599E) | | | | | |
| A375 | 0.18 | >50 | 5.4 | 4.3 | 43 |
| (Human melanoma, | | | | | |
| B-Raf V599E) | | | | | |

The data in Table 1 show that cancer cell lines having mutated B-Raf are especially sensitive to resorcylic acid lactones having an enone structure amenable to Michael adduct formation, as illustrated by hypothemycin. In contrast, the A549 cell line, having wild-type B-Raf, is less sensitive, although its growth is still significantly inhibited. PD 98059, a MEK inhibitor based on a benzopyran-4-one scaffold, and 5,6-dihydrohypothemycin, having the enone carbon-carbon double bond hydrogenated and thus being unable to participate in Michael reactions, are both poorly effective as inhibitors. Moreover, the enone resorcylic acid lactones are significantly more active against cells with the B-Raf mutation than Bayer 43-9006 (Sorafenib), which was initially developed as a Raf-1 inhibitor and is currently in human clinical trials against melanoma. Likewise, the enone resorcylic acid lactone is much more potent than SU11248, another kinase inhibitor that has been investigated in clinical trials.

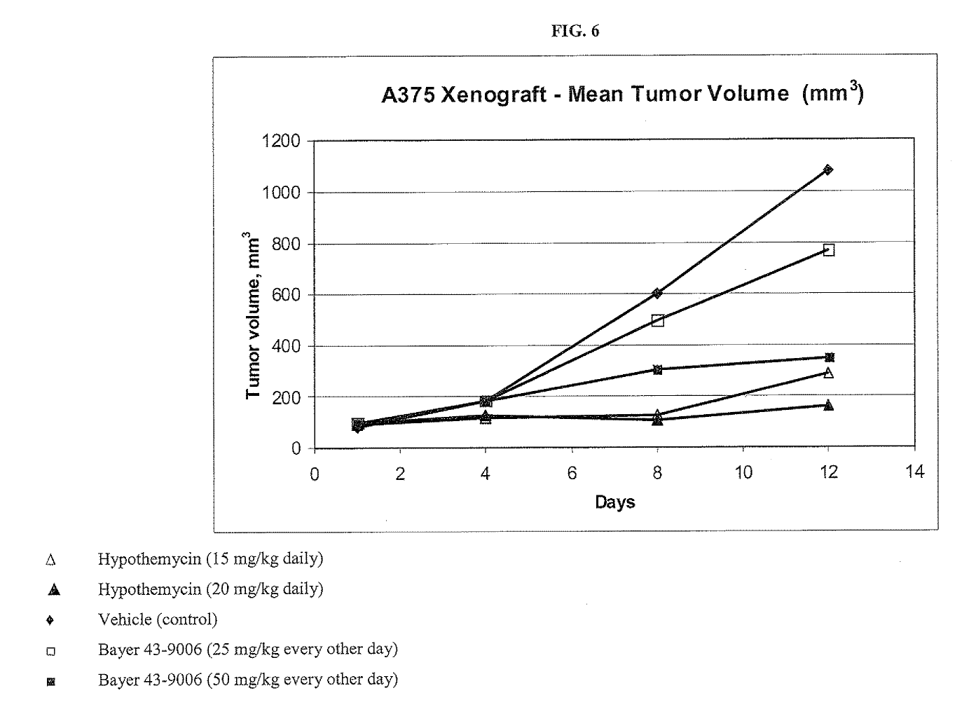

The sensitivity of B-Raf mutated cancer cell lines to RALs was confirmed in a B-Raf mutant melanoma (A375) xenograft model. As seen in FIG. 6, hypothemycin administered daily at 15 mg/kg or 20 mg/kg significantly inhibits the growth of the A375 xenograft relative to vehicle alone. In addition, hypothemycin at both dosages is significantly better than Bayer 43-9006 (a non-RAL, non-cis enone kinase inhibitor) administered at 25 mg/kg or 50 mg/kg every other day, a schedule for Bayer 43-9006 previously reported to be efficacious (Sharma et al., Cancer Res. 2005; 65 (6): 2412-2421). Thus, both in vitro and in vivo analyses demonstrate that cancer cell lines with activating B-Raf mutations are especially sensitive to growth inhibition by RALs.

Use of the compounds of this invention in the treatment of melanoma is of particular interest: ˜70% of malignant melanomas have mutated B-Raf, and melanoma is notoriously difficult to treat once it has progressed beyond the stage where it is treatable by surgical intervention. Likewise, the compounds of this invention are useful in the treatment of colon cancer: ˜20% of colon cancers have mutated B-Raf, and pre-screening biopsy specimens for the BRAF mutation is, in accordance with the methods of the invention, in one embodiment conducted to identify those patients suited for treatment with compounds of this invention. Thus, compounds of this invention are effective in inhibiting the proliferation of cells characterized by mutant B-Raf, in particular V599E (V600E using current nomenclature) and V599D (V600D using current nomenclature) mutations.

### Renal Cell Carcinoma

The methods of the invention include methods for treating renal cell carcinoma (RCC), which accounts for approximately 3% of all adult malignancies, with about 31,000 new cases diagnosed in the United States every year. Cytokine-based immunologic therapy is the current standard of treatment, but only a limited subset of patients responds. Investigation of the biology of RCC has led to the identification of VEGF and its receptors, the VEGFRs (vascular endothelial growth factor receptors) as therapeutic targets (see Rathmell et al., Curr. Opin. Oncol. 2005; 17 (3):261-7). A number of companies, including Onyx and Sugen, are investigating whether VEGFR inhibitors can be used to treat RCC; such compounds are generally inferior to the compounds useful in the present invention, because they only inhibit the receptor, while the compounds of the invention inhibit not only the receptor but also the production of VEGF.

Von Hippel Lindau syndrome is a familial disorder, characterized by mutation of the von Hippel Lindau (VHL) tumor suppressor, which is associated with an increased susceptibility to clear-cell RCC, with a lifetime risk of developing RCC of almost 50%. The VHL protein targets a transcription factor, HIFα, for ubiquitin-dependent proteolysis under normal oxygen conditions. In the absence of functional VHL, HIFα accumulates leading to constitutive expression of the downstream transcriptional targets of HIFα, including VEGF and PDGF. VHL inactivation has also been shown to occur in 60 to 80% of sporadic cases of clear-cell RCC, and VEGF over-expression has been demonstrated in the majority of RCC samples analyzed (Rini et al., J. Clin. Oncol 2005; 23 (5): 1028-43). A monoclonal antibody targeted to VEGF and small molecule VEGFR and PDGFR inhibitors (e.g. Bayer 43-9006) have shown promising results in RCC clinical trials in delaying time to progression or with evidence of either partial response or stable disease in a significant percentage of the patients (see Rini et al., supra). In addition to inhibition of both growth factor receptors VEGFR and PDGFR, the resorcylic acid lactone kinase inhibitors useful in the methods of the present invention also simultaneously target four enzymes of the downstream ERK signaling pathway through inhibition of MEK1/2 and ERK1/2, which has been shown to be constitutively active in RCC (Ahmad et al., Clin. Cancer Res. 2004; 10 (18 Pt 2):6388S-92S, and Oka et al., Cancer Res. 1995; 55 (1.8):4182-7); because VEGF is stimulated by the ERK pathway, the inhibitors useful in the methods of the invention also decrease VEGF production. Hypothemycin and its analogs and derivatives can as provided herein be administered to patients in accordance with the methods of the invention for the treatment of RCC.

### Ras-Dependent Cancers

The methods of the invention include methods for treating Ras dependent cancers. The mitogen activated protein kinase (MAPK) signaling pathway or ERK pathway regulates the growth and survival of cells in many human tumors (Sebolt-Leopold et al., Nat. Rev. Cancer 2004; 4 (12):937-47). Many types of cancer cells exhibit constitutive activation of the MAPK signaling pathway caused by activating mutations in Ras. These mutations lead to increased signaling through the MAPK pathway and increased cell proliferation and include mutations in K-Ras (prevalence of 45% in colon cancer; 90% in pancreatic cancer; and 35% in non-small-cell lung cancer); N-Ras (prevalence of 15% in melanoma, and 30% of ALL and AML); and H-Ras (together with K-Ras and N-Ras mutations, prevalence of 60% in papillary thyroid cancer), Inhibitors of Raf (e.g. BAY 43-9006) or MEK (e.g. PD184352) have been demonstrated to inhibit both growth and the MAPK pathway in human tumor cell lines carrying activating Ras mutations, and in mouse tumor models, have been shown to inhibit tumor growth (Sebolt-Leopold et al., Nat. Med. 1999; 5 (7):810-6, and Sebolt-Leopold, Oncogene 2000; 19 (56):6594-9). Hypothemycin and its derivatives and analogs are potent inhibitors of the MAPK signaling pathway through inhibition at two levels of the cascade, MEK1/2 and ERK1/2, and can be used in accordance with the methods of the invention for the treatment of tumors carrying Ras activating mutations.

### Prostate Cancer

The compounds and methods of the invention are also useful in the treatment of prostate cancer. Prostate cancer is the most prevalent cancer in men with over 1.3 M patients in the US alone. It was projected that, in 2003, there would be 221,000 new cases of prostate cancer, and 29,000 men would die of metastatic prostate cancer despite the use of androgen ablation therapy. Androgen withdrawal is the only effective therapy for patients with advanced disease, and approximately 80% of patients achieve symptomatic and/or objective response after androgen ablation. However, progression to androgen independence ultimately occurs in almost all patients. Although numerous non-hormonal agents have been evaluated in patients with hormone-refractory prostate cancer, these agents have limited antitumor activity with an objective response rate of 20% and no demonstrated survival benefit. Therefore, the identification and selected inhibition of molecular targets that mediate the progression of prostate cancer will have great impact on future treatment of this disease.

An increase in mitogen-activated protein kinase (MAPK) activity has been correlated with the progression of prostate cancer to advanced disease in humans (Gioeli et al., Cancer Res. 1999; 59:279-84). These results, together with observations that Ras activity regulates the androgen requirement of prostate tumor growth in xenografts, indicate that the MAPK pathway plays an important role in prostate cancer proliferation (Bakin et al., Cancer Res. 2003; 63:1981-9; Bakin et al., Cancer Res. 2003; 63:1975-80). The family of serine/threonine protein kinases, the p90 ribosomal S6 kinases (RSK), function as downstream effectors of MAPK. The RSK family consists of four isoforms, which are the products of separate genes. RSKs play an important role in cell survival and proliferation in somatic cells through their ability to phosphorylate and regulate the activity of key substrates, including several transcription factors and kinases, the cyclin-dependent kinase inhibitor, p27Kip1, the tumor suppressor, tuberin, and the proapoptotic protein, Bad. These observations combined with the known importance of MAPK in prostate cancer, indicate that RSKs also contribute to prostate cancer progression.

It has recently been shown (Clark et al., Cancer Res. 2005; 65 (8): 3108-16) that increasing RSK isoform 2 (RSK2) levels in the human prostate cancer line LNCaP enhances prostate-specific antigen (PSA) expression, whereas inhibiting RSK activity using a RSK-inhibitor, 3Ac-SL0101, decreased PSA expression. RSK levels are higher in ˜50% of human prostate cancers compared with normal prostate tissue, indicating that increased RSK levels participate in the rise in PSA expression that occurs in prostate cancer. Furthermore, 3Ac-SL0101 inhibited proliferation of the LNCaP line and the androgen-independent human prostate cancer line PC-3. These results indicate that proliferation of some prostate cancer cells is dependent on RSK activity and that RSK is an important chemotherapeutic target for prostate cancer.

Hypothemycin and its derivatives and analogs potently inhibit two key points of the ERK pathway and the C-terminal kinase domain of the RSK iso forms. Thus, the Michael adduct forming RALs of the invention are useful in accordance with the methods of the invention in the treatment of prostate cancer and metastatic prostate cancer by monotherapy and in combination with androgen ablation therapy.

### Breast Cancer

The methods and compounds of the invention are also useful in the treatment of breast cancer. Breast cancer cases among females in 2003 were estimated to be 210,000 with 40,000 deaths, making this one of the most prevalent forms of cancer. Breast cancer presents as either estrogen receptor-α (ERα) positive or as ERα negative. The presence of ERα is correlated with a better prognosis both in terms of increased disease-free survival and overall survival. ERα-negative breast tumors tend to over-express growth factor receptors such as EGFR and erbB-2 (HER2). Raf-1 is a key intermediate in the signal transduction pathways of these receptors. High levels of constitutive Raf kinase or downstream MAP kinase activity imparts ERα-positive breast cancer cells with the ability to grow in the absence of estrogen, mimicking the ERα-negative phenotype. Abrogation of Raf signaling via treatment with MEK inhibitors can restore the ERα-positive behavior (Oh et al., Mol. Endocrinol. 2001; 15 (8):1344-59). Treatment with antiestrogens, such as tamoxifen, is commonly used to inhibit the growth of ERα-positive cancer cells by inducing cell cycle arrest and apoptosis. This requires the action of the cell cycle inhibitor, p27Kip1. Constitutive activation of the MAPK signaling pathway in ERα-positive cells reduces p27 phosphorylation, and the cdk2 inhibitory activity of the remaining p27, which together contribute to antiestrogen resistance (Donovan et al. J. Biol. Chem. 2001; 276 (44):40888-95). Resistance to cytotoxic drugs like paclitaxel, doxorubicin and 5-fluorouracil is mediated by, in part, Ras-signaling, the upstream effector of Raf. Inhibition of Ras/Raf signaling by treatment with MEK kinase inhibitors counteracts the resistance to a considerable degree (Jin et al., Br. J. Cancer 2003; 89 (1): 185-91). These facts justify the use of signal transduction inhibitors in treatment of breast cancer (Nahta et al., Curr Med Chem Anti-Canc Agents 2003; 3 (3):201-16), which is underscored by the report that the dual use of a MEK and EGFR inhibitor results in significantly more growth inhibition and apoptosis of breast cancer cells than the use of either drug alone (Lev et al., Br. J. Cancer 2004; 91 (4):795-802). Also, EGFR and HER2, proven targets for breast cancer, transmit their proliferative activity through the ERK pathway. Finally, inhibition of the effects of VEGF by the monoclonal antibody Avastin has led to dramatic improvement in the response rate of breast cancer to chemotherapy. Hypothemycin and its analogs and derivatives capable of Michael adduct formation as described herein are potent inhibitors of four enzymes of the ERK pathway, MEK1/2 and ERK1/2, subsequent VEGF production, as well as VEGFR, and can be used in accordance with the methods of the invention to treat breast cancer.

### Pancreatic Cancer

The methods of the invention also include methods for treating pancreatic cancer. Although pancreatic cancer has an incidence of only about 10 cases/100,000 persons, it is the fourth to fifth leading cause of cancer-related deaths in the Western world. Most of the newly diagnosed patients present at an already unresectable tumor stage. The 5-year survival rate of these patients is less than 1%, and the median survival time is approximately 5-6 months after tumor detection. In recent years, increasing attention has been directed towards the role of growth factors in the pathogenesis of human tumors. Human pancreatic cancers over-express a number of important tyrosine kinase growth factor receptors and their ligands, such as those belonging to the epidermal growth factor (EGF), fibroblast growth factor (FGF), insulin-like growth factor (IGF-1), vascular endothelial growth factor (VEGF), and platelet derived growth factor (PDGF) families (Korc, Surg. Oncol Clin. N. Am. 1998; 7 (1):25-41; Ozawa et al., Teratog. Carcinog. Mutagen. 2001; 21(1):27-44; and Ebert et al., Int. J. Cancer 1995; 62 (5):529-35). It is thought that these growth factors act in an autocrine and/or paracrine manner to stimulate pancreatic cancer growth through activation of the ERK pathway. Mutations in the K-Ras oncogene occur with a 75-90% frequency in pancreatic cancer (Li, Cancer J. 2001; 7 (4):259-65), which accentuates the proliferative growth of this cancer. Small molecule inhibitors of receptor tyrosine kinases and downstream signaling kinases (MEK and p38) have been reported to block the proliferation of pancreatic cancer cells in culture (Matsuda et al., Cancer Res. 2002; 62 (19):5611-7, and Ding et al., Biochem. Biophys. Res. Commun. 2001; 282 (2):447-53). Hypothemycin and its analogs and derivatives as described herein are potent inhibitors of PDGFR, VEGFR, MEK, and ERK kinases as well as excessive mitogenic signaling due to mutant K-ras, and can be used in accordance with the methods of the invention in the treatment of pancreatic cancer.

### Epithelial Ovarian Cancer

The compounds and methods of the invention are also useful in the treatment of ovarian cancer. Epithelial ovarian cancer (EOC) is the leading cause of mortality among gynecological malignancies and the fifth leading cause of cancer-related death in women. In 2003, it was predicted that 24,000 new cases would occur with 14,000 deaths. Most patients present with advanced stage ovarian tumors, and treatment is based on extensive surgery followed by chemotherapy. The backbone of chemotherapeutic regimens remains a platinum derivative, to which taxanes have been added in recent years. The MAPK signaling pathway, especially the ERK1/2 serine-threonine kinases, plays a major role in ovarian cancer (Choi et al., Reprod. Biol. Endocrinol 2003; 1 (1):71). This pathway is activated by the platinum-containing or taxane-based chemotherapeutic drugs, such as cis-platin, carba-platin, docetaxel, and paclitaxel, that are commonly used to treat ovarian cancer, and by gonadotropbins and follicle cell stimulating hormone. Drug resistant cells can be restored to drug sensitive cells by treatment with MEK1/2 inhibitors. Thus, in one embodiment, the invention provides a method for treating ovarian cancer, said method comprising administering a protein kinase inhibitor capable of Michael adduct formation with MEK1/2 and ERK1/2 protein kinases in combination with or after administration of a platinum containing anti-cancer drug or a taxane.

Metastasis of ovarian cancer cells can be inhibited by treatment with ERK pathway inhibitors. About 39% of ovarian tumors express PDGFR, and hence an active ERK pathway, and the level of its expression is correlated with higher histological grade and advanced surgical stages of ovarian tumors. Furthermore, stage for stage, patients with PDGFR-A positive tumors had shorter survival times than those with negative tumors. Imatinib (Gleevec) inhibits ovarian cancer cell growth at clinically relevant concentrations through a mechanism that is dependent on inhibition of PDGFR-A (Matei et al., Clin. Cancer Res. 2004; 10 (2):681-90). Peritoneal dissemination is critical for the progression of ovarian cancer. Hepatocyte growth factor induces migration and invasion of ovarian cancer cells by activation of the Ras/Raf/MEK/ERK signaling pathway (Ueoka et al., Br. J. Cancer 2000; 82 (4):891-9), which supports the use of MEK and ERK inhibitors as provided by the present invention to treat this disease. Hypothemycin and its derivatives and analogs are potent covalent inhibitors of PDGFRA, as well as the downstream enzymes PDGFR activates, MEK1/2 and ERK1/2, and can be used in accordance with the methods of the invention in the treatment of ovarian cancer.

### Lung Cancer

The methods of the invention also include methods for treating lung cancer. Lung cancer is the leading cause of cancer mortality in the United States. A 2003 survey predicted the occurrence of 171,000 new cases with 157,000 deaths in that year. In spite of recent advances in therapy, outcomes for locally advanced metastatic cases are still poor. Non-small cell lung cancer (NSCLC) accounts for >75% of all lung cancers in the US. Chemotherapy has an important role for management of advanced stages of the disease. Current drugs include platinum-based combination therapy and docetaxel for second-line treatment. The EGFR is expressed or over-expressed in most epithelial tumors including lung; NSCLC squamous-cell carcinomas show an 80% over-expression.

While gefitinib (Iressa) has been approved in the US for treatment of NSCLC in patients that failed other chemotherapies, the involvement of the MAPK pathways in EGFR derived signaling demonstrates that other targets are available for treatment of this troubling cancer. VEGFR-2 (KDR) and VEGFR-3 (Flt-4) are expressed in NSCLC (Tanno et al., Lung Cancer 2004; 46 (1): 11-9), and increased amounts of their ligands or hypoxic conditions stimulated the proliferation and migration of cultured NSCLC cancer cell types. Stimulation of KDR and Flt-4 also resulted in enhanced activity of the MAPK pathway. Similarly, 34% of the tissue samples from patients with NSCLC showed hyper-activation of the ERK pathway (Vicent et al., Br. J. Cancer 2004; 90 (5): 1047-52). A strong correlation between the phosphorylation status of ERK2 and Akt, two of the signaling kinases controlled by the EGFR, and gefitinib therapy has also been described (Cappuzzo et al., J. Natl. Cancer Inst. 2004; 96 (15): 1133-41).

These and other recent clinical observations (Cesario et al., Curr. Med. Chem. Anti-Canc. Agents 2004; 4 (3):231-45) justify the expanded use of inhibitors of signaling protein kinases in the treatment of NSCLC, including combination therapy with topoisomerase inhibitors (Maulik et al., J. Environ. Pathol. Toxicol Oncol. 2004; 23 (4):237-51) and other types of established cancer drugs. Finally, inhibition of the effects of VEGF by the monoclonal antibody Avastin has led to dramatic improvement in the response rate of NSCLC cancer to chemotherapy with paclitaxel and carboplatin. Hypothemycin and its derivatives and analogs as provided herein are potent inhibitors of the receptor kinases KDR (VEGFR), Flt-4, and cKIT shown to be important in lung cancer, as well as four enzymes of the ERK pathway, MEK1/2 and EKR1/2, which regulate subsequent VEGF production, and can be used in accordance with the methods of the invention to treat lung cancer in mono- and combination therapy.

### Colorectal Cancer

Colorectal cancer is the second leading cause of cancer deaths in the United States and accounts for about 15% of human malignancies. The American Cancer Society estimated nearly 150,000 new cases of colorectal cancer would be diagnosed in the year 2003 (Jemal et al., CA Cancer J Clin 2003, 53:5-26). The majority of patients with advanced colorectal cancer ultimately experience a recurrence of their cancer that is considered incurable. Standard treatment involves surgical resection and sometimes radiation treatment, whereas chemotherapy, for example, with the standard Camptosar® (irinotecan HCl injection)/5fluorouracil/leucovorin regimen, is far from being satisfactory.

Epidemiological and gene mapping studies have shown that many types of colon cancer involve aberrations in cell signaling pathways. For instance, in the MAPK pathway, B-Raf V599E (V600E) mutants are found in ˜15% of colon cancers and lead to constitutive activation of the ERK pathway necessary for cell proliferation (Sebolt-Leopold et al., Nat Rev Cancer 2004, 4:937-47). Specific inhibitors of MAPK signaling are therefore effective in inhibiting the proliferation of cells with the Raf V599E (V600E) mutation (Sebolt-Leopold et al., supra; ibid. Nat Med 1999, 5:810-6). As described in Example 5 below, the ERK pathway in the B-Raf V599E (V600E) cell line COLO829 is completely shut down after a 10 min. exposure to the MEK1/2 and ERK1/2 inhibitor hypothemycin at sub-micromolar concentrations. Similar results are seen in the B-Raf V599E mutant colon cancer cell line HT29. Less effective MEK1/2 inhibitors like CI-1040, PD0325901 and ARRY-142886 are effective in animal models of colon cancer (Sebolt-Leopold et al., supra).

Colon cancer metastasis involves secretion of matrix metalloproteases (MMP); a MEK1/2 inhibitor can block MMP-7 gene expression in colon cancer cells (Lynch et al., Int J Oncol 2004, 24:1565-72); ERK1/2 inhibitors also have this property, because ERK2 is involved in integrin alpha(v)beta6 mediated MMP-9 expression by colon cancer cells (Gu et al., Br J Cancer 2002, 87:348-51). Specific inhibitors of the ERK and/or p38 dependent MAPK signaling pathways are also useful, in accordance with the methods of the invention, for treatment of colon cancer in other contexts: potentiation of the ability of non-steroidal anti-inflammatory drugs to stimulate apoptosis of colon cancer cells (Nishihara et al., J Biol Chem 2004, 279:26176-83; Sun and Sinicrope, Mol Cancer Ther 2005, 4:51-9), inhibition of the ability of gastrin-17 to promote colon cancer growth by stimulation of CCK-2 receptor mediated prostaglandin E2 production (Colucci et al., Br J Pharmacol 2005, 144:338-48), and inhibition of the TNF receptor associated factor (TRAF1) induction that is an aspect of tumor promotion in colon cancer via the NFkB pathway (Wang et al., Oncogene 2004, 23:1885-95).

Stimulation of the VEGF receptor can enhance angiogenesis. Monoclonal antibodies like Avastatin (bevacizumab) that bind to VEGF and inhibit the action of VEGF released from cells, were highly successful and approved in 2004 for the treatment of metastatic colon cancer. Results from recent clinical trials indicate that the addition of Avastin to the common chemotherapy regimen 5-fluorouracil/leucovorin as initial therapy improves progression-free survival in advanced colorectal cancer (http://patient.cancerconsultants.com/colon_cancer_news.aspx?id=17462). Previous clinical trials demonstrated an advantage with the addition of Avastin to the chemotherapy regimen Camptosar®/5fluorouracil/leucovorin in the treatment of this disease. It has been shown that neuropilin-1 is a VEGF co-receptor in human colon cancer cells whose formation, and thus ability to stimulate angiogenesis and cell growth, also can be inhibited by ERK1/2 and p38 inhibitors (Parikh et al., Am J Pathol 2004, 164:2139-51).

Resorcyclic acid lactones useful in the methods of the invention are particularly useful in treating colon cancers with the BRAF V599E mutation as well as those that do not have the mutation. In addition to the two-point inhibition of the ERK pathway at MEK1/2 and ERK1/2 present in all cells, they inhibit VEGF production (through inhibition of the ERK pathway) as well as VEGFR, and inhibit TAK1 to inhibit the NFkB pathway.

Basal Cell Carcinoma and Other Cancers Associated with Sonic Hedgehog Pathway

The methods of the invention also include methods for treating basal cell carcinoma and other cancers associated with an activated hedgehog (Hh) pathway. The Hh-signaling pathway comprises three main components: 1) the Hh ligand; 2) a transmembrane receptor circuit composed of the negative regulator Patched (Ptch) plus an activator, Smoothened (Smo); and 3) finally a cytoplasmic complex that regulates the Cubitus interruptus (Ci) or Gli family of transcriptional effectors (see Frank-Kamenetsky et al., Journal of Biology 2002, 1:10). There is positive and negative feedback at the transcriptional level as the Gli1 and Ptch1 genes are direct transcriptional targets of activation of the pathway. The Hh ligands are synthesized as ˜45 kDa precursors that undergo autoprocessing to result in the covalent attachment of a cholesterol moiety to the amino-terminal half of the precursor. Smo is a seven-pass transmembrane protein with homology to G-protein-coupled receptors (GPCRs), while Ptch1 is a twelve-pass transmembrane protein that resembles a channel or transporter. Consistent with its role as an essential pathway inhibitor, removal of Ptch1 results in a constitutively active Hh pathway that functions independently of the Hh ligand. Similarly, specific point mutations in the transmembrane helices of Smo are capable of constitutively stimulating the pathway, effectively bypassing Ptch1 inhibition.

While vital to the proper development of animals, inappropriate hedgehog pathway signaling through mutations or other events that inactivate Ptch1 or activate Smo result in several types of tumors, including basal cell carcinoma, medulloblastomas, rhabdomyosarcomas, gliomas, superficial bladder cancer, gastrointestinal tract tumors, small cell lung cancer (SCLC), pancreatic carcinomas and prostate cancer (di Magliano and Hebrok, Nat Rev Cancer 2003, 3:901-11; Ruiz et al., Nat Rev Cancer 2002, 2:361-72; Fan, et al., Nat Rev Cancer 2002, 2:361-72; Fan et. al., Endocrinology 2004, 145:3961-70; Sanchez et al., Proc Natl Acad Sci USA 2004, 101:12561-6). Hence, inhibitors of Hh signalling can provide valuable leads for drug development of anticancer agents (Romer et al., Cancer Res 2005, 65:4975-8; Taipale et al., Nature 2000, 406:1005-9; Williams, Drug News Perspect 2003, 16:657-62).

Using the Hh-responsive cell line C3H10T1/2, it has been shown that GUI induces the Serum-Response-Element and activates PDGFR, which in turn activates the Ras-ERK pathway and stimulates cell proliferation (Xie et al., Proc. Natl. Acad Sci USA, 2001, 98:9255-9289). Thus, inhibition of PDGFR or the ERK pathway provides blockage of the effects of Hh pathway activation, and would effect the Hh pathway endpoint regardless of the mechanism of Hh activation (i.e. stimulation or release of inhibition).

Basal cell carcinoma (BCC) is the most common human cancer, with over 750,000 new cases per year in the United States. It has been established that mutations of the patched gene (Ptch1 or 2) are associated with the heritable disorder basal cell nevus syndrome as well as sporadic BCCs. The downstream molecule Gli1 mediates the biological effect of the pathway, and it is up-regulated in about 90% of BCCs. Gli1 in turn up-regulates PDGFRα, which causes activation of the ERK pathway that induces cell proliferation. Overproduction of PDGFRα with subsequent activation of the ERK pathway is an important mechanism by which mutations in the hedgehog pathway cause BCC (Xie et al., Proc. Natl. Acad. Sci. USA 2001, 98:9255-9).

Intratumoral IFNα is an effective but inconvenient treatment for BCC, with a remission rate of ˜50 to 80%. Imiquimod, which stimulates secretion of cytokines such as IFNα, is also effective. Recently, it has been shown that IFNα mediated killing in hedgehog pathway-activated BCC cells results from its interference with the ERK pathway, which results in elevated Fas expression and subsequent apoptosis (Li et al., Oncogene, 2004; 23, 1608-17).

The above discussion shows that inhibition of PDGFR or the ERK pathway provides blockage of the effects of Hh pathway activation, and would effect the Hh pathway endpoint regardless of the mechanism of Hh activation. Hypothemycin and its derivatives and analogs as described herein are potent inhibitors of both PDGFRα and two enzymes in the ERK pathway. As shown in Table 4 infra, they are potent inhibitors of BCC cells in culture, and can be used in accordance with the methods of the invention in the treatment of BCC and other tumors caused by an activated hedgehog pathway. Thus, hypothemycin has an IC₅₀ of about 100 nM against the BCC cell line ASZ001 in culture (Table 4). By comparison, Tazarotene, a topical acetylenic retinoid that causes >85% inhibition of development of BCCs in Ptc+/− mice (So et al., Cancer Res. 2004; 64, 4385-9) and is used clinically to treat BCC, inhibits ASZ001 BCC cells with an IC₅₀ of ˜10,000 nM.

### Restenosis

The compounds and methods of the invention are also useful in angioplasty and the use of stents, in that they can prevent restenosis. Smooth muscle cell proliferation is a key event in neointimal formation after angioplasty. PDGF is a mitogenic factor involved in the response of the vascular smooth muscle cells to injury and activates the ERK pathway in smooth muscle cells, which is crucial to migration. MEK inhibitors are effective pharmacological agents for thwarting the proliferation and migration of vascular smooth muscle cells, because they block ERK activation and thereby the cellular response to PDGF. The stress activated MAPK p38 can also be involved in the response to vascular injury, and inhibitors targeted at p38 and upstream kinases that regulate its activity are effective in the treatment of restenosis. The PDGF receptors stimulate smooth muscle migration and proliferation, and the VEGF receptors stimulate neo-angiogenesis. As the compounds useful in the methods of the invention inhibit PDGFR and VEGFR as well as multiple kinases in the ERK and JNK pathways, they are potent inhibitors of restenosis and so are generally useful in the preparation of stents, both cardiac and peripheral, and other devices that stimulate deleterious smooth muscle cell migration.

Thus, in one embodiment, the present invention provides a stent or other device intended for in vivo use coated, embedded with, or otherwise comprising a compound useful in the methods of the present invention that prevents or retards unwanted smooth muscle cell proliferation and migration to the stent. The uncontrolled migration of smooth muscle cells to these stents creates a disease condition treatable in accordance with the methods of the invention. Thus, the stents provided by the present invention represent a significant advance over current stent technology, because they contain potent and irreversible inhibitors of multiple receptors and cell signaling pathways critical for restenosis. In one embodiment, the RAL used to prepare the stent is an RAL useful in the methods of the invention other than hypothemycin or an RAL disclosed in Tremble, US 2004/0243224 A1 (2004).

### Rheumatoid Arthritis

Rheumatoid arthritis (RA) is a connective tissue disease that affects more than 1,000,000 people in the US. This autoimmune disorder is driven largely by the recruitment of activated immune cells (T and B cells) and macrophages to the afflicted joints. There, the cytokines IL-1 and TNF-α produced by these cells mediate the irreversible joint destruction seen in RA. The downstream genes activated by these cytokines, via the NFkB and AP-1 transcription factors induced by the NFkB and MAPK signaling pathways, encode both inflammatory molecules and secreted proteinases of the matrix metalloproteinase (MMP) family, which are found at elevated levels in RA. Compounds that can inhibit cytokine-induced MMP gene expression and also block the NFkB and MAPK signaling pathways can provide new arthritis drugs (Vincenti and Brinckerhoff, J. Clin. Invest. 2001, 108:181. IL-1 induces activation of the MEKKK TAK1. TAK1 controls the activation of NFkB and, through JNK, AP-1 (Ninomiya-Tsuji et al., Nature 1999, 398:252; thus, a specific TAK1 inhibitor can prevent inflammation by blocking the IL-1 induced activation of the NFkB, p38 and JNK pathways. Indeed, specific inhibitors of JNK and of the p38 isoform that predominates in inflamed cells, including RA cells, effectively block expression of genes controlled by JNK and p38 pathways in cultured cells and show considerable reduction in collagenase gene expression and joint destruction in animals. MEK1/2 inhibitors also effectively block IL-1 stimulated responses in cultured cells (Barchowsky et al., Cytokine 2000, 12:1469.

In one embodiment, the present invention provides methods for treating RA with inhibitors capable of forming a Michael adduct with TAK1 and MEK3/6 to inhibit the p38 pathway, TAK1 and MEK4/7 to inhibit the JNK pathway, and MEK1/2 and ERK1/2 to inhibit the ERK pathway; through this extensive sequential and network inhibition, NFkB and AP-1 dependent signaling pathways are effectively inhibited and the disease is treated.

### Psoriasis

The treatment of psoriasis with the compounds useful in the methods of the present invention illustrates the power of the sequential and multiple signaling pathway inhibition approach. Over 10 million people suffer from psoriasis worldwide, and although many treatments exist, few are effective over the long-term, and no cure has been developed (Geilen and Orfanos Clin Exp Rheumatol. 2002; 20 (6 Suppl 28): S81-7; Gniadecki et al., Acta Derm Venereol. 2002; 82 (6): 401-10).

Psoriasis is an inherited spectrum of skin diseases characterized by epidermal hyperproliferation, disturbed differentiation, inflammation and excessive dermal angiogenesis. The pathogenesis of psoriasis is based on immunological mechanisms, defective growth control mechanisms, or on a combination of these mechanisms. Epidermal hyperproliferation, abnormal keratinization, angiogenisis and inflammation are well-established hallmarks of the psoriatic plaque, which generally occur on the joints, limbs and scalp, but which can appear anywhere on the body.

Immunosuppressive and anti-inflammatory drugs are often used to treat psoriasis on the basis of the involvement of T cells in the autoimmune response believed to be important in its etiology (Bowcock et al., Hum Mol. Genet. 2001; 10 (17): 1793-805) either by direct effects or indirectly through the release of various chemokines and cytokines, including TNFα, that signal the keratinocytes to hyperproliferate via activation of the Erk pathway. Integrins and other adhesion molecules are also involved; studies with transgenic mice have shown that integrin over-expression activates the MAPK signaling pathway (ERK pathway), causing an increased growth rate of keratinocytes and re-creating the histological features of psoriasis. Furthermore, constitutive activation of MEK1, especially in the presence of elevated IL-1alpha levels, is sufficient to generate hyperproliferative and inflammatory skin lesions with many of the hallmarks of psoriasis. Recently, the protein kinase STAT3 has been shown to be essential in psoriasis, and inhibition of this enzyme is effective in alleviating the condition (Sano et al., Nat. Med. 2005; 11 (1): 43-49).

Compounds useful in the methods of the present invention for treating psoriasis inhibit a subset of kinases that include MEK1, ERK1/2, VEGFR, PDGFR, MEK4/7 in the JNK (integrin) pathway and TAK1 and MEK3/6 in the p38 stress pathway. As noted above, cell-proliferation in psoriasis is associated with an active ERK pathway, and VEGF is found in high levels in psoriatic skin lesions. Compounds useful in the methods of the invention affect many of the hallmarks of psoriasis: they inhibit cell proliferation through inhibition of the ERK pathway; they inhibit angiogenesis by inhibiting VEGFR; and, through ERK inhibition, production of VEGF and STAT3. Although they do not directly inhibit EGFR, they inhibit the ERK pathway that serves as the link between EGFR and cell proliferation, and they provide dual inhibition (TAK1 and MEK3/6) of the p38 stress pathway. Finally, the integration of three signal pathways leads to the secretion of cytokines and acquisition of the following effector functions by T-cells: (i) the activation of calcineurin, (ii) the activation of the ERK pathway and (iii) the activation of the JNK pathway. Compounds useful in the methods of the invention inhibit MEK and ERK, as well as the JNK pathway, and thus two of the three pathways involved in T-cell activation. Thus, the RALs of the present invention inhibit targets in each of the pathways responsible for the biological hallmarks of psoriasis, and the methods of the invention for treating psoriasis offer substantial promise in the treatment of this disease.

### Inflammatory Bowel Disease

The methods of the invention also include methods for treating inflammatory bowel disease (IBD), including Crohn's disease and ulcerative colitis, by administering therapeutically effective doses of the Michael adduct forming protein kinase inhibitors described herein. These are disorders of unknown aetiology characterized by chronic relapsing inflammation of the gastrointestinal tract leading to abdominal pain and chronic diarrhea. They are multi-factorial diseases caused by the interplay of genetic, environmental and immunological factors. Several treatment options for IBD, in particular Crohn's disease, have been developed based on the inhibition of specific signal transduction elements.

For example, specific inhibition of the central pro-inflammatory cytokine, tumor necrosis factor-α (TNF-α), by the monoclonal anti-TNF-α antibody infliximab has become a mainstay of the treatment of steroid-refractory Crohn's disease. Owing to their importance in inflammatory signal transduction, MAPK pathways are targets for inhibition in acute and chronic inflammation. Multiple MAPK pathways orchestrate the inflammatory responses that are associated with the etiology of IBD. The ERK1/2, p38, JNK/SAPK protein kinases and their associated signaling pathways, for instance, are all involved and are known to be significantly activated in Crohn's disease. Treatment with inhibitors of proteins in these pathways or the upstream kinases that regulate their activity is effective in the clinical treatment of IBD. In one embodiment, the present invention provides methods for treating inflammation and inflammatory diseases, including IBD, with a resorcylic acid lactone that is capable of forming a Michael adduct with multiple enzymes in these pathways. The present invention provides methods for treating these diseases in which potent inhibitors of two sites in the ERK pathway (MEK1/2 and ERK1/2), one in the JNK/SAPK pathway (MEK4/7) and two in the p38 pathway (TAK1 and MEK3/6), are administered to a patient in need of treatment.

### Mastocytosis

The methods of the invention also include methods for treating mastocytosis, a proliferative disorder associated with an excess of mast cells. The two main forms are cutaneous, in which mast cells accumulate in the skin, and systemic, in which mast cells can accumulate in many different tissues (www.niaid.nih.gov/factsheets/masto.htm). Both of these forms may progress to a more aggressive form of the disease, malignant mastocytosis, which, in turn can progress to a form of leukemia (Longley, Cutis 1999; 64 (4):281-2, and Longley et al., Nat. Genet. 1996; 12 (3):312-4). Current therapies for mastocytosis are focused on the relief of symptoms, and no cure for the condition is currently available.

The cKIT protein is a mast cell transmembrane receptor tyrosine kinase that is activated in the presence of mast cell growth factor and stimulates the proliferation of mast cells via activation of the ERK pathway. Mutations of c-KIT, usually D816V, resulting in expression of a constitutively active cKIT, have been observed in both systemic and cutaneous mastocytosis (Longley et al., Proc, Natl. Acad. Set USA 1999; 96 (4): 1609-14). This form of the disease is resistant to imatinib (Gleevec; Ma et al., J. Invest. Dermatol. 1999; 112 (2): 165-70), the first kinase inhibitor drug approved for use in human medicine. Hypothemycin and its derivatives and analogs as described herein are potent inhibitors of wild type KIT and constitutively active KIT (D816V) as well as two points (MEK1/2 and ERK1/2) in the ERK pathway and can be administered to patients in accordance with the methods of the invention as a therapy for mastocytosis. In vitro testing shows that mastocytoma cell lines are sensitive to hypothemycin. With the mouse mastocytoma cell line, P815, that expresses a constitutively active cKIT (D814Y, which corresponds to the D816V mutation in humans), hypothemycin has a GI₅₀ of 310 nM, whereas the other known cKIT inhibitors BAY 43-9006 and SU11248 have GI₅₀S of 310 nM and 320 nM, respectively.

Inflammatory Disease with Mast Cell Component

Compounds useful in the methods of the invention can also be administered to treat inflammatory diseases associated with mast cells. Mast cells are also involved, in the development of other diseases and conditions amenable to treatment in accordance with the methods and compounds of the invention. Mast cells are necessary for the development of allergic reactions through crosslinking of their surface receptors for IgE (FcqRI), leading to degranulation and the release of vasoactive, pro-inflammatory and nociceptive mediators. A main aspect of mast cell physiology, largely ignored until recently, is that mast cells can secrete mediators without overt degranulation, through differential or selective release. This process is believed to be regulated by the action of distinct protein kinases (Theoharides et al., J. Neuroimmunol. 2004; 146 (1-2):1-12).

Unlike allergic reactions, mast cells are rarely seen to degranulate during autoimmune or inflammatory processes. Instead, mast cells appear to undergo ultra-structural alterations of their electron dense granular core indicative of secretion, but without overt degranulation, a process that has been termed “activation”, “intragranular activation”, or “piecemeal” degranulation. Mast cells are involved in inflammatory diseases that include asthma, atopic dermatitis, cardiovascular disease, chronic prostatitis, fibromyalgia, irritable bowel syndrome, interstitial cystitis, migraines, multiple sclerosis (MS), neurofibromatosis, osteoarthritis, rheumatoid arthritis, and scleroderma (Theoharides et al., supra). In fact, many of these diseases appear to occur concomitantly, as in interstitial cystitis. Mast cells are required for autoimmune arthritis, play a vital role in skin hypersensitivity reactions, and are strongly implicated in cardiovascular pathology, especially unstable angina and silent myocardial ischemia. Moreover, their close physical association with nerve endings implicates mast cells in the etiology of many stress induced inflammatory diseases.

The receptor tyrosine kinase, c-Kit (CD117), is essential for mast cell survival (Tsujimura, Pathol. Int. 1996; 46 (12):933-8). The c-Kit ligand, stem cell factor (SCF), is important for human mast cell proliferation and maturation, and withdrawal leads to mast cell apoptosis. Constitutive expression of c-Kit occurs in mast cell disease (Mol et al., J. Biol. Chem. 2003; 278 (34):31461-4). Hypothemycin and its derivatives and analogs as described herein are potent irreversible inhibitors of c-Kit as well as two downstream points (MEK1/2,ERK1/2) of the c-Kit-activated ERK pathway, and the present invention provides methods for treating inflammatory diseases that are influenced or caused by mast cells, including the diseases specifically enumerated above, by administering therapeutically effective doses of an RAL capable of Michael adduct formation with a susceptible protease.

### Pulmonary Fibrosis

The invention also provides methods for treating pulmonary fibrosis. Idiopathic pulmonary fibrosis (IPF) is an inexorably progressive form of interstitial lung disease with no known etiology. Persons diagnosed with IPF have a median survival of less than 3 years. Current therapy involves treatment with anti-inflammatory steroids and immunosuppressive drugs, but the response rate is very low. Interest in the role of profibrotic cytokines such as TGF-β and PDGF in IPF has focused on the fact that such cytokines cause fibroblast transformation, proliferation and accumulation, leading to production and deposition of extracellular matrix, tissue destruction, and loss of lung function (Lasky et al., Environ. Health Perspect. 2000; 108 Suppl 4:751-62, and Sime et al., Clin. Immunol 2001; 99 (3):308-19). Recent work has shown that imatinib can block the progression of bleomycin-induced pulmonary fibrosis in the mouse by inhibition of PDGFR phosphorylation (Aono et al., Am. J. Respir. Crit. Care Med. 2005) and possibly the c-Abl protein kinase (Daniels et al., J. Clin. Invest. 2004; 114 (9): 1308-16). Hypothemycin and its derivatives and analogs as described herein are potent inhibitors of PDGFR, as well as the ERK pathway that transmits the PDGF signal, and the present invention provides methods for the treatment of pulmonary fibrosis by administering therapeutically effective doses of the RALs that can inhibit such protein kinases through Michael adduct formation.

### Macular Degeneration

The present invention also provides methods for treating age related as well as diabetes related macular degeneration and glaucoma due to the involvement of VEGF (VEGFR is a target of the compounds useful in the methods of the invention) and the ERK pathway in the etiology of such diseases. The compounds useful in these methods of the invention inhibit VEGF-mediated angiogenesis not only by inhibiting production of VEGF via inhibition of multiple kinases in the ERK pathway but also by inhibition of VEGF production via ERK pathway inhibition, as wells as VEGFR in endothelial cells. In one embodiment, a compound useful in the methods of the invention is co-administered with another agent for the treatment of macular degeneration to treat this debilitating condition.

### Allergic Dermatitis

The methods of the invention also include methods for treating allergic dermatitis and other diseases where immunosuppression is desired. As noted above, the integration of three signal pathways leads to the secretion of cytokines and acquisition of effector functions by T-cells: (i) the activation of calcineurin, (ii) the activation of the ERK pathway, and (iii) the activation of the JNK pathway. Hypothemycin inhibits the ERK pathway at two points (MEK1/2 and ERK1/2), as well as the JNK pathway at MEK4/7, and thus two of the three pathways involved in T-cell activation. FK506 is a well known immunosuppressant that inhibits effects of calcineurin, and is used in the treatment of atopic dermatitis. In accordance with the methods of the invention, administration of a compound of the invention as provided herein is used to treat atopic dermatitis. In one embodiment, a compound of the invention is co-administered with a compound or drug that inhibits calcineurin or its effects. Such compounds include but are not limited to FK506 and its numerous derivatives reported in the scientific and patent literature; this treatment results in all three of the signaling pathways that lead to the secretion of cytokines (ERK pathway, calcineurin, INK) being inhibited, and provides an effective treatment for allergic dermatitis and other disorders where immunosuppression is desired.

### Pain

The present invention also provides methods for the treatment of pain. Nine percent of the US population suffers from moderate to severe non-cancer-related pain of all types, which includes >15 million individuals with chronic pain. Approximately 26 million patients worldwide (10 million in the US) suffer from some form of neuropathic pain, a type of chronic pain in which the pain is inappropriate to the stimulus. Peripheral neuropathic pain typically develops when peripheral nerves are damaged, as through surgery, bone compression (in various diseases), diabetes, and infection. Two common and severely debilitating symptoms of neuropathic pain conditions are hyperalgesia and allodynia. Hyperalgesia is a heightened pain response generated by a painful stimulus; allodynia is pain from stimuli that are not normally painful. Both are often resistant to conventional analgesics. The general failure of analgesics to treat these conditions may be a consequence of long-term changes in neuronal processing in the spinal cord. Indeed, changes in expression of a variety of neurotransmitters, their receptors and other genes in both the spinal cord and the dorsal root ganglia have been shown to be associated with hyperalgesia (cf. Woolf and Costigan, Proc Natl Acad Sci USA, 1999 Jul. 6; 96 (14):7723-30).

Due to the high incidence and the poor efficacy of current treatments for neuropathic pain, novel targets for this condition are being keenly sought. Protein kinases play important roles in various types of pain. The study of changes in gene expression in drug induced neuropathic pain has identified several key components of the extracellular signal-regulated kinase (ERK) cascade to be altered in both streptozoocin induced diabetic neuropathy and chronic constriction injury animal models of pain (cf. Ciruela et al., 2003 Br J Pharmacol 138 (5): 751-6). Increased levels of ERK1/2 activity in the spinal cord correlated with the onset of hyperalgesia. Intrathecal administration of the MEK1/2 inhibitor PD198306 dose-dependently blocked static allodynia, a common experimental measurement of the pain response, in both models of neuropathic pain. Intraplantar administration of PD198306 had no effect in either model of hyperalgesia. Therefore, the relevant changes in the activation of ERK1/2, which is the main consequence of the effect of MEK1/2 inhibition, must localize to the central nervous system. Other studies have demonstrated the involvement of activated ERK1/2 kinases in dorsal horn neurons of the spinal cord as a consequence either of inflammatory pain hypersensitivity (Ji et al., 2002 J Neurosci 22 (2): 478-85) or of the action of metabotropic glutamate receptor agonists in the spinal cord (Adwanikar et al., 2004 Pain 111 (1-2): 125-35). In each case, a MEK inhibitor was able to ameliorate the pain response. When phosphorylated ERK enters the nucleus, it activates the RSK2 type of kinase, which then activates CREB leading to the cAMP mediated transcription of various genes involved, in the onset of pain responses (Ji et al., 2002 J Neurosci 22 (2): 478-85). Other MAPK signaling pathways have also been implicated in neuropathic pain; for instance, the p38 stress-activated MAPK is activated within one day following ligation of the L5 spinal nerve in adult rats, and the effect persists for >3 weeks (Jin et al., 2003 J Neurosci 23 (10): 4017-22). Intrathecal injection of the p38 inhibitor SB203580 reduced the pain response considerably, especially when given at early time points following induction of neuropathy.

Each of the resorcylic acid lactone inhibitors described herein can inhibit multiple protein kinases associated with pain, and is thus a valuable analgesic agent. Each is a potent inhibitor of the central portion of the MEK/ERK signaling pathway at two points, inhibiting some four enzymes (MEK1/2 and ERK1/2); each inhibits the p38 pathway by inhibiting TAK1 and MEK3/6. In addition, to inhibiting two points of the ERK pathway, each inhibits the downstream RSK2 type of kinase thus blocking multiple steps in the path leading to CREB activation. The present invention accordingly provides methods for treating pain that comprise the administration of therapeutically effective doses of an RAL inhibitor that can form a Michael adduct with the susceptible protein.

### Combination Therapies

Certain anti-cancer compounds are known to activate the ERK pathway in certain cell types, and so are, in one aspect of the methods of the invention, co-administered with an RAL useful in the methods of the invention. Taxol and other tubulin interacting agents can induce activation of the ERK pathway in cancer cells (Stone and Chambers (2000) Exp Cell Res 254:110-119; MacKeigan et al. (2000) J Biol Chem 275:38953-38956; McDaid and Horwitz (2001) Mol Pharmacol, 60:290-301). This occurs in some cells, such as HeLa and CHO cells, but not in others such as MCF-7 cells (McDaid and Horwitz (2001), supra). Further, when cells exhibiting paclitaxel-induced ERK activation are treated with the MEK inhibitor U0126, additivity of apoptosis and cytotoxicity is observed. Similarly, the ERK pathway is activated by carboplatin and cis-platin (Choi et al., Reprod. Biol. Endocrinol. 2003; 1 (1):71). It is believed that certain cancer cells activate the ERK pathway in an accommodative response to the stress of certain agents that, in effect, results in a resistance mechanism. In such cases, drug resistant cells can be converted to drug sensitive cells by treatment with ERK pathway inhibitors (Choi et al., Reprod. Biol. Endocrinol. 2003; 1 (1):71). Accordingly, in one embodiment, the methods of the invention for treating cancer or a particular cancer indication, comprise the administration of an anti-cancer compound that activates the ERK pathway, including but not limited to a taxane such as docetaxel or paclitaxel or other microtubule stabilizing or destabilizing agent, including but not limited to an epothilone, such as epothilone B or D or an epothilone derivative, or a platinum agent, such as cisplatin or carboplatin, in combination with a RAL as described herein to the patient to treat the ERK pathway-dependent cancer.

In another combination therapy of the invention, a RAL protein kinase inhibitor capable of forming a Michael adduct with a kinase that is itself, or is activated by, a client protein of Hsp90 is co-administered with an Hsp90 inhibitor. Here, the RAL enone inhibits its specific kinases, and the Hsp90 inhibitor results in destruction of the same or different set of kinases that serve as Hsp90 client proteins. In one embodiment, the HSP90 inhibitor is geldanamycin or a geldanamycin analog such as 17-AAG or 17-DMAG. In another combination therapy of the invention a RAL protein kinase inhibitor capable of forming a Michael adduct with its target protein kinase is co-administered with a topoisomerase inhibitor.

Thus, when used for the treatment of human disease, the compounds useful in the methods of the invention can be administered in combination with other pharmaceutical agents. For example, the expected MAPK pathway inhibitors typically exert a cytostatic effect on cells in which the ERK, JNK or other MAPK pathway is activated by mitogens, aberrantly functional mitogenic receptors (e.g., VEGFR or PDGFR), mutant Ras or Raf proteins, aberrantly activated MEKK enzymes, or constitutively expressed ERK genes. In contrast, the commonly used cancer chemotherapy drags typically exert a cytotoxic effect. Thus, the MAPK pathway inhibitors of the invention can be administered in combination chemotherapy with established cytotoxic drugs, or newer drugs like the Hsp90 inhibitory geldanamycin analogs 17-AAG and 17-DMAG, whose antitumor effects complement those of MAPK pathway inhibitors.

Anti-cancer or cytotoxic agents that can be co-administered with compounds useful in accordance with the methods of the invention include alkylating agents, angiogenesis inhibitors, antimetabolites, DNA cleavers, DNA crosslinkers, DNA intercalators, DNA minor groove binders, enediynes, heat shock protein 90 inhibitors, histone deacetylase inhibitors, microtubule stabilizers, nucleoside (purine or pyrimidine) analogs, nuclear export inhibitors, proteasome inhibitors, topoisomerase (I or II) inhibitors, tyrosine kinase inhibitors. Specific anti-cancer or cytotoxic agents include β-lapachone, ansamitocin P3, auristatin, bicalutamide, bleomycin, bortezomib, busulfan, callistatin A, camptothecin, capecitabine, CC-1065, cisplatin, cryptophycins, daunorubicin, disorazole, docetaxel, doxorubicin, duocarmycin, dynemycin A, epothilones, etoposide, floxuridine, floxuridine, fludarabine, fluoruracil, gefitinib, geldanamycin, 17-allylamino-17-demethoxy-geldanamycin (17-AAG), 17-(2-dimethylaminoethyl)amino17-demethoxygeldanamycin (17-DMAG), gemcitabine, hydroxyurea, imatinib, interferons, interleukins, irinotecan, maytansine, methotrexate, mitomycin C, oxaliplatin, paclitaxel, suberoylanilide hydroxamic acid (SAHA), thiotepa, topotecan, trichostatin A, vinblastine, vincristine, and vindesine.

### Treatment of Cancers Generally

Compounds of this invention can be used for treating diseases such as, but not limited to, hyperproliferative diseases, including: cancers of the head and neck which include tumors of the head, neck, nasal cavity, paranasal sinuses, nasopharynx, oral cavity, oropharynx, larynx, hypopharynx, salivary glands, and paragangliomas; cancers of the liver and biliary tree, particularly hepatocellular carcinoma; intestinal cancers, particularly colorectal cancer; treat ovarian cancer; small cell and non-small cell lung cancer; breast cancer sarcomas, such as fibrosarcoma, malignant fibrous histiocytoma, embryonal rhabdomysocarcoma, leiomysosarcoma, neurofibrosarcoma, osteosarcoma, synovial sarcoma, liposarcoma, and alveolar soft part sarcoma; neoplasms of the central nervous systems, particularly brain cancer; lymphomas such as Hodgkin's lymphoma, lymphoplasmacytoid lymphoma, follicular lymphoma, mucosa-associated lymphoid tissue lymphoma, mantle cell lymphoma, B-lineage large cell lymphoma, Burkitt's lymphoma, and T-cell anaplastic large cell lymphoma. Clinically, practice of the methods and use of compositions described herein will result in a reduction in the size or number of the cancerous growth and/or a reduction in associated symptoms (where applicable). Pathologically, practice of the method and use of compositions described herein will produce a pathologically relevant response, such as: inhibition of cancer cell proliferation, reduction in the size of the cancer or tumor, prevention of further metastasis, and inhibition of tumor angiogenesis. The method of treating such diseases comprises administering a therapeutically effective amount of an RAL as described herein, alone or in combination with another anti-cancer agent, to a subject. The method may be repeated as necessary for therapeutic benefit.

### Non-Cancer Diseases of Cellular Hyperproliferation

The present invention also provides methods for the treatment of non-cancer disorders that are characterized by cellular hyperproliferation by administration to a patient in need of such treatment an RAL compound as described herein. Illustrative examples of such disorders include but are not limited to: atrophic gastritis, inflammatory hemolytic anemia, graft rejection, inflammatory neutropenia, bullous pemphigoid, coeliac disease, demyelinating neuropathies, dermatomyositis, inflammatory bowel disease (ulcerative colitis and Crohn's disease), multiple sclerosis, myocarditis, myositis, nasal polyps, chronic sinusitis, pemphigus vulgaris, primary glomerulonephritis, psoriasis, surgical adhesions, stenosis or restenosis, scleritis, scleroderma, eczema (including atopic dermatitis, irritant dermatitis, allergic dermatitis), periodontal disease (i.e., periodontitis), polycystic kidney disease, and type I diabetes. Other examples include vasculitis (e.g., Giant cell arteritis (temporal arteritis, Takayasu's arteritis), polyarteritis nodosa, allergic angiitis and granulomatosis (Churg-Strauss disease), polyangitis overlap syndrome, hypersensitivity vasculitis (Henoch-Schonlein purpura), serum sickness, drug-induced vasculitis, infectious vasculitis, neoplastic vasculitis, vasculitis associated with connective tissue disorders, vasculitis associated with congenital deficiencies of the complement system, Wegener's granulomatosis, Kawasaki's disease, vasculitis of the central nervous system, Buerger's disease and systemic sclerosis); gastrointestinal tract diseases (e.g., pancreatitis, Crohn's disease, ulcerative colitis, ulcerative proctitis, primary sclerosing cholangitis, benign strictures of any cause including ideopathic (e.g., strictures of bile ducts, esophagus, duodenum, small bowel or colon); respiratory tract diseases (e.g., asthma, hypersensitivity pneumonitis, asbestosis, silicosis and other forms of pneumoconiosis, chronic bronchitis and chronic obstructive airway disease); nasolacrimal duct diseases (e.g., strictures of all causes including idiopathic); and eustachean tube diseases (e.g., strictures of all causes including idiopathic).

### Pharmaceutical Compositions and Dosing

The present invention provides pharmaceutical compositions and preparations comprising a compound useful in a method of the invention. These compositions and preparations include various forms, such as solid, semisolid, and liquid forms. In general, the pharmaceutical preparation contains one or more of the compounds useful in the methods of the invention as an active ingredient and a pharmaceutically acceptable carrier or excipient. Typically the active ingredient is in admixture with an organic or inorganic carrier or excipient suitable for external, enteral, or parenteral application. The active ingredient may be compounded, for example, with the usual non-toxic, pharmaceutically acceptable carriers for tablets, pellets, capsules, suppositories, pessaries, solutions, emulsions, suspensions, and any other form suitable for use. In particular, intravenous and oral modes of administration are contemplated, and the present invention provides pharmaceutical compositions suitable for such modes.

Excipients that may be used include carriers, surface active agents, thickening or emulsifying agents, solid binders, dispersion or suspension aids, solubilizers, colorants, flavoring agents, coatings, disintegrating agents, lubricants, sweeteners, preservatives, iso-tonic agents, and combinations thereof. The selection and use of suitable excipients is taught in Gennaro, ed., Remington: The Science and Practice of Pharmacy, 20th Ed. (Lippincott Williams & Wilkins 2003), the disclosure of which is incorporated herein by reference.

The amount of active ingredient that may be combined with the carrier materials to produce a single dosage form will vary depending upon the host treated and the particular mode of administration. For example, a formulation intended for oral administration to humans may contain carrier material, which may vary from about 5 percent to about 95 percent of the total composition. Dosage unit forms will generally contain from about 5 mg to about 500 mg of active ingredient.

A therapeutically effective amount of compounds of this invention may be administered to a subject in a single or in divided doses. The frequency of administration can be daily, or according some other regular schedule (e.g., every 3rd day), or even according to an irregular schedule. The dosage can be in amounts, for example, of from about 0.01 to about 10 mg/kg body weight, or more usually, from about 0.1 to about 2 mg/kg body weight.

It will be understood, however, that the specific dose level for any particular patient may depend, on a variety of factors. These factors include the activity of the specific compound employed; the age, body weight, general health, sex, and diet of the subject; the time and route of administration and the rate of excretion of the drug; whether a drug combination is employed in the treatment; and the severity of the particular disease or condition for which therapy is sought.

Irreversible inhibitors, such as the compounds discussed herein, have certain distinguishing characteristics that impact the regimen by which they are administered. The target kinases are rapidly inhibited and the inhibitory effect is prolonged, requiring their resynthesis for recovery of the signaling activity. Thus, irreversible inhibitors do not necessarily need to achieve as high plasma concentrations or long plasma half-lives for efficacy, compared to reversible inhibitors. (See, for example, the discussion of CC-1033, an irreversible inhibitor of EGFR function, in Calvo et al., Clin. Cancer Res. 2004, 10:7112-7120). In addition, irreversible inhibitors can be dosed less frequently since their inhibitory effect is longer. The reduction in the exposure required to inhibit growth of a tumor can also reduce toxicity. The unique characteristics of irreversible inhibitors drive optimization of the dosing regimen based on inhibition and recovery of the target kinases in the tumor rather than or in addition to standard pharmacokinetic studies of exposure.

Where applicable, compounds of this invention may be formulated as microcapsules and nanoparticles. General protocols are described for example, in Bosch et al., U.S. Pat. No. 5,510,118 (1996); De Castro, U.S. Pat. No. 5,534,270 (1996); and Bagchi et al., U.S. Pat. No. 5,662,883 (1997), which are all incorporated herein by reference. By increasing the ratio of surface area to volume, these formulations allow for the oral delivery of compounds that would not otherwise be amenable to oral delivery.

As noted hereinabove, compounds of this invention can be co-administered in combination with other pharmaceuticals, in particular other anti-cancer agents. The co-administration may be simultaneous or sequential.

As noted above, the present invention includes within its scope prodrugs of the compounds of this invention, and the present invention provides pharmaceutical compositions comprising such prodrugs. Such prodrugs are in general functional derivatives of the compounds that are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to a subject in need thereof. Conventional procedures for the selection and preparation of suitable prodrug derivatives are described, for example, in Wermuth, “Designing Prodrugs and Bioprecursors,” in Wermuth, ed., The Practice of Medicinal Chemistry, 2nd Ed., pp. 561-586 (Academic Press 2003). Prodrugs include esters that hydrolyze in vivo (for example in the human body) to produce a compound of this invention or a salt thereof. Suitable ester groups include, without limitation, those derived from pharmaceutically acceptable aliphatic carboxylic acids, particularly alkanoic, alkenoic, cycloalkanoic and alkanedioic acids, in which each alkyl or alkenyl moiety preferably has no more than six carbon atoms. Illustrative esters include formates, acetates, propionates, butyrates, acrylates, citrates, succinates, and ethylsuccinates.

### Compounds Useful in the Methods of the Invention

Those of skill in the art will appreciate, in view of the instant disclosure, that there are a large number of resorcylic acid lactones and derivatives that are capable of forming Michael adducts with susceptible protein kinases as described herein. Various RAL compounds have been made and tested, and many more that have been described in the extensive patent literature relating to them. The methods of the present invention arise in part from the discoveries that only a small subset of the existing and imaginable resorcylic acid lactone and derivative class of compounds can be used to achieve inhibition via a slowly reversible Michael addition with a key Cys residue in only a small subset of the kinase family of proteins. These discoveries provide a powerful impetus for re-examining known compounds in pre-clinical testing as agents to treat diseases not previously believed to be amenable to treatment with such compounds, and to make and test compounds that have to date merely been predicted as useful in the patent literature.

Thus, while a previously known and tested compound can be useful in certain methods of the invention, other methods of the invention do not include the use of such compound.

In one embodiment, the compounds and pharmaceutical compositions administered in the therapeutic methods of the invention are compounds described in Eisai Co. Ltd. patent publication Nos. US 2004/0224936 A1 (2004), WO 03/076424 A1 (2003), and WO 2005/023792 A1 (2005), incorporated herein by reference, or compounds that are included within the scope of certain generic compound descriptions in such publications. These publications recite that the compounds described therein may exhibit activity as inhibitors of NF-κB and AP-1 activation and protein kinases (e.g., MEKK, MEK1, VEGFR, PDGFR) but are silent regarding other protein kinases in the kinome that play important roles in particular disease states and conditions. These publications state that the compounds may have application in the treatment of cancer and inflammatory and immune disorders and include descriptions of RA, psoriasis, angiogenesis, and stent technology. However, in view of the limited data available, the therapeutic potential of the compounds disclosed could not be discerned from these publications. Moreover, as disclosed herein, such compounds do not inhibit MEKK1 by Michael adduct formation, which the MEKK1 cannot form with, the compounds of the invention. In addition, as discussed above and described in the Examples below, certain compounds within the scope of the generic compound descriptions of these patent publications do not form the Michael adduct; thus, the compounds useful in the methods of the invention include a novel subset of the compounds generically encompassed by the descriptions of the compounds in these publications.

The present invention teaches that the compounds disclosed in these Eisai patent publications can be used to treat a variety of cancers, including but not limited to AML, basal cell carcinoma, B-Raf mutation-dependent cancers including but not limited to colon cancers and melanoma, breast cancer, GI stromal tumors, Ras dependent cancers, renal cell carcinoma, and prostate cancer, and other conditions, including pulmonary fibrosis, mastocytosis, inflammatory bowel disease and allergic dermatitis, all of which are conditions not mentioned as susceptible to therapy with the compounds disclosed in the Eisai patent publications. The present invention also provides methods for treating various disease conditions by administering a compound that inhibits more than a single kinase, particularly diseases and conditions where inhibiting a kinase in addition to MEKK, MEK1, VEGFR, and PDGFR, as well as a kinase other than MEKK (which, as noted, above, is not inhibited by a mechanism involving Michael adduct formation), would be expected to increase therapeutic efficacy. In other embodiments, the present invention provides methods for treating cancers resistant to certain drugs due to a mutation in a kinase other than MEKK, MEK1, VEGFR, and PDGFR by administering a compound described in the Eisai patent publications to inhibit that mutated kinase. In other embodiments of the methods of the invention, a compound other than a compound specifically described in the Eisai patent publications is administered to treat a disease or condition identified herein.

In another embodiment, the compounds and pharmaceutical compositions administered in the therapeutic methods of the invention are a subset of the compounds described in Cor Therapeutics, Inc., U.S. Pat. Nos. 5,674,892 (1997); 5,795,910 (1998); and 5,728,726 (1998); incorporated herein by reference. These publications recite that a variety of RALs, including those capable of forming the Michael adduct as described herein and those that are not, are generally useful as kinase inhibitors. Again, the absence of information about the effect of the compounds on other important kinases (only three kinases are even mentioned in the Cor patent publications), and the limited data available regarding the few kinases listed in these publications, makes assessment of the therapeutic potential of the compounds impossible from the Cor Therapeutics patents alone. The present invention teaches that those compounds disclosed in these Cor Therapeutics patents that are capable of Michael adduct formation as disclosed herein can be used to treat a variety of cancer indications and other diseases and conditions and provides data showing that the compounds target protein kinases in addition to those mentioned in the Cor Therapeutics patents. In other embodiments of the methods of the invention, a compound other than a compound specifically described in the Cor Therapeutics patents is administered to treat a disease or condition identified herein.

In another embodiment of the methods of the invention, a compound useful in a method of the invention is other than a compound selected from the group consisting of naturally occurring resorcylic acid lactones, hypothemycin, (5Z)-7-oxozeaneol, Ro-09-2210, and L-783,277, is administered to a patient in need of treatment for a disease or condition selected from the group consisting of AML, basal cell carcinoma, B-Raf mutation-dependent cancers including but not limited to colon cancers and melanoma, breast cancer, GI stromal tumors, Ras dependent cancers, renal cell carcinoma, prostate cancer, pulmonary fibrosis, mastocytosis, inflammatory bowel disease, and allergic dermatitis.

The following examples illustrate various methods for making, testing, and using compounds useful in the methods of the present invention.

### EXAMPLES

These examples describe the purification of hypothemycin and (5Z)-7-oxozeaneol from the fermentation of Hypomyces subiculosus ATCC 44392 or of Aigialus parvus. They show how enzyme kinetic analyses, using a lactone labeled with radioactivity, fluorescence, or biotin, or mass spectroscopy, can be used in demonstrating whether a compound (in this example, the illustrative compounds hypothemycin and (5Z)-7-oxozeaneol are used) forms covalent adducts with MEK1 or other Cys target kinases. In addition, these examples show how the ability of a lactone to inhibit a pathway of MAPK signaling can be determined by cell based assays, and how the anti-proliferation behavior of the lactone(s) can be demonstrated in cancer cells from ERK-dependent tumors in culture.

### Example 1

### Production of Resorcylic Acid Lactones

Hypothemycin or (5Z)-7-oxozeaneol can be purified from the fermentation of Hypomyces subiculosus ATCC 44392 following literature procedures. An alternative source of these and closely related resorcylic acid lactones, known as the aigialomycins, is the fermentation of the Agialus parvus strain. Other resorcylic acid lactone compounds of the invention can be synthesized, in accordance with this disclosure and methodology described in the literature. The structures of isolated compounds can be confirmed by NMR and MS analysis of the purified material. The ³H or ¹⁴C form of one of the lactones or analogs thereof can be prepared commercially (e.g. Moravek Biochemicals; Brea, Calif.) by a chemical or enzymatic semi-synthesis method and its structure verified by chromatographic and spectroscopic analysis. The present invention also provides a method for obtaining a mutant strain that produces (5Z)-7-oxozeaneol or 15-desmethyl hypothemycin instead of hypothemycin as follows. The biosynthetic gene cluster for hypothemycin is subcloned from a cosmid library made from the H. subiculosus genomic DNA after using end-sequencing to identify genes that encode the mono-modular type I polyketide synthase (PKS) and requisite tailoring enzymes. Candidate cosmids are sequenced until one(s) with the expected features are found, i.e., overlapping cosmids that contain the PKS gene plus at least one oxidase gene, an O-methyltransferase gene, and associated regulatory genes. Gene disruption is carried out to confirm that the correct set of biosynthesis genes had been identified. Finally, disruption of the oxidase gene results in production of (5Z)-7-oxozeaneol, the precursor of hypothemycin, or disruption of the O-methyltransferase gene results in production of 15-desmethyl hypothemycin. Compounds useful in the methods of the invention can also be prepared by total chemical synthesis (see Selles et al., Tetrahedron Lett, 2002; 43 (26):4621-5; Selles et al., Tetrahedron Lett 2002; 43 (26):4627-31; Geng et al., Org Lett 2004; 6 (3):413-6).

### Example 2

### Kinetic Analysis of Target Cys Kinase Inhibition by the Lactone

This example illustrates one method for demonstrating that a compound can form a Michael adduct with a target protein kinase, using MEK1, ERK2 and several mitogen receptor kinases as illustrative protein kinases. A hallmark of covalent adduct formation between an inhibitor and enzyme is “time-dependent inhibition” of enzyme activity.

Typically, one measures the increase in inhibition of protein kinase activity in the presence of inhibitor over time. In one method, aliquots of a “pre-incubation” reaction mixture containing enzyme and inhibitor are assayed for activity (initial velocities) over time; increased inhibition or decreased initial velocities will be observed over time as the Michael adduct forms (Walsh, C, Enzyme Reaction Mechanisms, W.H. Freeman & Co., 1979, pp 86-94). In a second method, the time dependent loss of activity is measured as “progress curves” that measure and analyze product formed (e.g. ADP) versus time (Morrison & Walsh, Adv. Enzymol Relat Areas Mol. Biol. 1988, 61, 201-301). In either case, the time dependent inactivation can be dampened by the presence of a competing substrate, in this case ATP.

The reversible dissociation constant, K_{d}, and the rate constant for inactivation, k_{inact}, values determined are the principal data used for analysis of the inhibition mechanism. Performance of these assays with hypothemycin plus its unreactive 5,6-dihydro form as controls demonstrates the importance of the α,β-unsaturated ketone for enzyme inhibition.

From the established mechanisms of other MEK1 inhibitors such as PD184352 and UO126, both of which act non-competitively with ATP, a lactone compound useful in the methods of the invention should inhibit the phosphorylation of ERK1 by MEK. Time-dependent enzyme inhibition may be seen with tight, slow-binding inhibitors or covalent bond-forming inhibitors and can be detected by the standard approaches described above.

MEK1 and many other protein kinases that can be targets of the compounds useful in the methods of the invention can be obtained commercially (Invitrogen; Carlsbad, Calif.) or prepared using standard molecular biology techniques. After activation by phosphorylation, they are assayed for their ability to phosphorylate a target kinase or surrogate substrate. For example, MEK1 can be assayed in a mixture containing MEK1 (30 nM) and ERK1 (2 μM), [γ-³²P]ATP (10 uM) and MgCl₂ in Mops buffer pH 7.6. Phosphorylation can be measured by isolating [γ-³²P]-phosphorylated ERK1 on phosphocellulose paper, and counting radioactive product. Alternatively, a coupled enzyme system may be used in which a product of the kinase reaction, such as ADP, is measured by analysis with a secondary system that converts that product (e.g. ADP) to an easily measurable entity (e.g. NADH); often, such coupled systems can be measured by convenient spectrophotometry assays.

To measure time-dependent inhibition using the “pre-incubation method”, MEK1 (or other kinase) is incubated with varying amounts of the lactone inhibitor; the control excludes the inhibitor or includes a competitive inhibitor (e.g. UO126, IC₅₀ 72 nM, obtainable from EMD Biosciences, San Diego, Calif.). Aliquots are removed at various times, added to a solution containing substrates [γ-³²P]ATP, ERK1 (or other substrate), and the other components of the reaction, and initial rates are determined as a measure of remaining enzyme activity.

For covalent inhibitors, there is a time-dependent loss of enzyme activity, whereas for reversible inhibitors the activity does not change over time (Morrison & Walsh, Adv. Enzymol Relat Areas Mol. Biol. 1988, 61:201-301; Sculley et al., Biochim Biopkys Acta 1996, 1298(1):78-86). For covalent inhibitors, plots of log (activity) versus time provide apparent first-order rate constants (k_{obsd}) of enzyme activity loss. If these assays are performed at varying concentrations of inhibitor, a series of first-order plots is obtained and k_{obsd} obtained at each inhibitor concentration. To measure time-dependent inhibition using the “progress-curve method” (see references cited above and Kuzmic et al., Methods Enzymol 2004; 383: 366-81), ERK (or other kinase) is treated with varying amounts of the lactone inhibitor, and the formation of ADP is measured continuously by a coupled assay. The resultant product versus time curves are fit to the equation: [P]=(vᵢ/k_{obs})*(1−exp(−k_{obs}*t) where P is the product formed at time t, vᵢ is the initial velocity and k_{obs} is the apparent first-order rate constant of inhibition, and k_{obs} values determined for each different inhibitor concentration. A re-plot of 1/k_{obsd} vs. 1/[I] allows determination of K_{d} (initial reversible binding constant) and k_{inact} (first-order rate constant for conversion of reversibly-bound E-I to covalently-bound E-I), which can be used to calculate the half-life of inactivation by dividing it into 0.693. Control experiments are performed with analogs of hypothemycin that do not have an α,β-unsaturated carbonyl (e.g. 5,6-dihydro hypothemycin) and hence cannot form a Michael adduct. Such molecules may be competitive inhibitors but should not show time-dependent inactivation.

Table 2 shows the relevant inhibition constants for hypothemycin against several kinases, including, where relevant, kinetic constants for time-dependent inactivation. The parameters differ significantly for different kinases, and the over 100-fold differences in “selectivity constants” (k_{inact}/Kᵢ) suggest that kinases such as KDR (VEGFR) and MEK1 can be inhibited selectively over others by using a low concentration×time (dose×exposure in cells or organism). Those of skill in the art will recognize that within the set of compounds useful in the methods of the invention, significant variability in specificity can be achieved, allowing one to identify optimal compounds for different applications. Inhibition progress curve analysis was performed using continuous, colorimetric, or fluorimetric assays. All experiments were done at 1 mM ATP, with the exception of KDR (at 5 mM ATP).

TABLE 2
Inhibition data of resorcylic acid lactones against various protein kinases

| | Kinase | Kᵢ | k_{inact} | T_{1/2} | k_{inact}/Kᵢ |
|---|---|---|---|---|---|
| Kinase | Inhibitor | (M) | (sec⁻¹) | (sec) | (M⁻¹ sec⁻¹) |
| MEK1 | hypothemycin | 1.9E−08 | 0.003 | 277 | 1.3E+05 |
| MEK1 | 5,6-dihydro- | 3.0E−06 | None | None | None |
| | hypothemycin | | | | |
| ERK2 | hypothemycin | 2.7E−06 | 0.005 | 139 | 1.9E+03 |
| ERK2 | 5,6-dihydro- | 2.8E−05 | None | None | None |
| | hypothemycin | | | | |
| Flt-3 | hypothemycin | 1.5E−07 | 0.007 | 99 | 4.7E+04 |
| Flt-1 | hypothemycin | 1.1E−07 | 0.018 | 39 | 1.6E+05 |
| (VEGFR1)^{a} | | | | | |
| KDR | hypothemycin | 1.4E−08 | 0.007 | 99 | 5.0E+05 |
| (VEGFR2) | | | | | |
| PDGFRα | hypothemycin | 1.5E−06 | 0.002 | 347 | 1.3E+03 |
| PDGFRβ | hypothemycin | 1.2E−06 | 0.003 | 231 | 2.5E+03 |
| TrkA | hypothemycin | 2.2E−06 | None | None | None |
| TrkB | hypothemycin | 3.7E−07 | None | None | None |
| GSK3α^{b,c} | hypothemycin | >6.3E−04 | nd | nd | nd |
| GSK3β^{c} | hypothemycin | 3.7E−04 | 0.002 | 350 | 5.40E+00 |

^{a}Significant decrease of enzyme activity over time without inhibitor present
^{b}5% Inhibition assuemd at highest inhibitor concentration
^{c}Kᵢ calculated from % inhibition (initial rate) at highest inhibitor concentration.

The results shown in Table 3, below, demonstrate that hypothemycin does not significantly inhibit kinases lacking the critical Cys residue and does inhibit, to varying degrees, kinases having it. In this panel of 124 kinases, 18 of the 19 kinases identified as having an active site cysteine were inhibited to some extent by hypothemycin at a concentration of 0.2 μM and/or 2 μM. Of the non-active site cysteine kinases, only two showed significant inhibition by hypothemycin. (These values may differ from those attainable in a different assay, i.e., with different sample handling techniques, because they are single point assays that do not take into account the time-dependent nature of covalent inhibition).

TABLE 3
Percent residual activity of protein kinases treated with
hypothemycin (0.2 μM or, if in parentheses, 2.0 μM)

| Kinase | % activity |
|---|---|
| Abl(h) | 98 |
| Abl(T315I)(h) | 90 |
| ALK(h) | 101 |
| Arg(h) | 98 |
| ASK1(h) | 100 |
| Aurora-A(h) | 87 (91) |
| Axl(h) | 110 |
| Bmx(h) | 99 |
| BRK(h) | 89 |
| BTK(h) | 104 |
| CaMKIV(h) | 102 |
| CDK1/cyclinB | 89 |
| (h) | |
| CDK2/cyclinA | 634 (106) |
| (h) | |
| CDK2/cyclinE | 105 |
| (h) | |
| CDK3/cyclinE | 95 |
| (h) | |
| CDK5/p35(h) | 103 |
| CDK6/cyclinD | 95 |
| 3(h) | |
| CDK7/cyclinH/ | 101 |
| MAT1(h) | |
| CHK1(h) | 94 |
| CHK2(h) | 97 |
| CK1δ(h) | 105 |
| CK2(h) | 99 |
| cKit(h)^{a} | 93 (21) |
| cKit(D816V) | 57 (0) |
| (h)^{a} | |
| c-RAF(h) | 103 |
| CSK(h) | 104 |
| cSRC(h) | 75 (49) |
| DDR2(h) | 92 |
| EGFR(h) | 106 (94) |
| EphA2(h) | 87 |
| EphB2(h) | 118 |
| EphB4(h) | 82 |
| ErbB4(h) | 124 |
| Fer(h) | 102 |
| Fes(h) | 92 |
| FGFR1(h) | 99 |
| FGFR3(h) | 102 |
| FGFR4(h) | 87 |
| Fgr(h) | 100 |
| Flt1(h)^{a} | 2 (7) |
| Flt3(h)^{a} | 6 (3) |
| Flt3(D835Y) | 4 (2) |
| (h)^{a} | |
| Fms(h) | 91 (90) |
| Fyn(h) | 87 |
| GSK3α(h)^{a} | 90 (88) |
| GSK3β(h)^{a} | 90 (40) |
| Hck(h) | 78 (91) |
| IGF-1R(h) | 107 |
| IKKα(h) | 101 |
| IKKβ(h) | 81 (94) |
| IR(h) | 100 |
| IRAK4(h) | 84 |
| JNK1α1(h) | 97 |
| JNK2α2(h) | 93 |
| JNK3(h) | 76 |
| KDR^{a} | nt (15) |
| Lck(h) | 98 |
| Lyn(h) | 82 (83) |
| MAPK1(h)^{a} | 79 (10) |
| MAPK2(h)^{a} | 79 (5) |
| MAPKAP- | 98 |
| K2(h) | |
| MAPKAP- | 98 |
| K3(h) | |
| MEK1(h)^{a} | 54 (8) |
| Met(h) | 96 |
| MINK(h) | 88 |
| MKK6(h)^{a} | 43 (8) |
| MKK7β(h)^{a} | 85 (41) |
| MSK1(h) | 87 |
| MSK2(h) | 101 |
| MST2(h) | 94 |
| NEK2(h) | 99 |
| NEK6(h) | 104 |
| NEK7(h) | 98 |
| p70S6K(h) | 94 |
| PAK2(h) | 104 |
| PAK4(h) | 92 |
| PAR-1Bα(h) | 93 |
| PDGFRα(h)^{a} | 77 (20) |
| PDGFRβ(h)^{b} | 73 (40) |
| PDK1(h) | 93 |
| Pim-1(h) | 100 |
| PKA(h) | 108 |
| PKBα(h) | 153 |
| PKBβ(h) | 91 |
| PKBγ(h) | 102 |
| PKCα(h) | 96 |
| PKCβI(h) | 99 |
| PKCβII(h) | 97 |
| PKCγ(h) | 98 |
| PKCδ(h) | 96 |
| PKCε(h) | 106 |
| PKCη(h) | 97 |
| PKCι(h) | 108 |
| PKCμ(h)^{a} | 34 (6) |
| PKCθ(h) | 104 |
| PKCζ(h) | 94 |
| PKD2(h)^{a} | 31 (−2) |
| Plk3(h) | 101 |
| PRAK(h)^{a} | 20 (1) |
| PRK2(h) | 108 |
| Pyk2(h) | 84 |
| Ret(h) | 104 |
| RIPK2(h) | 101 |
| ROCK-I(h) | 121 |
| ROCK-II(h) | 104 |
| Ron(h) | 91 |
| Ros(h) | 97 |
| Rse(h) | 90 |
| Rsk1(h) | 100 |
| Rsk2(h) | 91 |
| Rsk3(h) | 94 |
| SAPK2a(h) | 107 |
| SAPK2b(h) | 104 |
| SAPK3(h) | 104 |
| SAPK4(h) | 101 |
| SGK(h) | 99 |
| Syk(h) | 105 |
| TAK1(h)^{a} | 12 (5) |
| Tie2(h) | 93 |
| TrkA(h) | 22 (1) |
| TrkB(h) | 58 (18) |
| Yes(h) | 99 |
| ZAP-70(h) | 111 |
| ZIPK(h) | 96 |

^{a}Has active site cysteine

MEK1 assays were performed using pre-incubation experiments with radioactive [³²P]ATP and filter binding of product. All other kinases were analyzed using progress curve analysis from a continuous spectrophotometric assay.

TRKA and B showed inhibition by hypothemycin in the single point screening assay described above (Table 3), but do not contain the target Cys for Michael adduct formation. When these enzymes were assayed by this more exact method, hypothemycin showed reversible inhibition competitive with ATP with a Kᵢ of 2.2 μM for TRKA and 0.37 μM for TRKB, but did not show time-dependent inactivation (i.e. covalent bond formation) of the enzymes (Table 2); this verifies that covalent inhibition requires the target Cys residue and validates time dependent inhibition as a criteria for covalent enzyme inhibition of the target kinases.

### Example 3

### Determination of Covalent Bond Formation

In a Michael reaction, which is in principle a reversible reaction, the apparent affinity between free and covalently-bound ligand is the product of the two dissociation constants (K_{reversible}×K_{covalent}) involved in the reaction, and formation/disruption of the complex is in theory reversible because the protein catalyzes reactions in both directions. Denaturation of the protein obliterates catalysis in both directions, and denatured Michael adducts are usually sufficiently stable that they can be physically isolated and quantitated. For example, although native FdUMP-thymidylate synthase Michael adducts are slowly but completely reversible, SDS denaturation provides stable, isolable complexes (D. V. Santi et al., Biochemistry, 1914, 13, 471; Methods in Enzymol. 1977, 46, 307-312.

Of course, if the complex does not involve a covalent adduct, denaturation of the protein results in immediate dissociation of the inhibitor. Thus, a number of Michael adducts have been isolated simply by denaturing a [³H]-ligand-protein complex and detecting protein-bound radioactivity by SDS-PAGE. The detection of such complexes provides the following: (a) evidence of covalent adduct formation, and (b) a tool for quantitating the interaction to determine equilibrium (K_{d}) and kinetic constants (k_{off} and kₒₙ).

For example, the various available forms of MEK1 or other targeted Cys-containing kinases can be treated with fluorescent or [³H]-hypothemycin or analogous analogs, subjected to SDS-PAGE or a denaturing gel permeation column, and the gels or column analyzed for protein-bound fluorescence or radioactivity. If stable complexes form, a number of important tests can be performed. For example, the complex can be isolated from SDS-PAGE, digested with trypsin, and the covalently bound peptides of the protein identified by chromatographic or mass spectral (MS) analysis. The equilibrium and kinetic properties of complex formation can be determined by varying the concentration of [³H] or fluorescently-labeled enone and isolating/quantitating the complex by SDS-PAGE. Cultured mammalian cells or soluble cell extracts obtained from such cells can be treated with [³H]-labeled or fluorescently-labeled hypothemycin, analyzed on 2D gels, and the protein in radio-active spots identified by MALDI MS. If, for example, MEK1 were the sole target for covalent adduct formation with hypothemycin, MEK1 will be the only protein labeled; if multiple proteins are labeled, one can conclude there are additional targets and identify them.

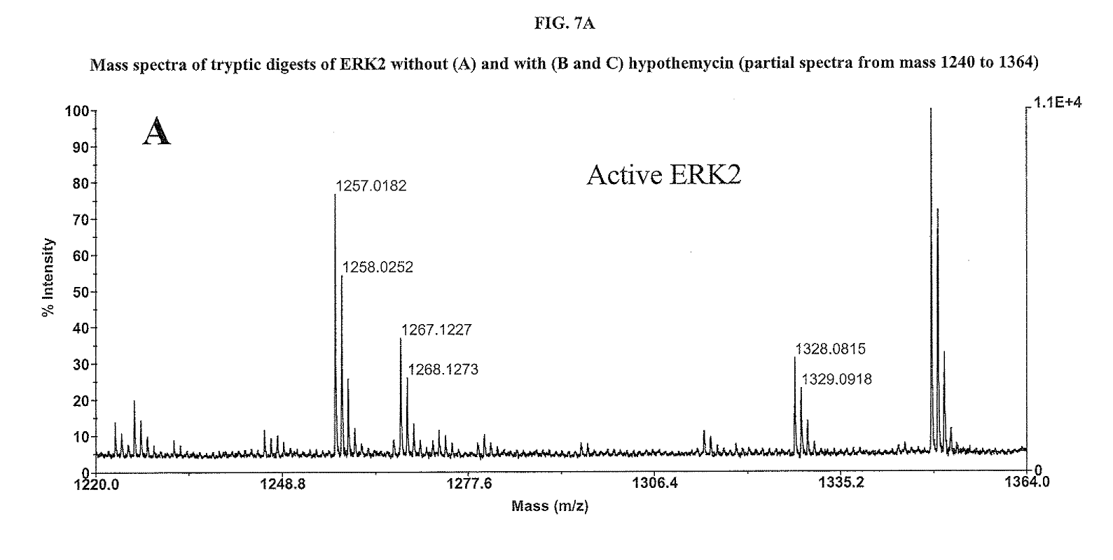

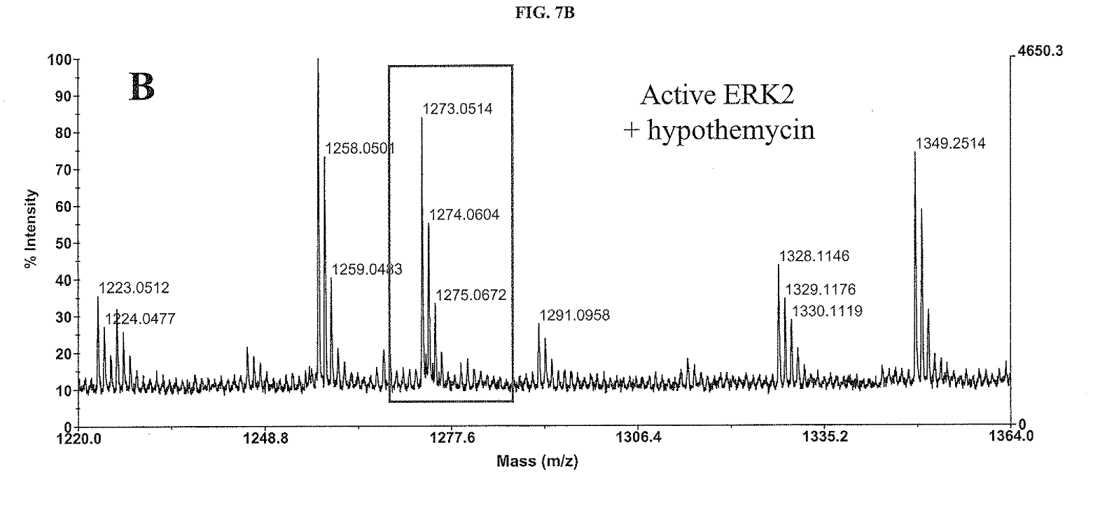

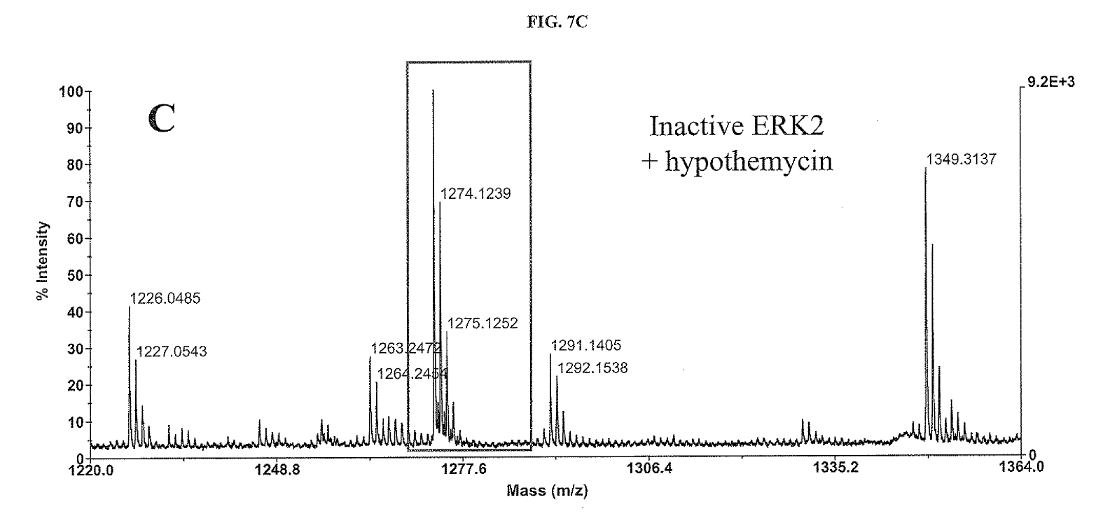

For example, covalent bond formation to the critical cysteine of a kinase can be demonstrated by mass spectral analysis of peptides obtained by proteolytic digestion of the covalent complex. FIG. 7 shows the mass spectra of tryptic digests of ERK2 with and without hypothemycin. A mass peak of 951 corresponds to the mass of the smallest tryptic peptide containing the target Cys 172 residue. The tryptic digests of the unactivated and activated forms of ERK2 previously treated with hypothemycin show that the mass of the target Cys peptides is increased by 1273, an amount that exactly equals that of hypothemycin.

### Example 4

### Inhibition of the Proliferation of Cells Cultured from Cys Kinase-Dependent Cancers by the Lactone

The ability of the compounds useful in the methods of the invention to inhibit cell proliferation of cell lines derived from tumors that involve active signaling pathways that possess or are activated by protein kinases containing the active site Cys residue susceptible to Michael adduct formation can be demonstrated using cell proliferation assays and cell lines such as HT-29 (human colon carcinoma), COLO829 (melanoma), MV-4-11 (acute myelogenous leukemia) and P815 (mouse mastocytoma). In one illustrative method, cells are treated with various concentrations of the inhibitor in 96 well plates, incubated at 37° C./5% CO₂ for three days, and analyzed using the Cell Titer Glo kit (Promega).

Table 4 shows the growth inhibitory properties of compounds useful in the methods of the invention against cell lines that involve active signaling pathways that possess or are activated by protein kinases containing the active site Cys residue susceptible to Michael adduct formation derived from tumors. Shown are the mutant kinase from which the disease sensitivity is primarily derived, as well as other protein kinase targets of hypothemycin rationally identified a priori that contribute to sensitivity.

TABLE 4
Cytotoxicity of Kinase Inhibitors in Kinase Mutant Cell Lines

| Cell Line | Kinase Inhibitor (IC₅₀, μM) | | | | | |
|---|---|---|---|---|---|---|
| (Cancer type, | RAL-targeted | | | | BAY | PD |
| kinase mutation) | kinases) | Hypothemycin | 5,6-Dihydrohypothemycin | SU11248 | 43-9006 | 98059 |
| A549 | | 6 | 107 | — | 5.5 | 48 |
| (NSCLC, B-Raf | | | | | | |
| wild type) | | | | | | |
| HT29 | MEK½ | 0.1 | 15 | 4.2 | 4.7 | 5.5 |
| (Human colon, | ERK½ | | | | | |
| B-Raf V599E) | | | | | | |
| DU4475 | MEK½ | 0.018 | 46 | 4.0 | 3.6 | 56 |
| (Human breast, | ERK½ | | | | | |
| B-Raf V599E) | | | | | | |
| WM266-4 | MEK½ | 0.04 | 15 | 8.2 | 5.4 | 21 |
| (Human melanoma, | ERK½ | | | | | |
| B-Raf V599D) | | | | | | |
| COLO829 | MEK½ | 0.089 | 3.7 | 7.1 | 6.0 | — |
| (Human melanoma, | ERK½ | | | | | |
| B-Raf V599E) | | | | | | |
| A375 | MEK½ | 0.18 | >50 | 5.4 | 4.3 | 43 |
| (Human Melanoma, | ERK½ | | | | | |
| B-Raf V599E) | | | | | | |
| P815 | KIT | 0.31 | — | 0.32 | 0.31 | — |
| (Mouse mastocytoma, | MEK½ | | | | | |
| KIT D814Y) | ERK½ | | | | | |
| MV4-11 (Human | Flt3 | 0.0055 | — | 0.010 | 0.0023 | — |
| leukemia Flt3- | MEK½ | | | | | |
| ITD)) | ERK½ | | | | | |
| EOL-1 (Human | PDGFR | 0.00041 | — | 0.0017 | 0.00023 | — |
| leukemia FLP1L1- | MEK½ | | | | | |
| PDGFRA) | ERK½ | | | | | |
| ASZ001 | PDGFR | 0.10* | — | — | — | — |
| (Basal cell | MEK½ | | | | | |
| carcinoma) | ERK½ | | | | | |

*10 for Tazarotene

### Example 5

### Effects of the Lactone Inhibitor on Signaling Pathways in Whole Cells

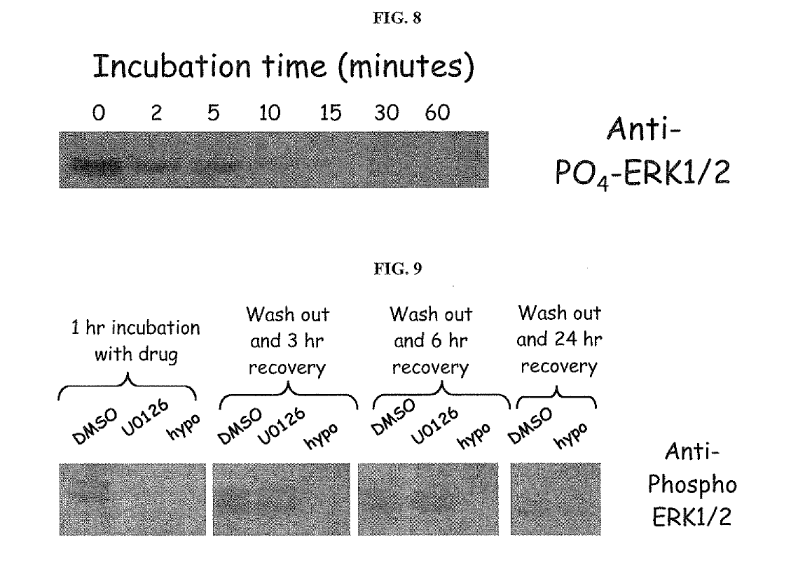

The downstream effects of inhibition of a particular kinase (e.g. MEK) can be established by measuring the phosphorylation state of several proteins that require that kinase for phosphorylation (e.g. ERK1). Cultured cells are treated with hypothemycin or other lactone analogs described herein, and Western blots of cell extracts are probed with antibodies specific for the unmodified and phosphorylated forms of the downstream targets. As an example, the effects of hypothemycin on MEK1/2 can be determined by measuring the level of ERK1/2 phosphorylation. FIG. 8 shows that treatment of COLO829 cells (containing the BRAFV599E mutation) with hypothemycin rapidly (within 10 minutes) results in the depletion of the phosphorylated form of ERK. Likewise, treatment of a cell containing high levels of a mitogen receptor kinase target of hypothemycin, such as MV-4-11, which has the FLT3(ITD) mutation, results in the loss of phosphorylated forms of FLT3 as well as both of the downstream targets of the receptor tyrosine kinase MEK and ERK.

Referring to FIG. 8, B-Raf V599E mutant melanoma cell line COLO829 was incubated with 1 microM hypothemycin for 2, 5, 10, 15, 30, and 60 minutes. The cells were then lysed and the proteins extracted. Equal amounts of total protein from each sample were separated by SDS-PAGE followed by electroblotting to a PVDF membrane. The levels of phospho-ERK present in each extract were visualized by incubation of the membrane with anti-phosphoERK antibody (Cell Signaling Technologies) followed by incubation with an HRP linked secondary antibody. Phospho-ERK containing bands were detected by autoradiography using the ECL Western detection kit (Amersham). Reprobing of this blot with ERK antibodies demonstrated that equal levels of total ERK were loaded in each lane (data not shown).

As with reversible inhibitors, the effect of inhibiting target Cys kinases, as measured by phosphorylation of effected downstream kinases, is rapidly accomplished. However, unlike reversible competitive inhibitors, and as shown in FIG. 9, the lactone may be removed from cells after a brief exposure of one hour or less and the inhibited kinase does not recover for long periods of time (up to 24 hr). Thus, in cells as in vitro, the covalent inhibitor-kinase adduct forms rapidly and remains bound for long periods of time. Thus, an unusual attribute of these inhibitors as drugs is that a short exposure of the drug to the target can have a long duration of effect, which provides desirable options in terms of scheduling to achieve maximal efficacy while avoiding toxicities due to off-target effects. This also means that RALs with relatively short in vivo half-lives can be effectively employed in the methods of the invention, provided the dose and the half-life are sufficient to ensure significant inhibition of the target kinase(s).

Referring to FIG. 9, B-Raf V599E mutant cell line HT29 was incubated with either DMSO, 1 μM U0126, or hypothemycin for 1 hour. Following the 1 hour incubation, cells were then washed twice with media and incubated. Protein extracts were prepared immediately following drug treatment and at 3, 6, and 24 hours post-wash. Equal amounts of total protein from each sample were separated by SDS-PAGE followed by electroblotting to a PVDF membrane. The levels of phospho-ERK present in each extract were visualized by incubation of the membrane with anti-phosphoERK antibody (Cell Signaling Technologies) followed by incubation with HRP linked secondary antibody. Phospho-ERK containing bands were detected by autoradiography using the ECL Western detection kit (Amersham).

Prior to drug development, the pharmacokinetics, bioavailability, antitumor activity in animals and acute toxicity of a compound is conducted. Based on existing knowledge about resorcylic acid lactones, compounds useful in the methods of the invention are not highly toxic and should have good bioavailability. Patient typing for mutant alleles predicting sensitivity to such drugs is also conducted in some embodiments (e.g. B-Raf mutations in malignant melanoma), as exemplified by a recent study of the treatment of lung cancer patients with Iressa.

### Example 6

### Preparation and Properties of a Compound of this Invention

This example describes the preparation of a compound of this invention, namely 4-O-desmethylhypothemycin, having a structure according to formula II. In particular, this compound is provided in its purified and isolated form.

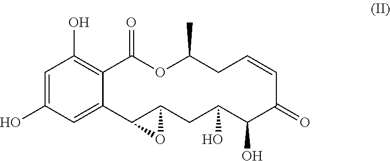

Innoculum preparation. One milliliter of frozen cells of Hypomyces subiculosus DSM 11931 from the Deutsche Sammlung von Mikroorganismen und Zellkulturen (DSMZ) maintained, in 20% (v/v) glycerol was inoculated into 50 mL of seed medium in a unbaffled Erlenmeyer flask. The seed medium consisted of 30 g/L Quaker oatmeal in water and was heated to 70-80° C. for 10 min before autoclaving. The seed culture was incubated in the dark at 22° C. and 190 rpm on a rotary shaker with a 2-inch stroke for 3 days. Secondary seed cultures were generated by transferring 2 mL of the primary seed culture into 50-mL unbaffled Erlenmeyer flasks containing 50 mL of oat flake medium. These cultures were grown at 22° C. and 190 rpm for 2 days.

Fermentor production. A 20-L bioreactor (New Brunswick) containing 12 L of CYS80 medium (Dombrowski et al., J Antibiot, 1999, 52 (12), 1077-1085), consisting of 80 g/L sucrose, 50 g/L corn meal (Sigma), and 1 g/L Bacto yeast extract (BD), was sterilized-in-place at 121° C. for 30 min. The medium was then inoculated with 480 mL of H. subiculosus DSM 11931 secondary seed culture. The fermentation was performed at 22° C. with an aeration rate of 0.4 v/v/m and an initial agitation rate of 200 RPM. The culture dissolved oxygen was controlled at 30% of air saturation by an agitation cascade between 200-400 RPM. Foaming was controlled by the automatic addition of 100% UCON LB-625. The culture pH was monitored but not controlled. D,L-ethionine was added to the production culture at a concentration of 50 mg/L at the time of inoculation. The fermentation continued for 35 days until maximum KOSN-2176 production was reached. Samples were withdrawn as necessary and stored at −20° C. for later analysis.

Those skilled in the art will appreciate that variations in the composition of the CYS80 culture medium are usable, for example, it can contain between about 30 and about 120 g/L sucrose, between about 20 and about 80 g/L corn meal, and about 0 (preferably about 0.1) to about 10 g/L yeast extract. Similarly, the D,L-ethionine concentration can vary, for instance between about 10 and about 100 mg/L of culture medium.

To promote the accumulation of compound II, various compounds were evaluated as inhibitors of the methyltransferase responsible for catalyzing the methylation of the C-4 hydroxyl group to produce hypothemycin. D,L-Ethionine, which had been reported in the literature to be a methyltransferase inhibitor, was found to be effective in increasing the production of compound II, while other reported methyltransferase inhibitors did not. Also, a number of culture media were evaluated, with CYS80 being more conducive to compound II production than the others. Titers of compound II were improved from 40 mg/mL to 540 (20-liter bioreactor) to 900 mg/mL (shake flask).

Quantitation of compound II. The production of compound II and hypothemycin was monitored by extracting 500 μL of fermentation broth with 1 mL of methanol. The mixture was then centrifuged at 13,000 g for 3 min. Quantitation of the two products in the supernatant was performed using a Hewlett Packard 1090 HPLC with UV detection at 220, 267, and 307 nm. Five microliters of the supernatant were injected across a 4.6×10 mm guard column (Inertsil, ODS-3, 5 μm) and a longer 4.6×150 mm column (Inertsil, ODS-3, 5 μm). Samples were diluted with methanol until the final hypothemycin concentration was less than 1 g/L. The assay method was performed at a flow rate of 1 mL/min at ambient temperature. It consisted of a gradient from 40:60 to 80:20 acetonitrile:water over 8 min, followed by a 100% acetonitrile wash for 4 min. Both mobile phases contained 0.1% (v/v) acetic acid. Standards were prepared using purified compound II and hypothemycin.

Purification of compound II. Twenty-four liters of fermentation broth from two 12-L fermentations of H. subiculosus DSM 11931 were extracted with 24 L of 100% methanol for 1 h. The mixture was passed through a vacuum filter with a thin layer of Celite (Hyflo), and the filter cake was washed with 1 L of 50:50 methanol:water. The filtrate was diluted with water to a final methanol concentration of 30% (v/v). All the solvents used in the purification process contained 0.1% (v/v) acetic acid.

A Millipore Moduline (50 cm×9 cm) process column was packed with 1.3 L of HP-20SS resin (Mitsubishi) and equilibrated, with 3 column volumes (CV) of 30:70 methanol:water at 700 mL/min. The product pool was loaded onto the column at the same flow rate. The column was washed with 1 CV of 30:70 methanol:water and eluted with a step gradient (3 CV of 45:55 methanol:water, 9 CV of 50:50 methanol:water, and 3 CV of 60:40 methanol:water) at 300 mL/min. Fractions (1.5 CV) were collected and analyzed by HPLC as described above. Fractions 3-15 were combined as the product pool.

A Millipore Moduline (50 cm×9 cm) process column was packed with 2.3 L of C₁₈ sorbent (Bakerbond, 40 μm) and equilibrated with 3 CV of 30:70 methanol:water at 180 mL/min. The product pool from the HP-20SS chromatography step was diluted with water to a final methanol concentration of 30:70 methanol:water and loaded onto the C₁₈ column at 180 mL/min. The column was washed with 1 CV of 30:70 methanol:water and eluted with 9 CV of 42:58 methanol:water at 180 mL/min. Fractions (0.4 CV) were collected and analyzed by HPLC as described above. Fractions 10-16 were combined as the product pool.

To promote the crystallization of compound II, the product pool was concentrated by rotary evaporation at 40° C. to reduce its volume by 36%. It was then cooled, to −20° C. White crystals of compound II that were formed were filtered through a Buchner funnel with a Whatman #5 filter paper and washed with 100 mL of chilled water. The final product was dried in a vacuum oven at 40° C. overnight and stored at 4° C. The overall yield of the purification process was approximately 60%. The purity of compound II at the end of the purification process was approximately 95%.

Characterization of compound II. Purified, compound II was obtained as white crystals; UV (MeOH) λₘₐₓ 219, 266, 306 nm; HRESIMS m/z 363.1074 [M−H]⁻ (Calcd for C₁₈H₁₉O₈, 363.1065); ¹H and ¹³C NMR data, see Tables 5 and 6.

TABLE 5
¹H NMR of Compound II

| Proton | δ (ppm) | J (Hz) |
|---|---|---|
| 3 | 6.18 | s |
| 5 | 6.18 | s |
| 1′ | 4.27 | d, 1.5 |
| 2′ | 2.68 | dt, 9.5, 1.5 |
| 3′a | 0.93 | dd, 14.5, 9.5 |
| 3′b | 1.83 | dd, 14.5, 10.0 |
| 4′a | 3.84 | dd, 10.0, 5.5 |
| 4′b | — | |
| 5′ | 4.43 | dd, 5.0, 1.5 |
| 6′ | — | |
| 7′ | 6.40 | dd, 11.5, 3.0 |
| 8′ | 6.06 | td, 11.5, 2.5 |
| 9′a | 2.46 | m |
| 9′b | 2.88 | dt, 17.0, 11.0 |
| 10′ | 5.34 | m |
| 10′-CH₃ | 1.31 | d, 6.0 |
| 2-OH | 11.89 | s |
| 4-OH | 10.46 | br |
| 4′-OH | 5.15 | d, 6.5 |
| 5′-OH | 4.88 | d, 5.0 |
| 6′-OH | — | |

TABLE 6
¹³NMR of Compound II

| Carbon | δ (ppm) |
|---|---|
| 1 | 102.8 |
| 2 | 165.1 |
| 3 | 102.3 |
| 4 | 163.5 |
| 5 | 103.9 |
| 6 | 142.9 |
| 1′ | 56.7 |
| 2′ | 63.1 |
| 3′ | 33.5 |
| 4′ | 68.9 |
| 5′ | 81.1 |
| 6′ | 201.2 |
| 7′ | 128.1 |
| 8′ | 142.4 |
| 9′ | 36.1 |
| 10′ | 73.8 |
| —COO— | 170.8 |
| 10′-CH3 | 20.4 |

### Example 7

### Synthesis of Compounds

This example describes the synthesis of additional compounds usable in the methods of this invention.

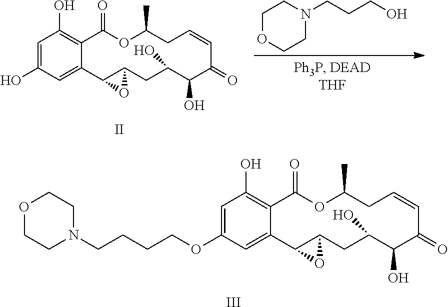

To a stirred solution of compound II (12 mg, 0.033 mmol) in THF (4.0 mL) was added 3-morpholinopropan-1-ol (10.0 μL, 0.072 mmol), triphenylphosphine (22.6 mg, 0.086 mmol) and diethyl azodicarboxylate (13.4 μL, 0.086 mmol). After stirring at room temperature for 3 h, the reaction mixture was concentrated. The residue was dissolved in THF/water (3:2, 1.2 mL), passed through a 0.45 μm filter, and purified by HPLC on a Varian Inertsil® 5μ ODS-3 (250×100) reverse-phase HPLC column. Elution with 10% to 90% gradient of 0.1% AcOH in water/0.1% AcOH in CH₃CN over 40 min provided compound III (11 mg, 70% yield): LRMS m/z (M+H) calcd for C25H34NO9 492.2; obsd 492.2.

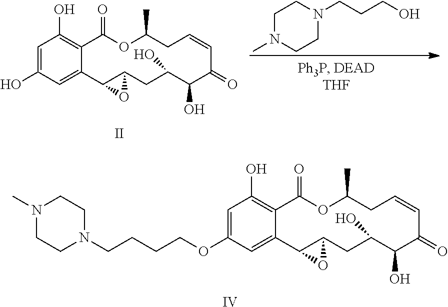

To a stirred solution of compound II (6 mg, 0.017 mmol) in THF (2.0 mL) was added 3-(4-methylpiperazin-1-yl)propan-1-ol (5.0 μL, 0.036 mmol), triphenylphosphine (12 mg, 0.043 mmol) and diethyl azodicarboxylate (7.0 μL, 0.043 mmol). After stirring at room temperature for 45 min, the reaction mixture was concentrated. The residue was dissolved in THF/water (3:2, 0.8 mL), passed through a 0.45 μm filter, and purified by HPLC on a Varian Inertsil® 5μ ODS-3 (250×100) reverse-phase HPLC column. Elution with 10% to 90% gradient of 0.1% AcOH in water/0.1% AcOH in CH₃CN over 40 min provided compound IV (3.8 mg, 45% yield): LRMS m/z (M+H) calcd for C26H37N2O8 505.2; obsd 505.2.

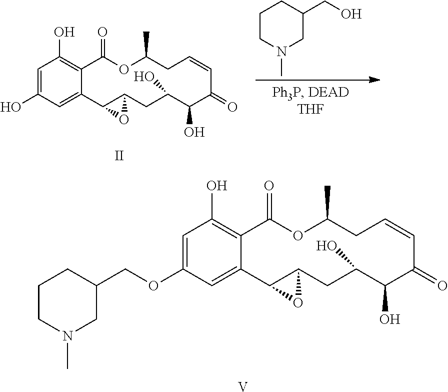

To a stirred solution of compound II (3 mg, 0.009 mmol) in THF (1.0 mL) was added (1-methylpiperidin-3-yl)methanol (5.0 μL, 0.036 mmol), triphenylphosphine (16 mg, 0.048 mmol) and diethyl azodicarboxylate (6.3 μL, 0.048 mmol). After stirring at room temperature for 3 h, the reaction mixture was concentrated. The residue was dissolved in THF/water (3:2, 0.8 mL), passed through a 0.45 μm filter, and purified by HPLC on a Varian Inertsil® 5μ ODS-3 (250×100) reverse-phase HPLC column. Elution with 10% to 90% gradient of 0.1% AcOH in water/0.1% AcOH in CH₃CN over 40 min provided IV (1.7 mg, 40% yield): LRMS m/z (M+H) calcd for C₂₅H34NO8 476.2; obsd 476.2.

The biological properties of compounds II-V were assayed and compared against those of hypothemycin. COLO829 is a human melanoma cell line. HT29 is a human colon cancer cell line. Both cell lines have a V600E B-Raf mutation. SKOV3 is an ovarian cancer cell line having wild-type B-Raf. EKR2 (extra-cellular signal regulated kinase 2) is a kinase in the Ras/B-Raf MAP kinase cascade pathway. The results are presented in Table 7.

TABLE 7
Properties of Compounds II-V

| | Cell Line (IC₅₀, μM) | | | ERK2 Inhibition | |
|---|---|---|---|---|---|
| Compound | COLO829 | HT29 | SKOV3 | Kᵢ (μM) | k_{inact.} (sec⁻¹) |
| Hypothemycin | 0.073 ± 0.017 | 0.24 ± 0.17 | ~4 | 1.9 ± 1.1 | 5 ± 2 × 10⁻³ |
| | N = 9 | N = 13 | | | |
| II | 0.038 | 0.10 | 1.8 | 2.2 ± 0.4 | 3.3 ± 0.4 × 10⁻³ |
| III | 0.047 ± 0.008 | 0.29 ± 0.21 | 0.86 ± 0.06 | 19.9 ± 7.8 | 3.3 ± 0.9 × 10⁻³ |
| | N = 2 | N = 2 | N = 2 | | |
| IV | 0.042 ± 0.025 | 0.21 ± 0.02 | Not tested | 3.1 ± 1.6 | 5 ± 1 × 10⁻³ |
| | N = 2 | N = 2 | | | |
| V | 0.079 | 0.60 | 15 | 0.94 ± 0.58 | 1 ± 0.1 × 10⁻³ |
| | N = 1 | N = 1 | N = 1 | | |

The invention having now been described by way of written description and examples, those of skill in the art will recognize that it can be practiced in a variety of embodiments and that the foregoing description and examples are for purposes of illustration and not limitation of the following claims.

What is claimed is:

1. A method for inhibiting one or more protein kinases in a mixture or cell, wherein said one or more protein kinases have a cysteine residue (Cys) located between two and immediately adjacent to one of two conserved aspartate residues in the ATP-binding site of said protein kinase, wherein said mixture comprises additional protein kinases lacking a Cys residue located between two and immediately adjacent to one of two conserved aspartate residues in an ATP-binding site of said additional protein kinases, said method comprising contacting said kinase with a compound capable of forming a Michael adduct with said Cys residue in said one or more protein kinases under conditions such that said Michael adduct forms between said compound and said Cys residue and results in inhibition of said one or more protein kinases,
wherein said compound has a structure I 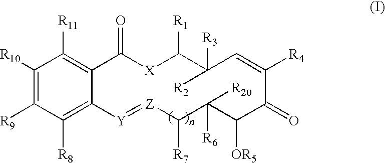
wherein
R₁ is hydrogen or an optionally substituted aliphatic, optionally substituted cycloaliphatic, optionally substituted heterocycloaliphatic, optionally substituted aryl, or optionally substituted heteroaryl moiety;
R₂ and R₃ are each independently hydrogen, halogen, hydroxyl, protected hydroxyl, or an optionally substituted aliphatic, optionally substituted cycloaliphatic, optionally substituted heterocycloaliphatic, optionally substituted aryl or optionally substituted heteroaryl moiety; or R₁ and R₂, when taken together, form an optionally substituted, saturated or unsaturated cyclic ring of 3 to 8 carbon atoms; or R₁ and R₃, when taken together, form an optionally substituted, saturated or unsaturated cyclic ring of 3 to 8 carbon atoms;
R₄ is hydrogen or halogen;
R₅ is hydrogen, C₂ to C₄ alkyl, an oxygen protecting group or a prodrug moiety;
R₆ is hydrogen, hydroxyl, or protected hydroxyl;
n is 0, 1, or 2;
R₇ is, for each occurrence, independently hydrogen, hydroxyl, or protected hydroxyl;
R₈ is hydrogen, halogen, hydroxyl, protected hydroxyl, alkoxy, or an aliphatic moiety optionally substituted with hydroxyl, protected hydroxyl, SR₁₂, or NR₁₂R₁₃;
R₉ is hydrogen, halogen, hydroxyl, protected hydroxyl, OR₁₂, SR₁₂, NR₁₂R₁₃,
—X₁(CH₂)ₚX₂—R₁₄, or is alkyl optionally substituted with hydroxyl, protected hydroxyl, halogen, amino, protected amino, or —X₁(CH₂)ₚX₂—R₁₄;
wherein
R₁₂ and R₁₃ are, independently for each occurrence, hydrogen or an optionally substituted aliphatic, optionally substituted cycloaliphatic, optionally substituted heterocycloaliphatic, optionally substituted aryl, or optionally substituted heteroaryl moiety or an N or S protecting group, or R₁₂ and R₁₃ taken together form a saturated or unsaturated cyclic ring containing 1 to 4 carbon atoms and 1 to 3 nitrogen or oxygen atoms; each of R₁₂ and R₁₃ being optionally substituted with one or more occurrences of hydroxyl, protected hydroxyl, alkoxy, amino, protected amino, —NH(alkyl), aminoalkyl, or halogen;
X₁ and X₂ are each independently absent, oxygen, NH, or —N(alkyl), or wherein X₂—R₁₄ together are N₃ or are a heterocycloaliphatic moiety;
p is an integer from 2 to 10, inclusive; and
R₁₄ is hydrogen or an aryl, heteroaryl, alkylaryl, or alkylheteroaryl moiety, or is —(C═O)NHR₁₅, —(C═O)OR₁₅, or —(C═O)R₁₅, wherein each occurrence of R₁₅ is independently hydrogen or an aliphatic, cycloaliphatic, heterocycloaliphatic, aryl, or heteroaryl moiety; or R₁₄ is —SO₂(R₁₆), wherein R₁₆ is an aliphatic moiety; wherein one or more of R₁₄, R₁₅, and R₁₆ is optionally substituted with one or more occurrences of hydroxyl, protected hydroxyl, alkoxy, amino, protected amino, —NH(alkyl), aminoalkyl, or halogen;
or R₈ and R₉, when taken together, form a saturated or unsaturated cyclic ring containing 1 to 4 carbon atoms and 1 to 3 nitrogen or oxygen atoms, said ring being optionally substituted with hydroxyl, protected hydroxyl, alkoxy, amino, protected amino, —NH(alkyl), aminoalkyl, or halogen;
R₁₀ is hydrogen, hydroxyl, alkoxy, hydroxyalkyl, halogen, or protected hydroxyl;
R₁₁ is hydrogen, hydroxyl, protected hydroxyl, amino, or protected amino;
R₂₀ is hydrogen, or R₂₀ and R₂ combine to form a bond;
X is absent or is O, NH, N-alkyl, CH₂, or S;
Y and Z are connected by a single or double bond, with Y being CHR₁₇, O, C═O, CR₁₇, or NR₁₇ and with Z being CHR₁₈, O, C═O, CR₁₈, or NR₁₈; wherein R₁₇ and R₁₈ are, independently for each occurrence, hydrogen or an optionally substituted aliphatic moiety, or R₁₇ and R₁₈ taken together are —O—, —CH₂— or —NR₁₉—, wherein R₁₉ is hydrogen or alkyl;
and the pharmaceutically acceptable salts and derivatives thereof, said compound being other than a naturally occurring resorcylic acid lactone, hypothemycin, (5Z)-7-oxozeaneol, Ro-09-2210, and L-783,277.

2. The method of claim 1, wherein said compound comprises an enone moiety that forms a Michael adduct with said Cys, and said compound has a cis carbon-carbon double bond at positions 5-6 conjugated to a carbonyl at position 7.

3. A method according to claim 1, wherein the compound has a structure according to formula Ia 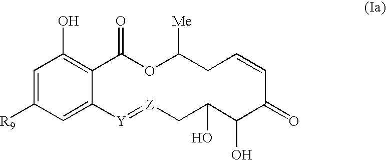
wherein
Y and Z are connected by a single or double bond, with Y being CHR₁₇, and with Z being CHR₁₈; wherein R₁₇ and R₁₈ are hydrogen, or R₁₇ and R₁₈ taken together are —O—; or
wherein the compound has a structure according to formula Ic 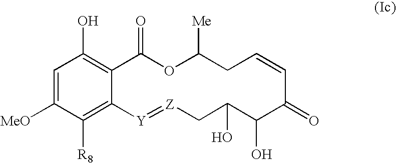
wherein
R₈ is hydrogen, halogen, hydroxyl, protected hydroxyl, alkoxy, or an aliphatic moiety optionally substituted with hydroxyl, protected hydroxyl, SR₁₂, or NR₁₂R₁₃; and
Y and Z are connected by a single or double bond, with Y being CHR₁₇, and with Z being CHR₁₈; wherein R₁₇ and R₁₈ are hydrogen, or R₁₇ and R₁₈ taken together are —O—; or
wherein the compound has a structure according to formula Id 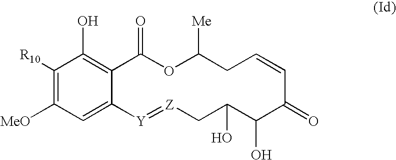
wherein
R₁₀ is hydrogen, hydroxyl, alkoxy, hydroxyalkyl, halogen, or protected hydroxyl; and
Y and Z are connected by a single or double bond, with Y being CHR₁₇, and with Z being CHR₁₈; wherein R₁₇ and R₁₈ are hydrogen, or R₁₇ and R₁₈ taken together are —O—; or
wherein the compound has the structure according to formula Ie 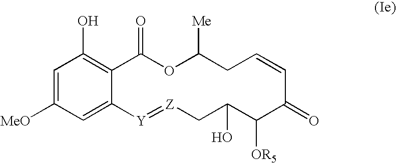
wherein
R₅ is hydrogen, C₂ to C₅ alkyl, an oxygen protecting group or a prodrug moiety; and
Y and Z are connected by a single or double bond, with Y being CHR₁₇, and with Z being CHR₁₈; wherein R₁₇ and R₁₈ are hydrogen, or R₁₇ and R₁₈ is taken together are —O—; or
wherein the compound has the structure according to formula If 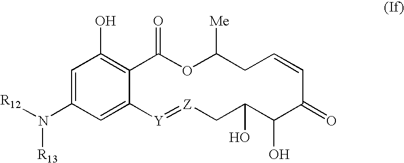
wherein R₁₂ and R₁₃ are, independently for each occurrence, hydrogen or an optionally substituted aliphatic, optionally substituted cycloaliphatic, optionally substituted heterocycloaliphatic, optionally substituted aryl, or optionally substituted heteroaryl moiety or an N or S protecting group, or R₁₂ and R₁₃ taken together form a saturated or unsaturated cyclic ring containing 1 to 4 carbon atoms and 1 to 3 nitrogen or oxygen atoms; each of R₁₂ and R₁₃ being optionally substituted with one or more occurrences of hydroxyl, protected hydroxyl, alkoxy, amino, protected amino, —NH(alkyl), aminoalkyl, or halogen;
Y and Z are connected by a single or double bond, with Y being CHR₁₇, and with Z being CHR₁₈; wherein R₁₇ and R₁₈ are hydrogen, or R₁₇ and R₁₈ taken together are —O—; or
wherein the compound has the structure according to formula Ig 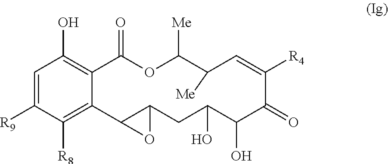
wherein
R₄ is H or F;
R₈ is H; and
R₉ is selected from the group consisting of 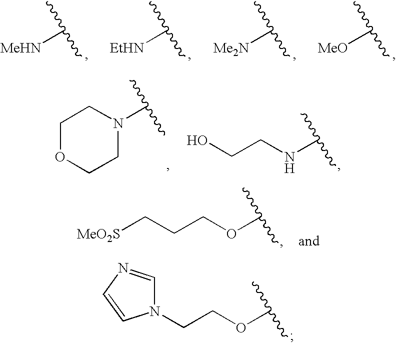
or R₈ and R₉ combine to form

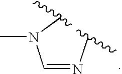

4. A method for treating a disease or disease condition by administering to a patient in need of treatment for said disease or disease condition a pharmaceutical composition that comprises a compound that specifically inhibits a protein kinase having a cysteine residue (Cys) located between and immediately adjacent to one of two conserved aspartate residues in the ATP-binding site region of said protein kinase, said method comprising contacting said kinase with a compound that forms a Michael adduct with said. Cys, wherein said pharmaceutical composition comprises a compound of structure (I) 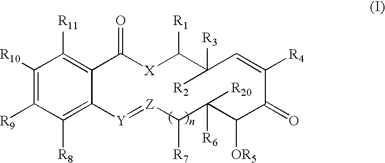
wherein
R₁ is hydrogen or an optionally substituted aliphatic, optionally substituted cycloaliphatic, optionally substituted heterocycloaliphatic, optionally substituted aryl, or optionally substituted heteroaryl moiety;
R₂ and R₃ are each independently hydrogen, halogen, hydroxyl, protected hydroxyl, or an optionally substituted aliphatic, optionally substituted cycloaliphatic, optionally substituted heterocycloaliphatic, optionally substituted aryl or optionally substituted, heteroaryl moiety; or R₁ and R₂, when taken together, form an optionally substituted, saturated or unsaturated cyclic ring of 3 to 8 carbon atoms; or R₁ and R₃, when taken together, form an optionally substituted, saturated or unsaturated cyclic ring of 3 to 8 carbon atoms;
R₄ is hydrogen or halogen;
R₅ is hydrogen, C₂ to C₅ alkyl, an oxygen protecting group or a prodrug moiety;
R₆ is hydrogen, hydroxyl, or protected hydroxyl;
n is 0, 1, or 2;
R₇ is, for each occurrence, independently hydrogen, hydroxyl, or protected hydroxyl;
R₈ is hydrogen, halogen, hydroxyl, protected hydroxyl, alkoxy, or an aliphatic moiety optionally substituted with hydroxyl, protected hydroxyl, SR₁₂, or NR₁₂R₁₃;
R₉ is hydrogen, halogen, hydroxyl, protected hydroxyl, OR₁₂, SR₁₂, NR₁₂R₁₃,
—X₁(CH₂)ₚX₂—R₁₄, or is alkyl optionally substituted with hydroxyl, protected hydroxyl, halogen, amino, protected amino, or —X₁(CH₂)ₚX₂—R₁₄;
wherein
R₁₂ and R₁₃ are, independently for each occurrence, hydrogen or an optionally substituted aliphatic, optionally substituted cycloaliphatic, optionally substituted heterocycloaliphatic, optionally substituted aryl, or optionally substituted heteroaryl moiety or an N or S protecting group, or R₁₂ and R₁₃, taken together form a saturated or unsaturated cyclic ring containing 1 to 4 carbon atoms and 1 to 3 nitrogen or oxygen atoms; each of R₁₂ and R₁₃ being optionally substituted with one or more occurrences of hydroxyl, protected hydroxyl, alkoxy, amino, protected amino, —NH(alkyl), aminoalkyl, or halogen;
X₁ and X₂ are each independently absent, oxygen, NH, or —N(alkyl), or wherein X₂—R₁₄ together are N₃ or are a heterocycloaliphatic moiety;
p is an integer from 2 to 10, inclusive; and
R₁₄ is hydrogen or an aryl, heteroaryl, alkylaryl, or alkylheteroaryl moiety, or is —(C═O)NHR₁₅, —(C═O)OR₁₅, or —(C═O)R₁₅, wherein each occurrence of R₁₅ is independently hydrogen or an aliphatic, cycloaliphatic, heterocycloaliphatic, aryl, or heteroaryl moiety; or R₁₄ is —SO₂(R₁₆), wherein R₁₆ is an aliphatic moiety; wherein one or more of R₁₄, R₁₅, and R₁₆ is optionally substituted with one or more occurrences of hydroxyl, protected hydroxyl, alkoxy, amino, protected amino, —NH(alkyl), aminoalkyl, or halogen;
or R₈ and R₉, when taken together, form a saturated or unsaturated cyclic ring containing 1 to 4 carbon atoms and 1 to 3 nitrogen or oxygen atoms, said ring being optionally substituted with hydroxyl, protected hydroxyl, alkoxy, amino, protected amino, —NH(alkyl), aminoalkyl, or halogen;
R₁₀ is hydrogen, hydroxyl, alkoxy, hydroxyalkyl, halogen, or protected hydroxyl;
R₁₁ is hydrogen, hydroxyl, protected hydroxyl, amino, or protected amino;
R₂₀ is hydrogen, or R₂₀ and R₂ combine to form a bond;
X is absent or is O, NH, N-alkyl, CH₂, or S;
Y and Z are connected by a single or double bond, with Y being CHR₁₇, O, C═O, CR₁₇, or NR₁₇ and with Z being CHR₁₈, O, CO, CR₁₈, or NR₁₈;
wherein R₁₇ and R₁₈ are, independently for each occurrence, hydrogen or an optionally substituted aliphatic moiety, or R₁₇ and R₁₈ taken together are —O—, —CH₂— or —NR₁₉—, wherein R₁₉ is hydrogen or alkyl;
and the pharmaceutically acceptable salts and derivatives thereof, said compound being other than a naturally occurring resorcylic acid lactone, hypothemycin, (5Z)-7oxozeaneol, Ro-09-2210, and L-783,277.

5. A method according to claim 4, wherein the compound has a structure according to formula Ia 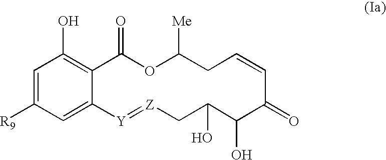
wherein
Y and Z are connected by a single or double bond, with Y being CHR₁₇, and with Z being CHR₁₈; wherein R₁₇ and R₁₈ are hydrogen, or R₁₇ and R₁₈ taken together are —O—; or
wherein the compound has the structure according to formula Ic 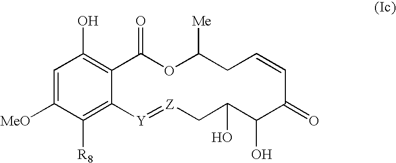
wherein
R₈ is hydrogen, halogen, hydroxyl, protected hydroxyl, alkoxy, or an aliphatic moiety optionally substituted with hydroxyl, protected hydroxyl, SR₁₂, or NR₁₂R₁₃; and
Y and Z are connected by a single or double bond, with Y being CHR₁₇, and with Z being CHR₁₈; wherein R₁₇ and R₁₈ are hydrogen, or R₁₇ and R₁₈ taken together are —O—; or
wherein the compound has the structure according to formula Id 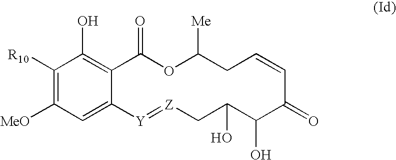
wherein
R₁₀ is hydrogen, hydroxyl, alkoxy, hydroxyalkyl, halogen, or protected hydroxyl; and
Y and Z are connected by a single or double bond, with Y being CHR₁₇, and with Z being CHR₁₈; wherein R₁₇ and R₁₈ are hydrogen, or R₁₇ and R₁₈ is taken together are —O—; or
wherein the compound has the structure according to formula Ie 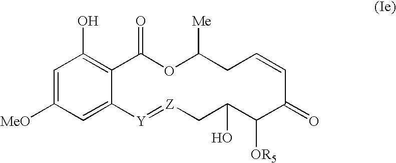
wherein
R₅ is hydrogen, C₂ to C₅ alkyl, an oxygen protecting group or a prodrug moiety; and
Y and Z are connected by a single or double bond, with Y being CHR₁₇, and with Z being CHR₁₈; wherein R₁₇ and R₁₈ are hydrogen, or R₁₇ and R₁₈ is taken together are —O—; or
wherein the compound has the structure according to formula If 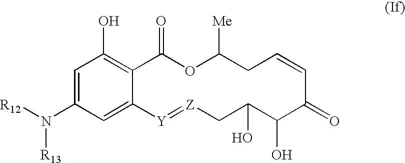
wherein
R₁₂ and R₁₃ are, independently for each occurrence, hydrogen or an optionally substituted aliphatic, optionally substituted cycloaliphatic, optionally substituted heterocycloaliphatic, optionally substituted aryl, or optionally substituted heteroaryl moiety or an N or S protecting group, or R₁₂ and R₁₃ taken together form a saturated or unsaturated cyclic ring containing 1 to 4 carbon atoms and 1 to 3 nitrogen or oxygen atoms; each of R₁₂ and R₁₃ being optionally substituted with one or more occurrences of hydroxyl, protected hydroxyl, alkoxy, amino, protected amino, —NH(alkyl), aminoalkyl, or halogen;
Y and Z are connected by a single or double bond, with Y being CHR₁₇, and with Z being CHR₁₈; wherein R₁₇ and R₁₈ are hydrogen, or R₁₇ and R₁₈ taken together are —O—; or
wherein the compound has the structure according to formula Ig 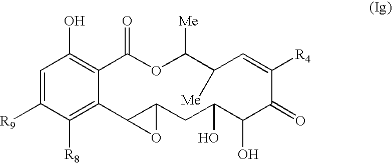
wherein
R₄ is H or F;
R₈ is H; and
R₉ is selected from the group, consisting of 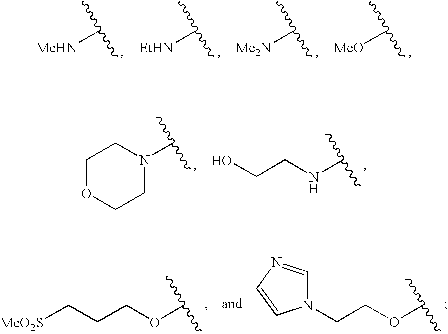
or R₈ and R₉ combine to form

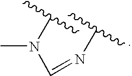

6. The method as defined in claim 1 wherein the compound has a structure according to formula II, III, IV or V

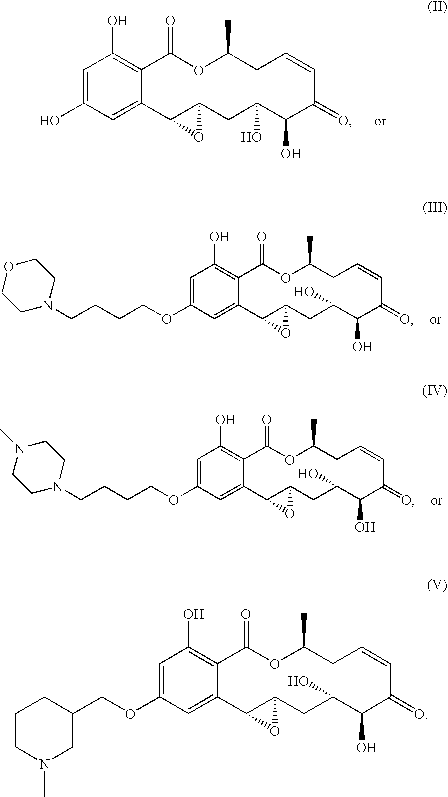

7. The method of claim 1, wherein said kinase is selected from the group consisting of AAK1, APEG1 splice variant with kinase domain (SPEG), BMP2K (BIKE), CDKL1, CDKL2, CDKL3, CDKL4, CDKL5 (STK9), ERK1 (MAPK3), ERK2 (MAPK1), FLT3, GAK, GSK3A, GSK3B, KIT (cKIT), MAP3K14 (NIK), MAP3K7 (TAK1), MAPK15 (ERK8), MAPKAPK5 (PRAK), MEK1 (MKK1, MAP2K1), MEK2 (MKK2, MAP2K2), MEK3 (MKK3, MAP2K3), MEK4 (MKK4, MAP2K4), MEK5 (MKK5, MAP2K5), MEK6 (MKK6, MAP2K6), MEK7 (MKK7, MAP2K7), MKNK1 (MNK1), MKNK2 (MNK2, GPRK7), NLK, PDGFR alpha, PDGFR beta, PRKD1 (PRKCM), PRKD2, PRKD3 (PRKCN), PRPF4B (PRP4K), RPS6KA1 (RSK1, MAPKAPK1A), RPS6KA2 (RSK3, MAPKAP1B), RPS6KA3 (RSK2, MAPKAP1C), RPS6KA6 (RSK4), STK36 (FUSED_STK), STYK1, TGFBR2, TOPIC, VEGFR¹ (FLT1), VEGFR2 (KDR), VEGFR3 (FLT4) and ZAK.

8. The method of claim 1, wherein said one or more protein kinases are ERK pathway kinases, and at least two of said ERK pathway kinases are inhibited, wherein said protein kinases are MEK1, MEK2, ERK1, and ERK2.

9. The method of claim 1, wherein said one or more protein kinases inhibited include at least two ERK MAPK cascade pathway kinases and a mitogen receptor kinase.

10. The method of claim 9, wherein the mitogen receptor kinase is selected from the group consisting of: a VEGF receptor; a PDGF receptor; cKIT (the mast cell growth factor receptor); FLT3 (the receptor for FL, the Flt3 ligand); and a constitutively activated mutant of a VEGF receptor, a PDGF receptor, cKIT, or FLT3.

11. The method of claim 4, wherein the kinase inhibitor is administered together with a microtubule stabilizing or destabilizing agent, or an Hsp90 inhibitor.

12. The method of claim 11, wherein the HSP90 inhibitor is 17-AAG or 17-DMAG.

13. The method of claim 4, wherein said kinase is selected from the group consisting of PDGFR alpha, PDGFR beta, the VEGF receptors (Flt-1, Flt-4 and Kdr), MEK1/2, and ERK1/2, and said disease is age related macular degeneration or glaucoma;
wherein said kinase is either Flt-3, c-Kit MEK, ERK, or VEGFR, and said disease is acute myelogenous leukemia;
wherein said kinase is either c-Kit, PDGFR, MEK1/2 or ERK1/2, and said disease is gastrointestinal stromal tumor;
wherein said kinase is either wild type c-Kit, a constitutively active c-Kit V816D mutant, MEK1/2 or ERK1/2, and said disease is mastocytosis; or
wherein said kinase is either MEK1/2, ERK1/2 or Tak1, and said disease is inflammatory bowel disease.

14. The method of claim 13, wherein said inflammatory bowel disease is Crohn's disease or ulcerative colitis;
said kinase activated by a mutant Raf-1 is RSK or MEK/ERK;
said inflammatory syndrome is allergic dermatitis;
said kinase is c-Kit, MEK, or ERK, and said disease is an inflammatory syndrome that is influenced by or caused by mast cells;
said kinase is either MEK1/2 or ERK1/2, and said disease is breast cancer;
said kinase is either Kdr, c-Kit, MEK1/2 or ERK1/2, and said disease is non-small cell lung cancer;
said kinase is either PDGFRA, MEK1/2 or ERK1/2 and said disease is ovarian cancer;
said kinase is either a PDGFR, MEK1/2 or ERK1/2, and said disease is pancreatic cancer;
said kinase is a kinase activated by a mutant Raf-1 protein kinase, and said disease is prostate cancer;
said kinase is either a VEGFR, a PDGFR, MEK1/2, ERK1/2, Tak1, or a kinase that activates the JNK and p38 signaling pathways, and said, disease is psoriasis;
said kinase is either a PDGFR, MEK1/2 or ERK1/2, and said disease is basal cell carcinoma;
said kinase is either MEK1/2, ERK1/2, Tak1, or a kinase that activates the JNK signaling pathway, and said disease is an inflammatory syndrome;
said kinase is a PDGFR, and said disease is pulmonary fibrosis;
said kinase is either MEK1/2 or ERK1/2, and said disease is a Ras mutant dependent cancer;
said kinase is either a VEGFR, a PDGFR, MEK1/2 or ERK1/2, and said disease is renal cell carcinoma;
said kinase is either a PDGFR, MEK1/2, ERK1/2 or Tak1, and said disease is restenosis;
said kinase is either MEK1/2, ERK1/2 or Tak1, and said disease is rheumatoid arthritis;
said kinase is a kinase in a cell signaling pathway activated by mutated B-Raf;
said kinase is either PDGFRB, PDGFRA, MEK/ERK, or KIT, and said disease is chronic myelomonocytic leukemia, glioblastoma multiforme, GIST, or metastative GIST;
said kinase is FLT3;
said disease is acute myeloid leukemia;
said kinase is either KDR, FLT4, or FLT 1;
said disease involves angiogenesis;
said disease involves lymphangiogenis;
said disease involves the induction of vascular permeability;
said disease involves inflammation; and
said disease is characterized by the proliferation of cells having mutated B-Raf.

15. The method of claim 4, wherein said disease is melanoma.

16. A compound having the structure I 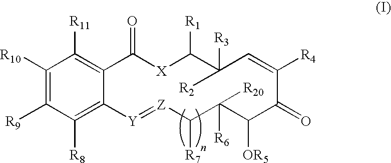
wherein
R₁ is hydrogen or an optionally substituted aliphatic, optionally substituted cycloaliphatic, optionally substituted heterocycloaliphatic, optionally substituted aryl, or optionally substituted heteroaryl moiety;
R₂ and R₃ are each independently hydrogen, halogen, hydroxyl, protected hydroxyl, or an optionally substituted aliphatic, optionally substituted cycloaliphatic, optionally substituted heterocycloaliphatic, optionally substituted aryl or optionally substituted heteroaryl moiety; or R₁ and R₂, when taken together, form an optionally substituted, saturated or unsaturated cyclic ring of 3 to 8 carbon atoms; or R₁ and R₃, when taken together, form an optionally substituted, saturated or unsaturated cyclic ring of 3 to 8 carbon atoms;
R₄ is hydrogen or halogen;
R₅ is hydrogen, C₂ to C₅ alkyl, an oxygen protecting group or a prodrug moiety;
R₆ is hydrogen, hydroxyl, or protected hydroxyl;
n is 0, 1, or 2;
R₇ is, for each occurrence, independently hydrogen, hydroxyl, or protected hydroxyl;
R₈ is hydrogen, halogen, hydroxyl, protected hydroxyl, alkoxy, or an aliphatic moiety optionally substituted with hydroxyl, protected hydroxyl, SR₁₂, or NR₁₂R₁₃;
R₉ is hydrogen, halogen, hydroxyl, protected hydroxyl, OR₁₂, SR₁₂, NR₁₂R₁₃,
—X₁(CH₂)ₚX₂—R₁₄, or is alkyl optionally substituted with hydroxyl, protected hydroxyl, halogen, amino, protected amino, or —X₁(CH₂)ₚX₂—R₁₄;
wherein
R₁₂ and R₁₃ are, independently for each occurrence, hydrogen or an optionally substituted aliphatic, optionally substituted cycloaliphatic, optionally substituted heterocycloaliphatic, optionally substituted aryl, or optionally substituted heteroaryl moiety or an N or S protecting group, or R₁₂ and R₁₃, taken together form a saturated or unsaturated cyclic ring containing 1 to 4 carbon atoms and 1 to 3 nitrogen or oxygen atoms; each of R₁₂ and R₁₃ being optionally substituted with one or more occurrences of hydroxyl, protected hydroxyl, alkoxy, amino, protected amino, —NH(alkyl), aminoalkyl, or halogen;
X₁ and X₂ are each independently absent, oxygen, NH, or —N(alkyl), or wherein X₂—R₁₄ together are N₃ or are a heterocycloaliphatic moiety;
p is an integer from 2 to 10, inclusive; and
R₁₄ is hydrogen or an aryl, heteroaryl, alkylaryl, or alkylheteroaryl moiety, or is —(C═O)NHR₁₅, —(C═O)OR₁₅, or —(C═O)R₁₅, wherein each occurrence of R₁₅ is independently hydrogen or an aliphatic, cycloaliphatic, heterocycloaliphatic, aryl, or heteroaryl moiety; or R₁₄ is —SO₂(R₁₆), wherein R₁₆ is an aliphatic moiety; wherein one or more of R₁₄, R₁₅, and R₁₆ is optionally substituted with one or more occurrences of hydroxyl, protected hydroxyl, alkoxy, amino, protected amino, —NH(alkyl), aminoalkyl, or halogen;
or R₈ and R₉, when taken together, form a saturated or unsaturated cyclic ring containing 1 to 4 carbon atoms and 1 to 3 nitrogen or oxygen atoms, said ring being optionally substituted with hydroxyl, protected hydroxyl, alkoxy, amino, protected amino, —NH(alkyl), aminoalkyl, or halogen;
R₁₀ is hydrogen, hydroxyl, alkoxy, hydroxyalkyl, halogen, or protected hydroxyl;
R₁₁ is hydrogen, hydroxyl, protected hydroxyl, amino, or protected amino;
R₂₀ is hydrogen, or R₂₀ and R₂ combine to form a bond;
X is absent or is O, NH, N-alkyl, CH₂, or S;
Y and Z are connected by a single or double bond, with Y being CHR₁₇, O, C═O, CR₁₇, or NR₁₇ and with Z being CHR₁₈, O, C═O, CR₁₈, or NR₈;
wherein R₁₇ and R₁₈ are, independently for each occurrence, hydrogen or an optionally substituted aliphatic moiety, or R₁₇ and R₁₈ taken together are —O—, —CH₂— or —NR₁₉—, wherein R₁₉ is hydrogen or alkyl;
and the pharmaceutically acceptable salts and derivatives thereof, wherein said compound is other than a naturally occurring resorcylic acid lactone, hypothemycin, (5Z)-7oxozeaneol, Ro-09-2210, and L-783,277.

17. The compound as defined in claim 16 wherein the compound has a structure according to formula Ia 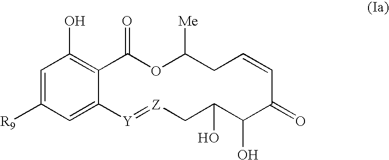
wherein
Y and Z are connected by a single or double bond, with Y being CHR₁₇, and with Z being CHR₁₈; wherein R₁₇ and R₁₈ are hydrogen, or R₁₇ and R₁₈ taken together are —O—; or
wherein the compound has a structure according to formula Ic 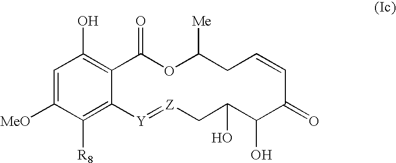
wherein
R₈ is hydrogen, halogen, hydroxyl, protected hydroxyl, alkoxy, or an aliphatic moiety optionally substituted with hydroxyl, protected hydroxyl, SR₁₂, or NR₁₂R₁₃; and
Y and Z are connected by a single or double bond, with Y being CHR₁₇, and with Z being CHR₁₈; wherein R₁₇ and R₁₈ are hydrogen, or R₁₇ and R₁₈ taken together are —O—; or
wherein the compound has a structure according to formula Id 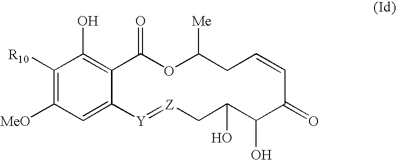
wherein
R₁₀ is hydrogen, hydroxyl, alkoxy, hydroxyalkyl, halogen, or protected hydroxyl; and
Y and Z are connected by a single or double bond, with Y being CHR₁₇, and with Z being CHR₁₈; wherein R₁₇ and R₁₈ are hydrogen, or R₁₇ and R₁₈ taken together are —O—; or
wherein the compound has the structure according to formula Ie 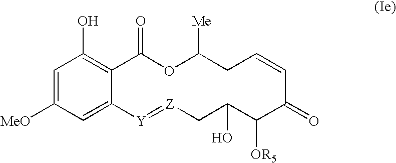
wherein
R₅ is hydrogen, C₂ to C₅ alkyl, an oxygen protecting group or a prodrug moiety; and
Y and Z are connected by a single or double bond, with Y being CHR₁₇, and with Z being CHR₁₈; wherein R₁₇ and R₁₈ are hydrogen, or R₁₇ and R₁₈ taken together are —O—; or
wherein the compound has the structure according to formula If 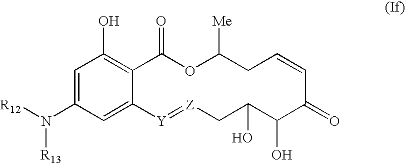
wherein R₁₂ and R₁₃ are, independently for each occurrence, hydrogen or an optionally substituted aliphatic, optionally substituted cycloaliphatic, optionally substituted heterocycloaliphatic, optionally substituted aryl, or optionally substituted heteroaryl moiety or an N or S protecting group, or R₁₂ and R₁₃ taken together form a saturated or unsaturated cyclic ring containing 1 to 4 carbon atoms and 1 to 3 nitrogen or oxygen atoms; each of R₁₂ and R₁₃ being optionally substituted with one or more occurrences of hydroxyl, protected hydroxyl, alkoxy, amino, protected amino, —NH(alkyl), aminoalkyl, or halogen;
Y and Z are connected by a single or double bond, with Y being CHR₁₇, and with Z being CHR₁₈; wherein R₁₇ and R₁₈ are hydrogen, or R₁₇ and R₁₈ taken together are —O—; or
wherein the compound has the structure according to formula Ig 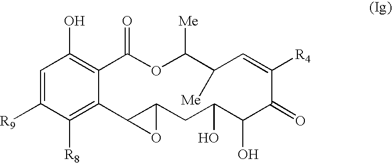
wherein
R₄ is H or F;
R₈ is H; and
R₉ is selected from the group consisting of 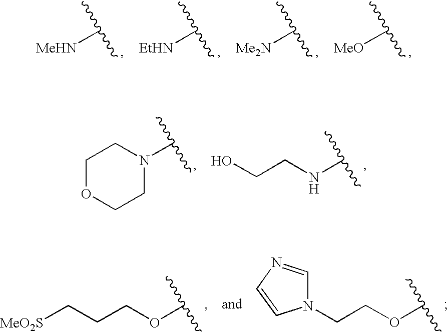
or R₈ and R₉ combine to form

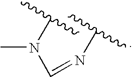

18. The compound as defined in claim 17 having a structure according to formula II, III, IV or V [chemical structure]
